(19) United States
(12) Patent Application Publication
(10) Pub. No.: US 2025/0034266 A1
(43) Pub. Date: Jan. 30, 2025

(54) CCR8 ANTIGEN BINDING UNIT AND USES THEREOF

(71) Applicants: ZAI LAB (SHANGHAI) CO., LTD., Shanghai (CN); ZAI LAB (US) LLC, Menlo Park, CA (US)

(72) Inventors: Lishan KANG, Shanghai (CN); Hongshui LIU, Shanghai (CN); Lina WANG, Shanghai (CN); Shang YIN, Shanghai (CN); Shou LI, Shanghai (CN); Bing WAN, Shanghai (CN); Wenhua SHI, Shanghai (CN); Min CHEN, Shanghai (CN); Xinchuan DAI, Shanghai (CN); David BELLOVIN, Menlo Park, CA (US); Jing ZHANG, Menlo Park, CA (US)

(21) Appl. No.: 18/715,739

(22) PCT Filed: Dec. 2, 2022
(86) PCT No.: PCT/CN2022/136276

§ 371 Date: Jun. 3, 2024

(30) Foreign Application Priority Data
Dec. 2, 2021 (WO) PCT/CN2021/134930

(51) Int. Cl. C07K 16/28; A61K 39/00; A61P 35/00

(52) U.S. Cl. CPC C07K 16/2866; A61P 35/00; C07K 16/2818; A61K 2039/505; A61K 2039/507; C07K 2317/24; C07K 2317/732; C07K 2317/76; C07K 2317/92

(57) ABSTRACT

Described here is are antibodies to CCR8 having enhanced binding, ligand blocking and antibody dependent cellular cytotoxicity. Also described is are methods of treating a cancer by targeting tumor infiltrating lymphocytes in a cancer patient using a formulation comprising a CCR8 antibody described above.

### TECHNICAL FIELD

The present disclosure is in the field of antibody-based therapeutics. More specifically, the present disclosure is directed to anti-CCR8 compositions and methods of using them to treat diseases.

### BACKGROUND

Despite the promise of immunotherapy for cancer treatment, nearly 80% of patients fail to respond to checkpoint inhibitor (CPI) therapy. Regulatory T cells (Tregs), which inhibit immune responses in the tumor microenvironment via multiple suppressive mechanisms, have been proposed to play a key role in this lack of CPI efficacy. Therefore, targeted depletion of Tregs should promote more effective antitumor immunity. CCR8 is a chemokine receptor that is selectively expressed on activated human tumor-resident Tregs, and these intratumoral CCR8+ Tregs have been shown to drive immunosuppression that can lead to poor prognosis. Thus, CCR8 can be targeted as a cancer immunotherapy by selectively depleting the intratumoral immunosuppressive Treg cells. An antibody targeting human CCR8 can cause Treg depletion in tumor and renders the tumor sensitivity to a CPI therapy such as anti-PD-1 treatment.

### SUMMARY OF THE DISCLOSURE

Disclosed herein are antigen binding units comprising a light chain complementarity-determining region (CDR) (refers to light chain variable region) and a heavy chain CDR (refers to heavy chain variable region). In some aspects, the antigen binding unit bins to CCR8 and prevents binding of CCR8 to C—C motif chemokine ligand 1 (CCL1) expressed on immune cell surfaces.

In some embodiments, the antigen binding unit when attached to a fragment crystallizable (Fc) region, and particularly a mutated Fc variant, exhibits enhanced antibody dependent cellular cytotoxicity (ADCC) thereby killing the CCR8-expressing Treg cells.

In some embodiments, the antigen binding unit comprises at least one mutation at a post translational modification (PTM) site. In some embodiments, the at least one mutation is at least one of, a N28G mutation, a N28Q mutation, a N28I mutation, a N28S mutation, a G29A mutation, a G29I mutation and a G29V mutation. In some embodiments, the mutation is a N28G mutation in light chain CDR1.

In some embodiments, the heavy chain CDR comprises HC-CDR1, HC-CDR2, and HC-CDR3, wherein the HC-CDR1, HC-CDR2, and HC-CDR3 each comprises a sequence with at least 60%, 65%, 70%, 75%, 80%, 81%, 82%, 83%, 84%, 85%, 86%, 87%, 88%, 89%, 90%, 91%, 92%, 93%, 94%, 95%, 96%, 97% 98%, or 99%, including any ranges having these values as endpoints (for example 60%-85%), identity to a sequence selected from SEQ ID NO: 13-15, 19-22, 71-73, 77, 78, 81, 83-85, 89, 91-93, 97, 98, and 112-118.

In some embodiments, the heavy chain CDR comprises HC-CDR1, HC-CDR2, and HC-CDR3, wherein the HC-CDR1, HC-CDR2, and HC-CDR3 each comprises a sequence selected from SEQ ID NO: 13-15, 19-22, 71-73, 77, 78, 81, 83-85, 89, 91-93, 97, 98, and 112-118.

In some embodiments, the heavy chain CDR comprises HC-CDR1, HC-CDR2, and HC-CDR3, wherein the HC-CDR1, HC-CDR2, and HC-CDR3 each comprises a sequence selected from SEQ ID NO: 13-15, 19-22, 89, 114, and 115.

In some embodiments, the heavy chain CDR comprises HC-CDR1, HC-CDR2, and HC-CDR3, wherein the HC-CDR1, HC-CDR2, and HC-CDR3 each comprises a sequence selected from SEQ ID NO: 83-85 and 116-118.

In some embodiments, the HC-CDR1 comprises a sequence selected from SEQ ID NO: 13, 71, 83, 115, and 117, the HC-CDR2 comprises a sequence selected from SEQ ID NO: 14, 72, 77, 84, 89, 91, 92, 97, 114, 116, and 118, and the HC-CDR3 comprises a sequence selected from SEQ ID NO: 15, 19-22, 73, 78, 81, 85, 93, 98, 112, and 113.

In some embodiments, the HC-CDR1 comprises a sequence selected from SEQ ID NO: 13 and 115, the HC-CDR2 comprises a sequence selected from SEQ ID NO: 14, 89, and 114, and the HC-CDR3 comprises a sequence selected from SEQ ID NO: 15 and 19-22.

In some embodiments, the HC-CDR1 comprises a sequence selected from SEQ ID NO: 83 and 117, the HC-CDR2 comprises a sequence selected from SEQ ID NO: 84, 116, and 118, and HC-CDR3 comprises a sequence of SEQ ID NO: 85.

In some embodiments, the light chain CDR comprises LC-CDR1, LC-CDR2, and LC-CDR3, wherein the LC-CDR1, LC-CDR2, and LC-CDR3 each comprises a sequence with at least 60%, 65%, 70%, 75%, 80%, 81%, 82%, 83%, 84%, 85%, 86%, 87%, 88%, 89%, 90%, 91%, 92%, 93%, 94%, 95%, 96%, 97% 98%, or 99%, including any ranges having these values as endpoints (for example 60%-85%), identity to a sequence selected from SEQ ID NO: 16-18, 23, 24, 25, 74-76, 79, 80, 82, 86-88, 90, 94-96, and 99-111.

In some embodiments, the light chain CDR comprises LC-CDR1, LC-CDR2, and LC-CDR3, wherein the LC-CDR1, LC-CDR2, and LC-CDR3 each comprises a sequence selected from SEQ ID NO: 16-18, 23, 24, 25, 74-76, 79, 80, 82, 86-88, 90, 94-96, and 99-111.

In some embodiments, the light chain CDR comprises LC-CDR1, LC-CDR2, and LC-CDR3, wherein the LC-CDR1, LC-CDR2, and LC-CDR3 each comprises a sequence selected from SEQ ID NO: 16-18, 23, 24, 25, 90, and 102-106.

In some embodiments, the light chain CDR comprises LC-CDR1, LC-CDR2, and LC-CDR3, wherein the LC-CDR1, LC-CDR2, and LC-CDR3 each comprises a sequence selected from SEQ ID NO: 86-88.

In some embodiments, the LC-CDR1 comprises a sequence selected from SEQ ID NO: 16, 74, 79, 86, 90, 94, 99 and 102-109, the LC-CDR2 comprises a sequence selected from SEQ ID NO: 17, 75, 80, 87, 95, 100, 110, and 111, and the LC-CDR3 comprises a sequence selected from SEQ ID NO: 18, 23-25, 76, 82, 88, 96, and 101.

In some embodiments, the LC-CDR1 comprises a sequence selected from SEQ ID NO: 16, 90, and 102-106, the LC-CDR2 comprises a sequence of SEQ ID NO: 17, and the LC-CDR3 comprises a sequence selected from SEQ ID NO: 18 and 23-25.

In embodiments, the LC-CDR1 comprises a sequence of SEQ ID NO: 86, the LC-CDR2 comprises a sequence of SEQ ID NO: 87, and the LC-CDR3 comprises a sequence of SEQ ID NO: 88.

In some embodiments, the heavy chain CDR comprises HC-CDR1, HC-CDR2, and HC-CDR3, wherein the HC-CDR1, HC-CDR2, and HC-CDR3 comprise, respectively, amino acid sequences selected from among the following groups: SEQ ID NO: 13, SEQ ID NO: 14, and SEQ ID NO: 15; SEQ ID NO:13, SEQ ID NO: 14, and SEQ ID NO: 19; SEQ ID NO:13, SEQ ID NO: 14, and SEQ ID NO: 20; SEQ ID NO:13, SEQ ID NO: 14, and SEQ ID NO: 21; SEQ ID NO:13, SEQ ID NO: 14, and SEQ ID NO: 22; SEQ ID NO:71, SEQ ID NO: 72, and SEQ ID NO: 73; SEQ ID NO:71, SEQ ID NO: 77, and SEQ ID NO: 78; SEQ ID NO: 71, SEQ ID NO: 72, and SEQ ID NO: 81; SEQ ID NO: 83, SEQ ID NO: 84, and SEQ ID NO: 85; SEQ ID NO:13, SEQ ID NO: 89, and SEQ ID NO: 15; SEQ ID NO:13, SEQ ID NO: 91, and SEQ ID NO: 15; SEQ ID NO: 71, SEQ ID NO: 92, and SEQ ID NO: 93; SEQ ID NO:13, SEQ ID NO: 97, and SEQ ID NO: 98; SEQ ID NO:13, SEQ ID NO: 97, and SEQ ID NO: 112; SEQ ID NO:13, SEQ ID NO: 97, and SEQ ID NO: 113; SEQ ID NO:13, SEQ ID NO: 114, and SEQ ID NO: 15; SEQ ID NO:115, SEQ ID NO: 114, and SEQ ID NO: 15; SEQ ID NO: 83, SEQ ID NO: 116, and SEQ ID NO: 85; SEQ ID NO:117, SEQ ID NO: 116, and SEQ ID NO: 85; and SEQ ID NO: 83, SEQ ID NO: 118, and SEQ ID NO: 85.

In some embodiments, the light chain CDR comprises LC-CDR1, LC-CDR2, and LC-CDR3, wherein the LC-CDR1, LC-CDR2, and LC-CDR3 comprise, respectively, amino acid sequences selected from among the following groups: SEQ ID NO: 16, SEQ ID NO: 17, and SEQ ID NO: 18; SEQ ID NO: 16, SEQ ID NO: 17, and SEQ ID NO: 23; SEQ ID NO: 16, SEQ ID NO: 17, and SEQ ID NO: 24; SEQ ID NO: 16, SEQ ID NO: 17, and SEQ ID NO: 25; SEQ ID NO: 74, SEQ ID NO: 75, and SEQ ID NO: 76; SEQ ID NO: 79, SEQ ID NO: 80, and SEQ ID NO: 76; SEQ ID NO: 79, SEQ ID NO: 75, and SEQ ID NO: 82; SEQ ID NO: 86, SEQ ID NO: 87, and SEQ ID NO: 88; SEQ ID NO: 90, SEQ ID NO: 17, and SEQ ID NO: 18; SEQ ID NO: 94, SEQ ID NO: 95, and SEQ ID NO: 96; SEQ ID NO: 79, SEQ ID NO: 75, and SEQ ID NO: 76; SEQ ID NO: 99, SEQ ID NO: 100, and SEQ ID NO: 101; SEQ ID NO: 101, SEQ ID NO: 17, and SEQ ID NO: 18; SEQ ID NO: 103, SEQ ID NO: 17, and SEQ ID NO: 18; SEQ ID NO: 104, SEQ ID NO: 17, and SEQ ID NO: 18; SEQ ID NO: 105, SEQ ID NO: 17, and SEQ ID NO: 18; SEQ ID NO: 106, SEQ ID NO: 17, and SEQ ID NO: 18; SEQ ID NO: 107, SEQ ID NO: 100, and SEQ ID NO: 101; SEQ ID NO: 108, SEQ ID NO: 100, and SEQ ID NO: 101; SEQ ID NO: 109, SEQ ID NO: 100, and SEQ ID NO: 101; and SEQ ID NO: 99, SEQ ID NO: 111, and SEQ ID NO: 101.

In some embodiments, the heavy chain CDR comprises HC-CDR1, HC-CDR2, and HC-CDR3, the light chain CDR comprises LC-CDR1, LC-CDR2, and LC-CDR3, wherein the HC-CDR1, HC-CDR2, HC-CDR3, LC-CDR1, LC-CDR2, and LC-CDR3 comprise, respectively, amino acid sequences selected from among the following groups: SEQ ID NO: 13, 14, 15, 16, 17, and 18; SEQ ID NO: 13, 14, 19, 16, 17, and 18, SEQ ID NO: 13, 14, 20, 16, 17, and 18; SEQ ID NO: 13, 14, 21, 16, 17, and 18; SEQ ID NO: 13, 14, 22, 16, 17, and 18; SEQ ID NO: 13, 14, 15, 16, 17, and 23; SEQ ID NO: 13, 14, 15, 16, 17, and 24; SEQ ID NO: 13, 14, 15, 16, 17, and 25; SEQ ID NO: 13, 14, 19, 16, 17, and 23; SEQ ID NO: 13, 14, 20, 16, 17, and 23; SEQ ID NO: 13, 14, 21, 16, 17, and 23; SEQ ID NO: 13, 14, 22, 16, 17, and 23; SEQ ID NO: 13, 89, 15, 90, 17, and 18; SEQ ID NO: 13, 89, 15, 102, 17, and 18; SEQ ID NO: 13, 89, 15, 103, 17, and 18; SEQ ID NO: 13, 89, 15, 104, 17, and 18; SEQ ID NO: 13, 89, 15, 16, 17, and 18; SEQ ID NO: 13, 89, 15, 105, 17, and 18; SEQ ID NO: 13, 89, 15, 106, 17, and 18; SEQ ID NO: 13, 114, 15, 16, 17, and 18; and SEQ ID NO: 115, 114, 15, 16, 17, and 18.

In some embodiments, the heavy chain CDR comprises HC-CDR1, HC-CDR2, and HC-CDR3, the light chain CDR comprises LC-CDR1, LC-CDR2, and LC-CDR3, wherein the HC-CDR1, HC-CDR2, HC-CDR3, LC-CDR1, LC-CDR2, and LC-CDR3 comprise, respectively, amino acid sequences selected from among the following groups: SEQ ID NO: 83, 84, 85, 86, 87, and 88; SEQ ID NO: 83, 116, 85, 86, 87, and 88; SEQ ID NO: 117, 116, 85, 86, 87, and 88; and SEQ ID NO: 83, 118, 85, 86, 87, and 88.

In some embodiments, the antigen binding unit comprises a heavy chain CDR comprising a sequence with at least 60%, 65%, 70%, 75% 80%, 81%, 82%, 83%, 84%, 85%, 86%, 87%, 88%, 89%, 90%, 91%, 92%, 93%, 94%, 95%, 96%, 97% 98%, or 99%, including any ranges having these values as endpoints (for example 60%-85%), identity to a sequence selected from SEQ ID NO: 1, 3, 4, 5, 6, 7, 11, 12, 26, 28, 30, 32, 34, 36, 37, 39, 41, 54-59, 61, and 66-70.

In some embodiments, the antigen binding unit comprises a heavy chain CDR comprising a sequence selected from SEQ ID NO: 1, 3, 4, 5, 6, 7, 11, 12, 26, 28, 30, 32, 34, 36, 37, 39, 41, 54-59, 61, and 66-70.

In some embodiments, the antigen binding unit comprises a light chain CDR comprising a sequence with at least 60%, 65%, 70%, 75%, 80%, 81%, 82%, 83%, 84%, 85%, 86%, 87%, 88%, 89%, 90%, 91%, 92%, 93%, 94%, 95%, 96%, 97% 98%, or 99%, including any ranges having these values as endpoints (for example 60%-85%), identity to a sequence selected from SEQ ID NO: 2, 8, 9, 10, 27, 29, 31, 33, 35, 38, 40, 42-53, 60, and 62-65.

In some embodiments, the antigen binding unit comprises a light chain CDR comprising a sequence selected from SEQ ID NO: 2, 8, 9, 10, 27, 29, 31, 33, 35, 38, 40, 42-53, 60, and 62-65.

In some embodiments, the antigen binding unit comprises a heavy chain CDR and a light chain CDR, wherein the heavy chain CDR and the light chain CDR comprise, respectively, amino acid sequences selected from the following groups: SEQ ID NO: 1 and 2; SEQ ID NO: 3 and 2; SEQ ID NO: 4 and 2; SEQ ID NO: 5 and 2; SEQ ID NO: 6 and 2; SEQ ID NO: 7 and 2; SEQ ID NO: 1 and 8; SEQ ID NO: 1 and 9; SEQ ID NO: 1 and 10; SEQ ID NO: 3 and 8; SEQ ID NO: 4 and 8; SEQ ID NO: 5 and 8; SEQ ID NO: 6 and 8; SEQ ID NO: 11 and 8; SEQ ID NO: 12 and 8; SEQ ID NO: 34 and 35; SEQ ID NO: 34 and 43; SEQ ID NO: 34 and 44; SEQ ID NO: 34 and 45; SEQ ID NO: 34 and 46; SEQ ID NO: 34 and 47; SEQ ID NO: 34 and 48; SEQ ID NO: 56 and 2; SEQ ID NO: 57 and 2; SEQ ID NO: 58 and 2; SEQ ID NO: 59 and 2; SEQ ID NO: 56 and 60; SEQ ID NO: 57 and 60; SEQ ID NO: 58 and 60; and SEQ ID NO: 58 and 60. In some embodiments, the heavy chain CDR and the light chain CDR comprise, respectively, amino acid sequences selected from the following groups: SEQ ID NO: 1 and 2; SEQ ID NO: 3 and 2; SEQ ID NO: 4 and 2; SEQ ID NO: 5 and 2; SEQ ID NO: 6 and 2; SEQ ID NO: 7 and 2; SEQ ID NO: 1 and 8; and SEQ ID NO: 1 and 9.

In some embodiments, the antigen binding unit comprises a heavy chain CDR and a light chain CDR, wherein the heavy chain CDR and the light chain CDR comprise, respectively, amino acid sequences selected from the following groups: SEQ ID NO: 32 and 33; SEQ ID NO: 61 and 62; SEQ ID NO: 61 and 63; SEQ ID NO: 61 and 64; SEQ ID NO: 61 and 65; SEQ ID NO: 66 and 62; SEQ ID NO: 67 and 62; SEQ ID NO: 68 and 62; SEQ ID NO: 69 and 62; and SEQ ID NO: 70 and 62.

In some embodiments, the antigen binding unit comprises an IgG1 framework, with or without mutation at the Fc region.

In some embodiments, the antigen binding unit is a monoclonal antibody, a humanized antibody, or a chimeric antibody. In some embodiments, the antigen binding unit of is a scFv, a Fab′, a single chain Fab (scFab′), a Fd or a F(ab′)₂, a sFC, a Fv, or a ccFv. In some embodiments, the antigen binding unit competes for binding to an epitope recognized by an antigen binding unit.

In other embodiments, pharmaceutical compositions comprising any one of the antigen binding units disclosed herein and a pharmaceutically acceptable excipient are provided.

In other embodiments, isolated nucleic acids encoding any one of the antigen binding units disclosed herein are provided.

In other embodiments, vectors comprising a nucleic acid sequence encoding any one of the antigen binding units disclosed herein are provided.

In other embodiments, host cells expressing any one of the antigen binding units disclosed herein are provided.

In other embodiments, host cells comprising a nucleic acid encoding any one of the antigen binding units disclosed herein are provided.

In other embodiments, methods of producing any one of the antigen binding units disclosed herein are provided. The methods comprise culturing any of the host cells disclosed herein under conditions suitable for expressing the antigen binding unit; and isolating said antigen binding unit expressed by the host cell.

In other embodiments, methods of eradicating a population of immune cells are provided. The methods comprise contacting the immune cells with any of the antigen binding units disclosed herein. In some aspects, the immune cell is a regulatory T cell (Treg). In other embodiments, the immune cell is a Treg resident in a tumor.

In other embodiments, methods of treating a cancer in a subject in need thereof are provided. The methods comprise administering to a subject in need thereof an effective amount of the antigen binding unit described herein. In embodiments, the methods comprise repeating the administration step for a period of time, such as until the subject is free of the cancer. In some embodiments, the cancer is a hematological cancer or a solid tumor. In some embodiments, treating the cancer comprises reducing tumor volume.

In other embodiments, a method for treating a cancer in a subject in need thereof is provided. The method comprises administering to a subject in need thereof, an effective amount of the pharmaceutical composition comprising a pharmaceutically acceptable excipient and any antigen binding unit disclosed herein. In embodiments, the methods comprise repeating the administration step for a period of time, such as until the subject is free of the cancer. In some embodiments, the cancer is a hematological cancer or a solid tumor. In some embodiments, treating the cancer comprises reducing tumor volume.

### BRIEF DESCRIPTION OF THE DRAWINGS

FIG. 1 shows multiple alignment of amino acid sequences for human, cynomolgus and mouse and rat CCR8.

FIGS. 2A-2C show that antibodies with post-translational modification (PTM) sites removed bind to HEK293-human CCR8, HEK293 cells, and HEK293- Cynomolgus CCR8.

FIGS. 3A-3B shows that antibodies with post-translational modification (PTM) sites removed block human CCL1 binding to CHO-K1-human CCR8 cells.

FIGS. 4A-4B show binding of indicated humanized antibodies to ExpiCHO-S-CCR1 and ExpiCHO-S-CCR4 cells.

FIGS. 5A-5B shows antibody dependent cellular cytotoxicity (ADCC) activities of humanized antibodies on CHO-K1-human CCR8 cell line.

FIG. 6 shows anti-CCR8 inhibited CCL1 induced β-arrestin recruitment.

FIG. 7 shows ability of humanized anti-CCR8 antibody to block CCL1 induced cell migration.

FIGS. 8A-8B shows FACS binding of periplasmic extract (PPE) to CHO-K1-human CCR8 cells.

FIGS. 9A-9B shows ability of affinity matured antibodies to inhibit CCL1 induced (3-arrestin recruitment.

FIG. 10 shows that matured anti-CCR8 antibody block cell migration induced by CCL1.

FIGS. 11A-11B show binding of Fc variants of humanized 149 and humanized 348 antibodies to HuT78 cells.

FIGS. 12A-12B show ADCC activities of V variant CD16a/NFAT-Jurkat Cells on CHO-K1-human CCR8 cells.

FIGS. 13A-13B show ADCC activities of F variant CD16a/NFAT-Jurkat cells on CHO-K1-human CCR8 cells.

FIGS. 14A-14B show ADCC activities of V variant CD16a/NFAT-Jurkat Cells on HuT78 cells.

FIGS. 15A-15B show ADCC activities of F variant CD16a/NFAT-Jurkat cells on HuT78 cells.

FIGS. 16A-16B show ADCC activities of human PBMCs Cells on CHO-K1-human CCR8 cells.

FIGS. 17A-17B show ability of affinity matured antibodies to inhibit CCL1 induced (3-arrestin recruitment.

FIG. 18 shows that CCR8 is not expressed by major immune cell population in the peripheral blood. Kidney cancer patient PBMC (patient number 200003119) were analyzed by flow cytometry.

FIG. 19 shows that CCR8 was only expressed by Tregs from hDTCs. Kidney hDTCs from patient number 200003119 were analyzed by flow cytometry.

FIGS. 20A-20N show that CCR8 was expressed by Tregs from hDTCs in different cancer types. Two CCR8 subsets (subset 1: CCR8 lo and subset 2: CCR8 hi) were observed.

FIG. 21 shows treatment with Hu149-11G1m significantly reduced tumor volume.

FIG. 22 shows tumor volume in different treatment groups (Control, Hu149-11G1m, Anti-PD-1, and Combo) versus days post tumor implant in a study using hCCR8 knockin transgenic mice inoculated with M38 tumors

FIG. 23 shows Kaplan-Meier survival plots having animal survival percentage (%) in different treatment groups (Control, Hu149-11G1m, Anti-PD-1, and Combo) versus days post tumor inoculation in a study using hCCR8 knockin transgenic mice inoculated with M38 tumors.

FIG. 24 shows tumor volume in different treatment groups (Control, Hu149-11G1m, Anti-PD-1, and Combo) versus days post tumor implant in a study using hCCR8 knockin transgenic mice inoculated with E0771 tumors.

FIG. 25 shows Kaplan-Meier survival plots having animal survival percentage (%) in different treatment groups (Control, Hu149-11G1m, Anti-PD-1, and Combo) versus days post tumor inoculation in a study using hCCR8 knockin transgenic mice inoculated with E0771 tumors.

FIG. 26 shows flow cytometric analysis of immune cell populations in E0771 tumors on Day 7 after treatment initiation.

### DETAILED DESCRIPTION OF THE DISCLOSURE

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

It is intended that the following claims define the scope of the invention and that methods and structures within the scope of these claims and their equivalents be covered thereby.

### I. Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is understood that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges and are also encompassed, subject to any specifically excluded limit in the stated range. Where the stated range includes one or both of the limits, ranges excluding either or both of those included limits are also included.

All publications and patent applications mentioned in this specification are herein incorporated by reference to the same extent as if each individual publication or patent application was specifically and individually indicated to be incorporated by reference.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the topical compositions and all measurements made are at about 25° C., unless otherwise specified.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government or listed in the U.S. Pharmacopeia or other generally recognized pharmacopeia for use in mammals (e.g., animals), and more particularly, in humans.

The term “treating” is used herein, for instance, in reference to methods of treating cancer, and generally includes the administration of a compound or composition which reduces the frequency of, or delays the onset of, symptoms of a medical condition (e.g., cancer) in a subject relative to a subject not receiving the compound or composition. This can include reversing, reducing, or arresting the symptoms, clinical signs, and underlying pathology of a condition in a manner to improve or stabilize a subject's condition (e.g., regression of tumor growth).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs and peptidomimetics. A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence.

The term “antigen binding unit” as used herein refers to an immunoglobulin molecule and immunologically active portions of immunoglobulin molecule, i.e., a molecule that contains an antigen-binding site which specifically binds (“immunoreacts with”) an antigen. Also encompassed within the term “antigen binding unit” are immunoglobulin molecules of a variety of species origins including invertebrates and vertebrates. Structurally, the simplest naturally occurring antibody (e.g., IgG) comprises four polypeptide chains, two heavy (H) chains and two light (L) chains inter-connected by disulfide bonds. The immunoglobulins represent a large family of molecules that include several types of molecules, such as IgD, IgG, IgA, IgM and IgE. The term “immunoglobulin molecule” includes, for example, hybrid antibodies, or altered antibodies, and fragments thereof. It has been shown that the antigen binding function of an antibody can be performed by fragments of a naturally occurring antibody. These fragments are collectively termed “antigen-binding units”. Also encompassed within the term “antigen binding unit” is any polypeptide chain-containing molecular structure that has a specific shape which fits to and recognizes an epitope, where one or more non-covalent binding interactions stabilize the complex between the molecular structure and the epitope.

An antigen binding unit “specifically binds to” or “immunoreactive with” an antigen if it binds with greater affinity or avidity than it binds to one or more other reference antigens including polypeptides or other substances.

“Antigen” as used herein means a substance that is recognized and bound specifically by an antigen binding unit. Antigens can include peptides, proteins, glycoproteins, polysaccharides, and lipids; portions thereof and combinations thereof. Non-limiting exemplary antigen included CCR8 from human, murine, and other homologues thereof.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

A “chimeric” protein contains at least one fusion polypeptide comprising regions in a different position in the sequence than what occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

A gene “database” denotes a set of stored data which represent a collection of sequences including nucleotide and peptide sequences, which in turn represent a collection of biological reference materials.

“Domain” refers to a portion of a protein that is physically or functionally distinguished from other portions of the protein or peptide. Physically defined domains include those amino acid sequences that are exceptionally hydrophobic or hydrophilic, such as those sequences that are membrane-associated or cytoplasm-associated. Domains may also be defined by internal homologies that arise, for example, from gene duplication. Functionally defined domains have a distinct biological function(s). The ligand-binding domain of a receptor, for example, is that domain that binds ligand. An antigen-binding domain refers to the part of an antigen-binding unit or an antibody that binds to the antigen. Functionally defined domains need not be encoded by contiguous amino acid sequences. Functionally defined domains may contain one or more physically defined domain. Receptors, for example, are generally divided into the extracellular ligand-binding domain, a transmembrane domain, and an intracellular effector domain.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectively referred to as gene product. If the polynucleotide is derived from genomic DNA, expression may include splicing of the mRNA in a eukaryotic cell.

A “cell line” or “cell culture” denotes bacterial, plant, insect or higher eukaryotic cells grown or maintained in vitro. The descendants of a cell may not be completely identical (either morphologically, genotypically, or phenotypically) to the parent cell.

A “fusion gene” is a gene composed of at least two heterologous polynucleotides that are linked together.

The terms “gene” or “gene fragment” are used interchangeably herein. They refer to a polynucleotide containing at least one open reading frame that is capable of encoding a particular protein after being transcribed and translated. A gene or gene fragment may be genomic, cDNA, or synthesized, as long as the polynucleotide contains at least one open reading frame, which may cover the entire coding region or a segment thereof.

“Heterologous” means derived from a genotypically distinct entity from the rest of the entity to which it is being compared. For example, a promoter removed from its native coding sequence and operatively linked to a coding sequence other than the native sequence is a heterologous promoter. The term “heterologous” as applied to a polynucleotide, a polypeptide, means that the polynucleotide or polypeptide is derived from a genotypically distinct entity from that of the rest of the entity to which it is being compared. For instance, a heterologous polynucleotide or antigen may be derived from a different species origin, different cell type, and the same type of cell of distinct individuals.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector. A “host cell” can refer to a prokaryotic cell, a eukaryotic cell, or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

As used herein, the term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. Enrichment can be measured on an absolute basis, such as weight per volume of solution, or it can be measured in relation to a second, potentially interfering substance present in the source mixture. Increasing enrichments are preferred, in some embodiments. Thus, for example, a 2-fold enrichment is preferred, 10-fold enrichment is more preferred, 100-fold enrichment is more preferred, 1000-fold enrichment is even more preferred. A substance can also be provided in an isolated state by a process of artificial assembly, such as by chemical synthesis or recombinant expression.

“Linked” and “fused” or “fusion” are used interchangeably herein. These terms refer to the joining together of two more chemical elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature). Although the reading frame is thus made continuous throughout the fused segments, the segments may be physically or spatially separated by, for example, in-frame linker sequence (e.g., “flexon”).

“Operably linked” or “operatively linked” refers to a juxtaposition wherein the components so described are in a relationship permitting them to function in their intended manner. For instance, a promoter sequence is operably linked to a coding sequence if the promoter sequence promotes transcription of the coding sequence. For example, a peptide sequence (e.g. Fc) is linked to another peptide sequence (e.g. antigen binding unit) in a manner that enables a functional structure (e.g. an antibody).

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, primers, oligonucleotides, or synthesized DNA. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Recombinant” as applied to a polynucleotide means that the polynucleotide is the product of various combinations of cloning, restriction and/or ligation steps, and other procedures that result in a construct that is distinct from a polynucleotide found in nature.

In the context of polypeptides, a “sequence” is an order of amino acids in a polypeptide in an amino to carboxyl terminus direction in which residues that neighbor each other in the sequence are contiguous in the primary structure of the polypeptide. A sequence can also be a linear sequence of part of a polypeptide which is known to comprise additional residues in one or both directions.

A “vector” is a nucleic acid molecule, preferably self-replicating, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, e.g. mouse, rat, rabbit, pig, primate, including humans and other apes, particularly a human, and includes: (a) preventing the disease or symptom from occurring in a subject which may be predisposed to the disease or symptom but has not yet been diagnosed as having it; (b) inhibiting the disease symptom; (c) arresting development of the disease; (d) relieving the disease symptom; (e) causing regression of the disease or symptom; or any combination thereof.

The terms “recipient”, “individual”, “subject”, “host”, and “patient”, can be used interchangeably herein and refer to any mammalian subject for whom diagnosis, treatment, or therapy is desired, particularly humans.

The terms “cancer”, “neoplasm”, “tumor”, and “carcinoma” are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.).

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### II. Antigen Binding Units, Compositions, and Methods

In one embodiment, the present disclosure provides an antigen binding unit comprising a light chain complementarity determining region (CDR) and a heavy chain CDR.

In another embodiment, the present disclosure provides an antigen binding unit comprising a light chain CDR and a heavy chain CDR, wherein the antigen binding unit specifically binds to CCR8 and/or blocks CCL1 binding to CCR8.

In yet another embodiment, the present disclosure provides an antigen binding unit comprising a light chain CDR and a heavy chain CDR, and a fragment crystallizable (Fc) region operably linked to the antigen binding unit, wherein the antigen binding unit specifically binds to CCR8 and/or blocks CCL1 binding to CCR8.

In yet another embodiment, the present disclosure provides an antigen binding unit comprising a light chain CDR and a heavy chain CDR, and a fragment crystallizable (Fc) region comprising a mutation in at least one amino acid position, operably linked to the antigen binding unit, wherein the antigen binding unit specifically binds to CCR8 and/or blocks CCL1 binding to CCR8 and wherein the mutation in the Fc region enhances antibody dependent cellular cytotoxicity (ADCC).

In some embodiments, the fragment crystallizable (Fc) region comprises at least a portion of a human immunoglobulin constant region (Fc) with or without any mutations. In some embodiments, the mutation comprises a S239D mutation, a I332E mutation, a L235V mutation, a F243L mutation, a R292P mutation, a Y300L mutation, a P396L mutation, or a combination thereof

In some embodiments, the fragment crystallizable (Fc) region comprises at least a portion of human IgG1 (Fc) without mutation (SEQ ID NO: 119), or with mutations including S239D/I332E (SEQ ID NO: 120) or L235V/F243L/R292P/Y300L/P396L (SEQ ID NO: 121), wherein the mutation in the Fc region enhances antibody dependent cellular cytotoxicity (ADCC).

In some embodiments, the fragment crystallizable (Fc) region comprises an amino acid sequence selected from SEQ ID NO: 119-121:

SEQ ID NO: 119
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 120
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPEEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 121
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELVGGPSVFLLPPKPKDTLMISRTPEVTCVVVDVS
HEDPEVKFNWYVDGVEVHNAKTKPPEEQYNSTLRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPLVLDSDGSFFLYSKLTVDKSRW
QQGNVFSCSVMHEALHNHYTQKSLSLSPGK.

In any of the embodiments disclosed herein, an antigen binding unit comprises a light chain CDR (labeled as vL or LC in Tables 1-3). A light chain CDR can be a complementarity determining region of a light chain of an antigen binding unit. A light chain CDR can comprise a continuous sequence of amino acid residues, or two or more contiguous sequences of amino acid residues separated by, and optionally flanked by, non-complementarity determining regions, such as framework regions. In some examples, a light chain CDR comprises two or more light chain CDRs, which can be referred to as light chain CDR-1, CDR-2, and so on. In advantageous examples, a light chain CDR comprises three light chain CDRs, which can be referred to as light chain CDR-1, light chain CDR-2, and light chain CDR-3 respectively. In some examples, a group of CDRs present on a common light chain can collectively be referred to as light chain CDRs.

In other embodiments, an antigen binding unit comprises a heavy chain CDR (labeled as vH or HC in Tables 1-3). A heavy chain CDR can be a complementarity determining region of a heavy chain of an antigen binding unit. A heavy chain CDR can comprise a continuous sequence of amino acid residues, or two or more contiguous sequences of amino acid residues separated by, and optionally flanked by, non-complementarity determining regions, such as framework regions.

In some examples, a heavy chain CDR comprises two or more heavy chain CDRs, which can be referred to as heavy chain CDR-1, CDR-2, and so on. In advantageous examples, a heavy chain CDR comprises three heavy chain CDRs, which can be referred to as heavy chain CDR-1, heavy chain CDR-2, and heavy chain CDR-3 respectively. In some examples, a group of CDRs present on a common heavy chain can collectively be referred to as heavy chain CDRs.

In other embodiments, a subject antigen binding unit specifically binds to CCR8. CCR8, as used herein can also refer to orthologues, homologues, codon-optimized forms, truncated forms, fragmented forms, mutated forms, or any other known derivative form of a CCR8 sequence. For example, CCR8 can be human CCR8, a murine CCR8 or a cynomolgus CCR8.

Binding specificity can be determined by complementarity determining regions, or CDRs, such as light chain CDRs or heavy chain CDRs. In many cases, binding specificity is determined by light chain CDRs and heavy chain CDRs. A given combination of heavy chain CDRs and light chain CDRs provides a given binding pocket that confers greater affinity and/or specificity towards CCR8 as compared to other reference antigens.

Binding of an antigen binding unit to CCR8 can be characterized or expressed by any method known in the art. For example, binding can be characterized by binding affinity, which can be the strength of the interaction between the antigen binding unit and the antigen. Binding affinity can be determined by any method known in the art, such as in vitro binding assays. For example, binding affinity of antigen binding units disclosed herein can be determined when assayed in an in vitro binding assay utilizing cells expressing CCR8. Binding affinity of subject antigen binding unit can be expressed in term of Kd, which is the equilibrium dissociation constant between an antibody and its respective antigen. In some cases, antigen binding units as disclosed herein specifically bind to CCR8 with a Kd within a range of about 10 μM to about 1 fM. For example, an antigen binding unit can specifically bind to CCR8 with a Kd of less than about 10 pM, 1 pM, 0.1 pM, 10 nM, 1 nM, 0.1 nM, 10 pM, 1 pM, 0.1 pM, 10 fM, 1 fM, 0.1 fM, or less than 0.1 fM.

In some embodiments, an antigen binding unit reduces or even prevents binding of CCR8 to CCL1, and thereby among other effects, inhibits recruitment of regulatory T cells and/or recruitment of signaling factors such as for example, β-arrestin.

In embodiments, an antigen binding unit comprises a light chain CDR and a heavy chain CDR. Subject antigen binding units can comprise any of the sequences listed in Table 1. Additionally, or alternatively, a subject antigen binding unit can comprise a sequence with at least 60%, 65%, 70%, 75%, 80%, 81%, 82%, 83%, 84%, 85%, 86%, 87%, 88%, 89%, 90%, 91%, 92%, 93%, 94%, 95%, 96%, 97% 98%, or 99%, including any ranges having these values as endpoints (for example 60%-85%), identity to any of the sequences listed in Tables 1-3.

In some embodiments, an antigen binding unit comprises a light chain CDR and a heavy chain CDR, wherein the light chain CDR and the heavy chain CDR comprise, respectively, the LC-CDR and the HC-CDR selected from any combination of light CDR (vL or LC) or heavy chain CDR (vH or HC) sequences listed in Tables 1 and 2.

TABLE 1
Amino acid sequence of variable region
and CDR definition by Kabat naming
system

| SEQ | | |
|---|---|---|
| ID | | Amino acid sequence |
| NO. | Clone ID | of variable region |
| 1 | Hu149-9 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGRDYG |
| | | SSYAMDYWGQGTLVT |
| | | VSS |
| 2 | Hu149-9 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLEYPLTFGG |
| | | GTKVELK |
| 3 | Hu149-11 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGREYG |
| | | SSYAMDYWGQGTLVT |
| | | VSS |
| 2 | Hu149-11 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLEYPLTFGG |
| | | GTKVELK |
| 4 | Hu149-12 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGKEYG |
| | | SSYAMDYWGQGTLVT |
| | | VSS |
| 2 | Hu149-12 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLEYPLTFGG |
| | | GTKVELK |
| 5 | Hu149-13 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGREYG |
| | | RSYAMDYWGQGTLVT |
| | | VSS |
| 2 | Hu149-13 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLEYPLTFGG |
| | | GTKVELK |
| 6 | Hu149-14 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGREYG |
| | | SAYAMDYWGQGTLVT |
| | | VSS |
| 2 | Hu149-14 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLEYPLTFGG |
| | | GTKVELK |
| 7 | Hu149-15 vH | EVQLVESGGGLVQPG |
| | | GSLKLSCAASGFTFN |
| | | TYAMNWVRQAPGKGL |
| | | EWVGRIRSKSNFYAT |
| | | AYAASVKGRFTISRD |
| | | DSKNTAYLQMNSLKT |
| | | EDTAVYYCTRGRDYG |
| | | SSYAMDYWGQGTLVT |
| | | VSS |
| 2 | Hu149-15 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLEYPLTFGG |
| | | GTKVELK |
| 1 | Hu149-16 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGRDYG |
| | | SSYAMDYWGQGTLVT |
| | | VSS |
| 8 | Hu149-16 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLKYPLTFGG |
| | | GTKVELK |
| 1 | Hu149-17 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGRDYG |
| | | SSYAMDYWGQGTLVT |
| | | VSS |
| 9 | Hu149-17 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLSYPLTFGG |
| | | GTKVELK |
| 1 | Hu149-18 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGRDYG |
| | | SSYAMDYWGQGTLVT |
| | | VSS |
| 10 | Hu149-18 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLRYPLTFGG |
| | | GTKVELK |
| 3 | Hu149-19 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGREYG |
| | | SSYAMDYWGQGTLVT |
| | | VSS |
| 8 | Hu149-19 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLKYPLTFGG |
| | | GTKVELK |
| 4 | Hu149-20 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGKEYG |
| | | SSYAMDYWGQGTLVT |
| | | VSS |
| 8 | Hu149-20 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLKYPLTFGG |
| | | GTKVELK |
| 5 | Hu149-21 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGREYG |
| | | RSYAMDYWGQGTLVT |
| | | VSS |
| 8 | Hu149-21 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLKYPLTFGG |
| | | GTKVELK |
| 6 | Hu149-22 vH | EVQLVESGGGLVQPG |
| | | GSLKLSCAASGFTFN |
| | | TYAMNWVRQASGKGL |
| | | EWVGRIRSKSNFYAT |
| | | AYAASVKGRFTISRD |
| | | DSKNTAYLQMNSLKT |
| | | EDTAVYYCTRGREYG |
| | | SAYAMDYWGQGTLVT |
| | | VSS |
| 8 | Hu149-22 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLKYPLTFGG |
| | | GTKVELK |
| 11 | Hu149-23 vH | EVQLVESGGGLVQPG |
| | | GSLKLSCAASGFTFN |
| | | TYAMNWVRQAPGKGL |
| | | EWVGRIRSKSNFYAT |
| | | AYAASVKGRFTISRD |
| | | DSKNTAYLQMNSLKT |
| | | EDTAVYYCTRGREYG |
| | | SSYAMDYWGQGTLVT |
| | | VSS |
| 8 | Hu149-23 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLKYPLTFGG |
| | | GTKVELK |
| 12 | Hu149-24 vH | EVQLVESGGGLVQPG |
| | | GSLKLSCAASGFTFN |
| | | TYAMNWVRQAPGKGL |
| | | EWVGRIRSKSNFYAT |
| | | AYAASVKGRFTISRD |
| | | DSKNTAYLQMNSLKT |
| | | EDTAVYYCTRGKEYG |
| | | SSYAMDYWGQGTLVT |
| | | VSS |
| 8 | 149-24 vL | DIVMTQSPLSLPVTP |
| | | GEPASISCRSSKSLL |
| | | HSNANTYLYWFLQKP |
| | | GQSPQLLIYRMSNLA |
| | | SGVPDRFSGSGSGTD |
| | | FTLKISRVEAEDVGV |
| | | YYCMQHLKYPLTFGG |
| | | GTKVELK |
| 13 | Hu149-9 | TYAMN |
| 14 | HCDRs | RIRSKSNFYATAYAASVKG |
| 15 | | GRDYGSSYAMDY |
| 16 | Hu149-9 | RSSKSLLHSNANTYLY |
| 17 | LCDRs | RMSNLAS |
| 18 | | MQHLEYPLT |
| 13 | Hu149-11 | TYAMN |
| 14 | HCDRs | RIRSKSNFYATAYAASVKG |
| 19 | | GREYGSSYAMDY |
| 16 | Hu149-11 | RSSKSLLHSNANTYLY |
| 17 | LCDRs | RMSNLAS |
| 18 | | MQHLEYPLT |
| 13 | Hu149-12 | TYAMN |
| 14 | HCDRs | RIRSKSNFYATAYAASVKG |
| 20 | | GKEYGSSYAMDY |
| 16 | Hu149-12 | RSSKSLLHSNANTYLY |
| 17 | LCDRs | RMSNLAS |
| 18 | | MQHLEYPLT |
| 13 | Hu149-13 | TYAMN |
| 14 | HCDRs | RIRSKSNFYATAYAASVKG |
| 21 | | GREYGRSYAMDY |
| 16 | Hu149-13 | RSSKSLLHSNANTYLY |
| 17 | LCDRs | RMSNLAS |
| 18 | | MQHLEYPLT |
| 13 | Hu149-14 | TYAMN |
| 14 | HCDRs | RIRSKSNFYATAYAASVKG |
| 22 | | GREYGSAYAMDY |
| 16 | Hu149-14 | RSSKSLLHSNANTYLY |
| 17 | LCDRs | RMSNLAS |
| 18 | | MQHLEYPLT |
| 13 | Hu149-15 | TYAMN |
| 14 | HCDRs | RIRSKSNFYATAYAASVKG |
| 15 | | GRDYGSSYAMDY |
| 16 | Hu149-15 | RSSKSLLHSNANTYLY |
| 17 | LCDRs | RMSNLAS |
| 18 | | MQHLEYPLT |
| 13 | Hu149-16 | TYAMN |
| 14 | HCDRs | RIRSKSNFYATAYAASVKG |
| 15 | | GRDYGSSYAMDY |
| 16 | Hu149-16 | RSSKSLLHSNANTYLY |
| 17 | LCDRs | RMSNLAS |
| 23 | | MQHLKYPLT |
| 13 | Hu149-17 | TYAMN |
| 14 | HCDRs | RIRSKSNFYATAYAASVKG |
| 15 | | GRDYGSSYAMDY |
| 16 | Hu149-17 | RSSKSLLHSNANTYLY |
| 17 | LCDRs | RMSNLAS |
| 24 | | MQHLSYPLT |
| 13 | Hu149-18 | TYAMN |
| 14 | HCDRs | RIRSKSNFYATAYAASVKG |
| 15 | | GRDYGSSYAMDY |
| 16 | Hu149-18 | RSSKSLLHSNANTYLY |
| 17 | LCDRs | RMSNLAS |
| 25 | | MQHLRYPLT |
| 13 | Hu149-19 | TYAM |
| 14 | HCDRs | RIRSKSNFYATAYAASVKG |
| 19 | | GREYGSSYAMDY |
| 16 | Hu149-19 | RSSKSLLHSNANTYLY |
| 17 | LCDRs | RMSNLAS |
| 23 | | MQHLKYPLT |
| 13 | Hu149-20 | TYAMN |
| 14 | HCDRs | RIRSKSNFYATAYAASVKG |
| 20 | | GKEYGSSYAMDY |
| 16 | Hu149-20 | RSSKSLLHSNANTYLY |
| 17 | LCDRs | RMSNLAS |
| 23 | | MQHLKYPLT |
| 13 | Hu149-21 | TYAMN |
| 14 | HCDRs | RIRSKSNFYATAYAASVKG |
| 21 | | GREYGRSYAMDY |
| 16 | Hu149-21 | RSSKSLLHSNANTYLY |
| 17 | LCDRs | RMSNLAS |
| 23 | | MQHLKYPLT |
| 13 | Hu149-22 | TYAMN |
| 14 | HCDRs | RIRSKSNFYATAYAASVKG |
| 22 | | GREYGSAYAMDY |
| 16 | Hu149-22 | RSSKSLLHSNANTYLY |
| 17 | LCDRs | RMSNLAS |
| 23 | | MQHLKYPLT |
| 13 | Hu149-23 | TYAMN |
| 14 | HCDRs | RIRSKSNFYATAYAASVKG |
| 19 | | GREYGSSYAMDY |
| 16 | Hu149-23 | RSSKSLLHSNANTYLY |
| 17 | LCDRs | RMSNLAS |
| 23 | | MQHLKYPLT |
| 13 | Hu149-24 | TYAMN |
| 14 | HCDRs | RIRSKSNFYATAYAASVKG |
| 20 | | GKEYGSSYAMDY |
| 16 | Hu149-24 | RSSKSLLHSNANTYLY |
| 17 | LCDRs | RMSNLAS |
| 23 | | MQHLKYPLT |

TABLE 2
Amino acid sequence of variable region

| SEQ | | |
|---|---|---|
| ID | | |
| NO. | Name | Sequence |
| 26 | 191E12H8 HC | DVQLQESGPGLVKPSQSLSLTCSVT |
| | | GYSITSGYYWNWIRQFPGNKLEWMG |
| | | YISYDGSNNYNPSLKNRISITRDIS |
| | | KNQFFLKLNSVTTEDTATYYCARQI |
| | | GLRRGAMDYWGQGTSVTVSS |
| 27 | 191E12H8 LC | DIQMTQSSSYLSVSLGGRVTITCKA |
| | | SDHINTWLAWYQQKPGNAPRLLISG |
| | | STSLKAGVASRFSGSGSGKDYTLTI |
| | | TSLQTEDVATYYCQQYWGIPFTFGG |
| | | GTKLEIK |
| 28 | 234G9C12 HC | DVQLQESGPGLVKPSQSLSLTCSVT |
| | | GYSITSGYYWNWIRQFPGNKLEWMG |
| | | YTSYDGSNNYNPSFKNRISITRDIS |
| | | KNQFFLKLNSVTTEDTATYYCARQI |
| | | GLRRGAMDHWGQGTSVTVSS |
| 29 | 234G9C12 LC | DIQMTQSSSYLSVSLGGRVTITCKA |
| | | SDHINNWLAWYQQKPGNAPRLLISG |
| | | STSLRAGVASRFSGSGSGKDYTLTI |
| | | TSLQTEDVATYYCQQYWGIPFTFGG |
| | | GTKLEIK |
| 30 | 273H2G6 HC | DVQLQESGPGLVKPSQSLSLTCSVT |
| | | GYSITSGYYWNWIRQFPGNKLEWMG |
| | | YISYDGSNNYNPSLKNRISITRDIS |
| | | KNQFFLKLNSVTTEDTATYYCARQV |
| | | GLRRGAMDYWGQGTSVTVSS |
| 31 | 273H2G6 LC | DIQMTQSSSYLSVSLGGRVTITCKA |
| | | SDHINNWLAWYQQKPGNAPRLLISG |
| | | STSLKAGVASRFSGSGSGKDYTLSI |
| | | TSLQTEDVATYYCQQYWSIPYTFGG |
| | | GTKLEIK |
| 32 | 348E2D11 HC | QVQLVETGGGLVRPGNSLKLSCVTS |
| | | GFSFSNYRMHWLRQPPGKRLEWIAV |
| | | ITVKSDNYGANYAESVKGRFTISRD |
| | | DSKSSVYLQMNRLREEDTATYYCSS |
| | | LGRVAYWGQGTLVTVSA |
| 33 | 348E2D11 LC | DIVMTQSQKFMFTSVGDRVSVTCKA |
| | | SQNVDTNVVWYQQKPGQSPKALIYS |
| | | ASSRYSGVPDRFTGSGSGTDFTLTI |
| | | SNVQSEDLAEYFCQQHNSFPLTFGA |
| | | GTKLELI |
| 34 | 149F2C10 HC | EVQLVESGGGLVQPKGSLKLSCAAS |
| | | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 35 | 149F2C10 LC | DIVMTQAAPSVPVTPGESVSISCRS |
| | | SKSLLHSNGNTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 34 | 153D4G2 HC | EVQLVESGGGLVQPKGSLKLSCAAS |
| | | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 35 | 153D4G2 LC | DIVMTQAAPSVPVTPGESVSISCRS |
| | | SKSLLHSNGNTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 36 | 160D1E3 HC | EVQLVESGGGLVQPKGSLKLSCAAS |
| | | GFTFNTYAMNWVRQAPGKGLEWVAR |
| | | IRSKSNYYATYYADSVKDRFTISRD |
| | | DSQDMLYLQMNNLKTEDTAIYYCVR |
| | | GRDYGSSYAMDYWGQGTSVTVSS |
| 35 | 160D1E3 LC | DIVMTQAAPSVPVTPGESVSISCRS |
| | | SKSLLHSNGNTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 34 | 164G10D6 HC | EVQLVESGGGLVQPKGSLKLSCAAS |
| | | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 35 | 164G10D6 LC | DIVMTQAAPSVPVTPGESVSISCRS |
| | | SKSLLHSNGNTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 37 | 204A2G12 HC | DVQLQESGPGLVKPSQSLSLTCSVT |
| | | GYSITSGYYWNWIRQFPGNKLEWMG |
| | | YISYDGTNNYNPSLKNRISITRDTS |
| | | KNQFFLKLTSVTTEDTATYYCARQV |
| | | GLRRAWFAYWGQGTLVTVSA |
| 38 | 204A2G12 LC | DIQMTQSSSFLSVSLGGRVTITCKA |
| | | SDHINYWLAWYQQKPGNAPRLLISG |
| | | ATSLETGVPSRFSGSGSGKDYTLTI |
| | | TSLQNEDVATYYCHQYWSTPTFGSG |
| | | TKLEIK |
| 39 | 262C2G7 HC | DVHLQESGPGLVKPSQSLSLTCSVT |
| | | GYSITSGYYWNWIRQFPGNKLEWMG |
| | | YISYDGSNNYNPSLKNRISITRDIS |
| | | KNQFFLKLNSVTTEDTATYYCARQI |
| | | GLRRGAMDYWGQGTSVTVSS |
| 40 | 262C2G7 LC | DIQMTQSSSYLSVSLGGRVTITCKA |
| | | SDHINNWLAWYQQKPGNAPRLLISG |
| | | STSLKAGVASRFSGSGSGKDYTLTI |
| | | TSLQTEDVATYYCQQYWGIPFTFGG |
| | | GTKLEIK |
| 41 | 499B1A5 HC | EVQLVESGGGLVQPKGSLKLSCAAS |
| | | GFTFNTYAMNWVRQAPGKGLEWVAR |
| | | IRSKSNNYATYYADSVKDRFTISRD |
| | | DSQSMLYLQMNNLKTEDTAMYYCVR |
| | | HGRSGYLYAMDYWGQGTSVTVSS |
| 42 | 499B1A5 LC | DIVMTQAAFSNPVTLGTSASISCRS |
| | | SKSLLHSNGITYLYWYLQKPGQSPQ |
| | | LLIYQMSNLASGVPDRFSSSGSGTD |
| | | FTLRISRVEAEDVGVYYCAQSLELP |
| | | LTFGAGTKLELK |
| 34 | 149F2C10 N2 | EVQLVESGGGLVQPKGSLKLSCAAS |
| | 8Q HC | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 43 | 149F2C10 N2 | DIVMTQAAPSVPVTPGESVSISCRS |
| | 8QLC | SKSLLHSQGNTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 34 | 149F2C10 N2 | EVQLVESGGGLVQPKGSLKLSCAAS |
| | 8I HC | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 44 | 149F2C10 N2 | DIVMTQAAPSVPVTPGESVSISCRS |
| | 8ILC | SKSLLHSIGNTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 34 | 149F2C10_N2 | EVQLVESGGGLVQPKGSLKLSCAAS |
| | 8S HC | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 45 | 149F2C10 N2 | DIVMTQAAPSVPVTPGESVSISCRS |
| | 8S LC | SKSLLHSSGNTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 34 | 149F2C10 G2 | EVQLVESGGGLVQPKGSLKLSCAAS |
| | 9A HC | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 46 | 149F2C10 G2 | DIVMTQAAPSVPVTPGESVSISCRS |
| | 9A LC | SKSLLHSNANTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 34 | 149F2C10 G2 | EVQLVESGGGLVQPKGSLKLSCAAS |
| | 9I HC | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 47 | 149F2C10 G2 | DIVMTQAAPSVPVTPGESVSISCRS |
| | 9ILC | SKSLLHSNINTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 34 | 149F2C10 G2 | EVQLVESGGGLVQPKGSLKLSCAAS |
| | 9V HC | GFTFNTYAMNWVRQAPGKGLEWVAR |
| | | IRSKSNFYATYYADSVKDRFTISRD |
| | | DSQDMLYLQMNILKTEDTAIYYCVR |
| | | GRDYGSSYAMDYWGQGTSVTVSS |
| 48 | 149F2C10 G2 | DIVMTQAAPSVPVTPGESVSISCRS |
| | 9VLC | SKSLLHSNVNTYLYWFLQRPGQSPQ |
| | | LLIYRMSNLASGVPDRFSGSGSGTA |
| | | FTLRISRVEAEDVGVYYCMQHLEYP |
| | | LTFGAGTKLELK |
| 41 | 499B1A5 | EVQLVESGGGLVQPKGSLKLSCAAS |
| | L28(NtoS) HC | GFTFNTYAMNWVRQAPGKGLEWVAR |
| | | IRSKSNNYATYYADSVKDRFTISRD |
| | | DSQSMLYLQMNNLKTEDTAMYYCVR |
| | | HGRSGYLYAMDYWGQGTSVTVSS |
| 49 | 499B1A5 | DIVMTQAAFSNPVTLGTSASISCRS |
| | L28(NtoS) LC | SKSLLHSSGITYLYWYLQKPGQSPQ |
| | | LLIYQMSNLASGVPDRFSSSGSGTD |
| | | FTLRISRVEAEDVGVYYCAQSLELP |
| | | LTFGAGTKLELK |
| 41 | 499B1A5 | EVQLVESGGGLVQPKGSLKLSCAAS |
| | L28(NtoY) HC | GFTFNTYAMNWVRQAPGKGLEWVAR |
| | | IRSKSNNYATYYADSVKDRFTISRD |
| | | DSQSMLYLQMNNLKTEDTAMYYCVR |
| | | HGRSGYLYAMDYWGQGTSVTVSS |
| 50 | 499B1A5 | DIVMTQAAFSNPVTLGTSASISCRS |
| | L28(NtoY) LC | SKSLLHSYGITYLYWYLQKPGQSPQ |
| | | LLIYQMSNLASGVPDRFSSSGSGTD |
| | | FTLRISRVEAEDVGVYYCAQSLELP |
| | | LTFGAGTKLELK |
| 41 | 499B1A5 | EVQLVESGGGLVQPKGSLKLSCAAS |
| | L29(GtoA) HC | GFTFNTYAMNWVRQAPGKGLEWVAR |
| | | IRSKSNNYATYYADSVKDRFTISRD |
| | | DSQSMLYLQMNNLKTEDTAMYYCVR |
| | | HGRSGYLYAMDYWGQGTSVTVSS |
| 51 | 499B1A5 | DIVMTQAAFSNPVTLGTSASISCRS |
| | L29(GtoA) LC | SKSLLHSNAITYLYWYLQKPGQSPQ |
| | | LLIYQMSNLASGVPDRFSSSGSGTD |
| | | FTLRISRVEAEDVGVYYCAQSLELP |
| | | LTFGAGTKLELK |
| 41 | 499B1A5 | EVQLVESGGGLVQPKGSLKLSCAAS |
| | L51(MtoA) | GFTFNTYAMNWVRQAPGKGLEWVAR |
| | HC | IRSKSNNYATYYADSVKDRFTISRD |
| | | DSQSMLYLQMNNLKTEDTAMYYCVR |
| | | HGRSGYLYAMDYWGQGTSVTVSS |
| 52 | 499B1A5 | DIVMTQAAFSNPVTLGTSASISCRS |
| | L51(MtoA) LC | SKSLLHSNGITYLYWYLQKPGQSPQ |
| | | LLIYQASNLASGVPDRFSSSGSGTD |
| | | FTLRISRVEAEDVGVYYCAQSLELP |
| | | LTFGAGTKLELK |
| 41 | 499B1A5 | EVQLVESGGGLVQPKGSLKLSCAAS |
| | L51(MtoT) HC | GFTFNTYAMNWVRQAPGKGLEWVAR |
| | | IRSKSNNYATYYADSVKDRFTISRD |
| | | DSQSMLYLQMNNLKTEDTAMYYCVR |
| | | HGRSGYLYAMDYWGQGTSVTVSS |
| 53 | 499B1A5 | DIVMTQAAFSNPVTLGTSASISCRS |
| | L51(MtoT) LC | SKSLLHSNGITYLYWYLQKPGQSPQ |
| | | LLIYQTSNLASGVPDRFSSSGSGTD |
| | | FTLRISRVEAEDVGVYYCAQSLELP |
| | | LTFGAGTKLELK |
| 54 | 499B1A5 | EVQLVESGGGLVQPKGSLKLSCAAS |
| | H100D(MtoA) | GFTFNTYAMNWVRQAPGKGLEWVAR |
| | HC | IRSKSNNYATYYADSVKDRFTISRD |
| | | DSQSMLYLQMNNLKTEDTAMYYCVR |
| | | HGRSGYLYAADYWGQGTSVTVSS |
| 42 | 499B1A5 | DIVMTQAAFSNPVTLGTSASISCRS |
| | H100D(MtoA) | SKSLLHSNGITYLYWYLQKPGQSPQ |
| | LC | LLIYQMSNLASGVPDRFSSSGSGTD |
| | | FTLRISRVEAEDVGVYYCAQSLELP |
| | | LTFGAGTKLELK |
| 55 | 499B1A5 | EVQLVESGGGLVQPKGSLKLSCAAS |
| | H100D(MtoF) | GFTFNTYAMNWVRQAPGKGLEWVAR |
| | HC | IRSKSNNYATYYADSVKDRFTISRD |
| | | DSQSMLYLQMNNLKTEDTAMYYCVR |
| | | HGRSGYLYAFDYWGQGTSVTVSS |
| 42 | 499B1A5 | DIVMTQAAFSNPVTLGTSASISCRS |
| | H100D(MtoF) | SKSLLHSNGITYLYWYLQKPGQSPQ |
| | LC | LLIYQMSNLASGVPDRFSSSGSGTD |
| | | FTLRISRVEAEDVGVYYCAQSLELP |
| | | LTFGAGTKLELK |
| 56 | Hu149-1 HC | EVQLVESGGGLVQPGGSLKLSCAAS |
| | | GFTFNTYAMNWVRQASGKGLEWVGR |
| | | IRSKSNFYATYYAASVKGRFTISRD |
| | | DSKNTAYLQMNSLKTEDTAVYYCVR |
| | | GRDYGSSYAMDYWGQGTLVTVSS |
| 2 | Hu149-1 LC | DIVMTQSPLSLPVTPGEPASISCRS |
| | | SKSLLHSNANTYLYWFLQKPGQSPQ |
| | | LLIYRMSNLASGVPDRFSGSGSGTD |
| | | FTLKISRVEAEDVGVYYCMQHLEYP |
| | | LTFGGGTKVELK |
| 57 | Hu149-2 HC | EVQLVESGGGLVQPGGSLKLSCAAS |
| | | GFTFNTYAMHWVRQASGKGLEWVGR |
| | | IRSKSNFYATYYAASVKGRFTISRD |
| | | DSKNTAYLQMNSLKTEDTAVYYCVR |
| | | GRDYGSSYAMDYWGQGTLVTVSS |
| 2 | Hu149-2 LC | DIVMTQSPLSLPVTPGEPASISCRS |
| | | SKSLLHSNANTYLYWFLQKPGQSPQ |
| | | LLIYRMSNLASGVPDRFSGSGSGTD |
| | | FTLKISRVEAEDVGVYYCMQHLEYP |
| | | LTFGGGTKVELK |
| 58 | Hu149-3 HC | EVQLVESGGGLVQPGGSLKLSCAAS |
| | | GFTFNTYAMNWVRQASGKGLEWVGR |
| | | IRSKSNFYATAYAASVKGRFTISRD |
| | | DSKNTAYLQMNSLKTEDTAVYYCVR |
| | | GRDYGSSYAMDYWGQGTLVTVSS |
| 2 | Hu149-3 LC | DIVMTQSPLSLPVTPGEPASISCRS |
| | | SKSLLHSNANTYLYWFLQKPGQSPQ |
| | | LLIYRMSNLASGVPDRFSGSGSGTD |
| | | FTLKISRVEAEDVGVYYCMQHLEYP |
| | | LTFGGGTKVELK |
| 59 | Hu149-4 HC | EVQLVESGGGLVQPGGSLKLSCAAS |
| | | GFTFNTYAMNWVRQASGKGLEWVGR |
| | | IRSKSNFYATYYAASVKGRFTISRD |
| | | DSKNTAYLQMNSLKTEDTAVYYCTR |
| | | GRDYGSSYAMDYWGQGTLVTVSS |
| 2 | Hu149-4 LC | DIVMTQSPLSLPVTPGEPASISCRS |
| | | SKSLLHSNANTYLYWFLQKPGQSPQ |
| | | LLIYRMSNLASGVPDRFSGSGSGTD |
| | | FTLKISRVEAEDVGVYYCMQHLEYP |
| | | LTFGGGTKVELK |
| 56 | Hu149-5 HC | EVQLVESGGGLVQPGGSLKLSCAAS |
| | | GFTFNTYAMNWVRQASGKGLEWVGR |
| | | IRSKSNFYATYYAASVKGRFTISRD |
| | | DSKNTAYLQMNSLKTEDTAVYYCVR |
| | | GRDYGSSYAMDYWGQGTLVTVSS |
| 60 | Hu149-5 LC | DIVMTQSPLSLPVTPGEPASISCRS |
| | | SKSLLHSNANTYLYWYLQKPGQSPQ |
| | | LLIYRMSNLASGVPDRFSGSGSGTD |
| | | FTLKISRVEAEDVGVYYCMQHLEYP |
| | | LTFGGGTKVELK |
| 57 | Hu149-6 HC | EVQLVESGGGLVQPGGSLKLSCAAS |
| | | GFTFNTYAMHWVRQASGKGLEWVGR |
| | | IRSKSNFYATYYAASVKGRFTISRD |
| | | DSKNTAYLQMNSLKTEDTAVYYCVR |
| | | GRDYGSSYAMDYWGQGTLVTVSS |
| 60 | Hu149-6 LC | DIVMTQSPLSLPVTPGEPASISCRS |
| | | SKSLLHSNANTYLYWYLQKPGQSPQ |
| | | LLIYRMSNLASGVPDRFSGSGSGTD |
| | | FTLKISRVEAEDVGVYYCMQHLEYP |
| | | LTFGGGTKVELK |
| 58 | Hu149-7 HC | EVQLVESGGGLVQPGGSLKLSCAAS |
| | | GFTFNTYAMNWVRQASGKGLEWVGR |
| | | IRSKSNFYATAYAASVKGRFTISRD |
| | | DSKNTAYLQMNSLKTEDTAVYYCVR |
| | | GRDYGSSYAMDYWGQGTLVTVSS |
| 60 | Hu149-7 LC | DIVMTQSPLSLPVTPGEPASISCRS |
| | | SKSLLHSNANTYLYWYLQKPGQSPQ |
| | | LLIYRMSNLASGVPDRFSGSGSGTD |
| | | FTLKISRVEAEDVGVYYCMQHLEYP |
| | | LTFGGGTKVELK |
| 59 | Hu149-8 HC | EVQLVESGGGLVQPGGSLKLSCAAS |
| | | GFTFNTYAMNWVRQASGKGLEWVGR |
| | | IRSKSNFYATYYAASVKGRFTISRD |
| | | DSKNTAYLQMNSLKTEDTAVYYCTR |
| | | GRDYGSSYAMDYWGQGTLVTVSS |
| 60 | Hu149-8 LC | DIVMTQSPLSLPVTPGEPASISCRS |
| | | SKSLLHSNANTYLYWYLQKPGQSPQ |
| | | LLIYRMSNLASGVPDRFSGSGSGTD |
| | | FTLKISRVEAEDVGVYYCMQHLEYP |
| | | LTFGGGTKVELK |
| 61 | Hu348-1 HC | EVQLVESGGGLVQPGGSLKLSCATS |
| | | GFSFSNYRMHWLRQASGKGLEWVGV |
| | | ITVKSDNYGANYAASVKGRFTISRD |
| | | DSKSSVYLQMNSLKTEDTAVYYCSS |
| | | LGRVAYWGQGTLVTVSS |
| 62 | Hu348-1 LC | DIQMTQSPSSVSASVGDRVTITCKA |
| | | SQNVDTNVVWYQQKPGQSPKALIYS |
| | | ASSRYSGVPDRFSGSGSGTDFTLTI |
| | | SSLQPEDFATYYCQQHNSFPLTFGQ |
| | | GTKLEIK |
| 61 | Hu348-2 HC | EVQLVESGGGLVQPGGSLKLSCATS |
| | | GFSFSNYRMHWLRQASGKGLEWVGV |
| | | ITVKSDNYGANYAASVKGRFTISRD |
| | | DSKSSVYLQMNSLKTEDTAVYYCSS |
| | | LGRVAYWGQGTLVTVSS |
| 63 | Hu348-2 LC | DIQMTQSPSSVSASVGDRVTITCKA |
| | | SQNVDTNVVWYQQKPGKAPKALIYS |
| | | ASSRYSGVPDRFSGSGSGTDFTLTI |
| | | SSLQPEDFATYYCQQHNSFPLTFGQ |
| | | GTKLEIK |
| 61 | Hu348-3 HC | EVQLVESGGGLVQPGGSLKLSCATS |
| | | GFSFSNYRMHWLRQASGKGLEWVGV |
| | | ITVKSDNYGANYAASVKGRFTISRD |
| | | DSKSSVYLQMNSLKTEDTAVYYCSS |
| | | LGRVAYWGQGTLVTVSS |
| 64 | Hu348-3 LC | DIQMTQSPSSVSASVGDRVTITCKA |
| | | SQNVDTNVVWYQQKPGQSPKLLIYS |
| | | ASSRYSGVPDRFSGSGSGTDFTLTI |
| | | SSLQPEDFATYYCQQHNSFPLTFGQ |
| | | GTKLEIK |
| 61 | Hu348-4 HC | EVQLVESGGGLVQPGGSLKLSCATS |
| | | GFSFSNYRMHWLRQASGKGLEWVGV |
| | | ITVKSDNYGANYAASVKGRFTISRD |
| | | DSKSSVYLQMNSLKTEDTAVYYCSS |
| | | LGRVAYWGQGTLVTVSS |
| 65 | Hu348-4 LC | DIQMTQSPSSVSASVGDRVTITCKA |
| | | SQNVDTNVVWYQQKPGQSPKALIYS |
| | | ASSRYSGVPSRFSGSGSGTDFTLTI |
| | | SSLQPEDFATYYCQQHNSFPLTFGQ |
| | | GTKLEIK |
| 66 | Hu348-5 HC | EVQLVESGGGLVQPGGSLKLSCAAS |
| | | GFSFSNYAMHWLRQASGKGLEWVGV |
| | | ITVKSDNYGANYAASVKGRFTISRD |
| | | DSKSSVYLQMNSLKTEDTAVYYCSS |
| | | LGRVAYWGQGTLVTVSS |
| 62 | Hu348-5 LC | DIQMTQSPSSVSASVGDRVTITCKA |
| | | SQNVDTNVVWYQQKPGQSPKALIYS |
| | | ASSRYSGVPDRFSGSGSGTDFTLTI |
| | | SSLQPEDFATYYCQQHNSFPLTFGQ |
| | | GTKLEIK |
| 67 | Hu348-6 HC | EVQLVESGGGLVQPGGSLKLSCATS |
| | | GFSFSNYRMHWVRQASGKGLEWVGV |
| | | ITVKSDNYGANYAASVKGRFTISRD |
| | | DSKSSVYLQMNSLKTEDTAVYYCSS |
| | | LGRVAYWGQGTLVTVSS |
| 62 | Hu348-6 LC | DIQMTQSPSSVSASVGDRVTITCKA |
| | | SQNVDTNVVWYQQKPGQSPKALIYS |
| | | ASSRYSGVPDRFSGSGSGTDFTLTI |
| | | SSLQPEDFATYYCQQHNSFPLTFGQ |
| | | GTKLEIK |
| 68 | Hu348-7 HC | EVQLVESGGGLVQPGGSLKLSCATS |
| | | GFSFSNYRMHWLRQASGKGLEWVGR |
| | | ITVKSDNYGTAYAASVKGRFTISRD |
| | | DSKSSVYLQMNSLKTEDTAVYYCSS |
| | | LGRVAYWGQGTLVTVSS |
| 62 | Hu348-7 LC | DIQMTQSPSSVSASVGDRVTITCKA |
| | | SQNVDTNVVWYQQKPGQSPKALIYS |
| | | ASSRYSGVPDRFSGSGSGTDFTLTI |
| | | SSLQPEDFATYYCQQHNSFPLTFGQ |
| | | GTKLEIK |
| 69 | Hu348-8 HC | EVQLVESGGGLVQPGGSLKLSCATS |
| | | GFSFSNYRMHWLRQASGKGLEWVGV |
| | | ITVKSDNYGANYAASVKGRFTISRD |
| | | DSKNTAYLQMNSLKTEDTAVYYCSS |
| | | LGRVAYWGQGTLVTVSS |
| 62 | Hu348-8 LC | DIQMTQSPSSVSASVGDRVTITCKA |
| | | SQNVDTNVVWYQQKPGQSPKALIYS |
| | | ASSRYSGVPDRFSGSGSGTDFTLTI |
| | | SSLQPEDFATYYCQQHNSFPLTFGQ |
| | | GTKLEIK |
| 70 | Hu348-9 HC | EVQLVESGGGLVQPGGSLKLSCATS |
| | | GFSFSNYRMHWLRQASGKGLEWVGV |
| | | ITVKSDNYGANYAASVKGRFTISRD |
| | | DSKSSVYLQMNSLKTEDTAVYYCTS |
| | | LGRVAYWGQGTLVTVSS |
| 62 | Hu348-9 LC | DIQMTQSPSSVSASVGDRVTITCKA |
| | | SQNVDTNVVWYQQKPGQSPKALIYS |
| | | ASSRYSGVPDRFSGSGSGTDFTLTI |
| | | SSLQPEDFATYYCQQHNSFPLTFGQ |
| | | GTKLEIK |

TABLE 3
Amino acid sequence of CDR definition
by Kabat naming system

| No. | Name | CDRs (Kabat) |
|---|---|---|
| 71 | 191E12H8 HC | SGYYWN |
| 72 | | YISYDGSNNYNPSLKN |
| 73 | | QIGLRRGAMDY |
| 74 | 191E12H8 LC | KASDHINTWLA |
| 75 | | GSTSLKA |
| 76 | | QQYWGIPFT |
| 71 | 234G9C12 HC | SGYYWN |
| 77 | | YTSYDGSNNYNPSFKN |
| 78 | | QIGLRRGAMDH |
| 79 | 234G9C12 LC | KASDHINNWLA |
| 80 | | GSTSLRA |
| 76 | | QQYWGIPFT |
| 71 | 273H2G6 HC | SGYYWN |
| 72 | | YISYDGSNNYNPSLKN |
| 81 | | QVGLRRGAMDY |
| 79 | 273H2G6 LC | KASDHINNWLA |
| 75 | | GSTSLKA |
| 82 | | QQYWSIPYT |
| 83 | 348E2D11 HC | NYRMH |
| 84 | | VITVKSDNYGANYAESVKG |
| 85 | | LGRVAY |
| 86 | 348E2D11 LC | KASQNVDTNVV |
| 87 | | SASSRYS |
| 88 | | QQHNSFPLT |
| 13 | 149F2C10 HC | TYAMN |
| 89 | | RIRSKSNFYATYYADSVKD |
| 15 | | GRDYGSSYAMDY |
| 90 | 149F2C10 LC | RSSKSLLHSNGNTYLY |
| 17 | | RMSNLAS |
| 18 | | MQHLEYPLT |
| 13 | 153D4G2 HC | TYAMN |
| 89 | | RIRSKSNFYATYYADSVKD |
| 15 | | GRDYGSSYAMDY |
| 90 | 153D4G2 LC | RSSKSLLHSNGNTYLY |
| 17 | | RMSNLAS |
| 18 | | MQHLEYPLT |
| 13 | 160D1E3 HC | TYAMN |
| 91 | | RIRSKSNYYATYYADSVKD |
| 15 | | GRDYGSSYAMDY |
| 90 | 160D1E3 LC | RSSKSLLHSNGNTYLY |
| 17 | | RMSNLAS |
| 18 | | MQHLEYPLT |
| 13 | 164G10D6 HC | TYAMN |
| 89 | | RIRSKSNFYATYYADSVKD |
| 15 | | GRDYGSSYAMDY |
| 90 | 164G10D6 LC | RSSKSLLHSNGNTYLY |
| 17 | | RMSNLAS |
| 18 | | MQHLEYPLT |
| 71 | 204A2G12 HC | SGYYWN |
| 92 | | YISYDGTNNYNPSLKN |
| 93 | | QVGLRRAWFAY |
| 94 | 204A2G12 LC | KASDHINYWLA |
| 95 | | GATSLET |
| 96 | | HQYWSTPT |
| 71 | 262C2G7 HC | SGYYWN |
| 72 | | YISYDGSNNYNPSLKN |
| 73 | | QIGLRRGAMDY |
| 79 | 262C2G7 LC | KASDHINNWLA |
| 75 | | GSTSLKA |
| 76 | | QQYWGIPFT |
| 13 | 499B1A5 HC | TYAMN |
| 97 | | RIRSKSNNYATYYADSVKD |
| 98 | | HGRSGYLYAMDY |
| 99 | 499B1A5 LC | RSSKSLLHSNGITYLY |
| 100 | | QMSNLAS |
| 101 | | AQSLELPLT |
| 13 | 149F2C10_N28Q HC | TYAMN |
| 89 | | RIRSKSNFYATYYADSVKD |
| 15 | | GRDYGSSYAMDY |
| 102 | 149F2C10_N28Q LC | RSSKSLLHSQGNTYLY |
| 17 | | RMSNLAS |
| 18 | | MQHLEYPLT |
| 13 | 149F2C10_N28I HC | TYAMN |
| 89 | | RIRSKSNFYATYYADSVKD |
| 15 | | GRDYGSSYAMDY |
| 103 | 149F2C10_N28ILC | RSSKSLLHSIGNTYLY |
| 17 | | RMSNLAS |
| 18 | | MQHLEYPLT |
| 13 | 149F2C10_N28S HC | TYAMN |
| 89 | | RIRSKSNFYATYYADSVKD |
| 15 | | GRDYGSSYAMDY |
| 104 | 149F2C10_N28S LC | RSSKSLLHSSGNTYLY |
| 17 | | RMSNLAS |
| 18 | | MQHLEYPLT |
| 13 | 149F2C10_G29A HC | TYAMN |
| 89 | | RIRSKSNFYATYYADSVKD |
| 15 | | GRDYGSSYAMDY |
| 16 | 149F2C10_G29A LC | RSSKSLLHSNANTYLY |
| 17 | | RMSNLAS |
| 18 | | MQHLEYPLT |
| 13 | 149F2C10_G29I HC | TYAMN |
| 89 | | RIRSKSNFYATYYADSVKD |
| 15 | | GRDYGSSYAMDY |
| 105 | 149F2C10_G29I LC | RSSKSLLHSNINTYLY |
| 17 | | RMSNLAS |
| 18 | | MQHLEYPLT |
| 13 | 149F2C10_G29V HC | TYAMN |
| 89 | | RIRSKSNFYATYYADSVKD |
| 15 | | GRDYGSSYAMDY |
| 106 | 149F2C10_G29V LC | RSSKSLLHSNVNTYLY |
| 17 | | RMSNLAS |
| 18 | | MQHLEYPLT |
| 13 | 499B1A5_L28(NtoS) HC | TYAMN |
| 97 | | RIRSKSNNYATYYADSVKD |
| 98 | | HGRSGYLYAMDY |
| 107 | 499B1A5_L28(NtoS) LC | RSSKSLLHSSGITYLY |
| 100 | | QMSNLAS |
| 101 | | AQSLELPLT |
| 13 | 499B1A5__L28(NtoY) | TYAMN |
| 97 | HC | RIRSKSNNYATYYADSVKD |
| 98 | | HGRSGYLYAMDY |
| 108 | 499B1A5_L28(NtoY) | RSSKSLLHSYGITYLY |
| 100 | LC | QMSNLAS |
| 101 | | AQSLELPLT |
| 13 | 499B1A5_L29(GtoA) | TYAMN |
| 97 | HC | RIRSKSNNYATYYADSVKD |
| 98 | | HGRSGYLYAMDY |
| 109 | 499B1A5_L29(GtoA) | RSSKSLLHSNAITYLY |
| 100 | LC | QMSNLAS |
| 101 | | AQSLELPLT |
| 13 | 499B1A5_L51(MtoA) | TYAMN |
| 97 | HC | RIRSKSNNYATYYADSVKD |
| 98 | | HGRSGYLYAMDY |
| 99 | 499B1A5_L51(MtoA) | RSSKSLLHSNGITYLY |
| 110 | LC | QASNLAS |
| 101 | | AQSLELPLT |
| 13 | 499B1A5_L51(MtoT) | TYAMN |
| 97 | HC | RIRSKSNNYATYYADSVKD |
| 98 | | HGRSGYLYAMDY |
| 99 | 499B1A5_L51(MtoT) | RSSKSLLHSNGITYLY |
| 111 | LC | QTSNLAS |
| 101 | | AQSLELPLT |
| 13 | 499B1A5_H100D(MtoA) | TYAMN |
| 97 | HC | RIRSKSNNYATYYADSVKD |
| 112 | | HGRSGYLYAADY |
| 99 | 499B1A5_H100D(MtoA) | RSSKSLLHSNGITYLY |
| 100 | LC | QMSNLAS |
| 101 | | AQSLELPLT |
| 13 | 499B1A5_H100D(MtoF) | TYAMN |
| 97 | HC | RIRSKSNNYATYYADSVKD |
| 113 | | HGRSGYLYAFDY |
| 99 | 499B1A5_H100D(MtoF) | RSSKSLLHSNGITYLY |
| 100 | LC | QMSNLAS |
| 101 | | AQSLELPLT |
| 13 | Hu149-1 HC | TYAMN |
| 114 | | RIRSKSNFYATYYAASVKG |
| 15 | | GRDYGSSYAMDY |
| 16 | Hu149-1 LC | RSSKSLLHSNANTYLY |
| 17 | | RMSNLAS |
| 18 | | MQHLEYPLT |
| 115 | Hu149-2 HC | TYAMH |
| 114 | | RIRSKSNFYATYYAASVKG |
| 15 | | GRDYGSSYAMDY |
| 16 | Hu149-2 LC | RSSKSLLHSNANTYLY |
| 17 | | RMSNLAS |
| 18 | | MQHLEYPLT |
| 13 | Hu149-3 HC | TYAMN |
| 14 | | RIRSKSNFYATAYAASVKG |
| 15 | | GRDYGSSYAMDY |
| 16 | Hu149-3 LC | RSSKSLLHSNANTYLY |
| 17 | | RMSNLAS |
| 18 | | MQHLEYPLT |
| 13 | Hu149-4 HC | TYAMN |
| 114 | | RIRSKSNFYATYYAASVKG |
| 15 | | GRDYGSSYAMDY |
| 16 | Hu149-4 LC | RSSKSLLHSNANTYLY |
| 17 | | RMSNLAS |
| 18 | | MQHLEYPLT |
| 13 | Hu149-5 HC | TYAMN |
| 114 | | RIRSKSNFYATYYAASVKG |
| 15 | | GRDYGSSYAMDY |
| 16 | Hu149-5 LC | RSSKSLLHSNANTYLY |
| 17 | | RMSNLAS |
| 18 | | MQHLEYPLT |
| 115 | Hu149-6 HC | TYAMH |
| 114 | | RIRSKSNFYATYYAASVKG |
| 15 | | GRDYGSSYAMDY |
| 16 | Hu149-6 LC | RSSKSLLHSNANTYLY |
| 17 | | RMSNLAS |
| 18 | | MQHLEYPLT |
| 13 | Hu149-7 HC | TYAMN |
| 14 | | RIRSKSNFYATAYAASVKG |
| 15 | | GRDYGSSYAMDY |
| 16 | Hu149-7 LC | RSSKSLLHSNANTYLY |
| 17 | | RMSNLAS |
| 18 | | MQHLEYPLT |
| 13 | Hu149-8 HC | TYAMN |
| 114 | | RIRSKSNFYATYYAASVKG |
| 15 | | GRDYGSSYAMDY |
| 16 | Hu149-8 LC | RSSKSLLHSNANTYLY |
| 17 | | RMSNLAS |
| 18 | | MQHLEYPLT |
| 83 | Hu348-1 HC | NYRMH |
| 116 | | VITVKSDNYGANYAASVKG |
| 85 | | LGRVAY |
| 86 | Hu348-1 LC | KASQNVDTNVV |
| 87 | | SASSRYS |
| 88 | | QQHNSFPLT |
| 83 | Hu348-2 HC | NYRMH |
| 116 | | VITVKSDNYGANYAASVKG |
| 85 | | LGRVAY |
| 86 | Hu348-2 LC | KASQNVDTNVV |
| 87 | | SASSRYS |
| 88 | | QQHNSFPLT |
| 83 | Hu348-3 HC | NYRMH |
| 116 | | VITVKSDNYGANYAASVKG |
| 85 | | LGRVAY |
| 86 | Hu348-3 LC | KASQNVDTNVV |
| 87 | | SASSRYS |
| 88 | | QQHNSFPLT |
| 83 | Hu348-4 HC | NYRMH |
| 116 | | VITVKSDNYGANYAASVKG |
| 85 | | LGRVAY |
| 86 | Hu348-4 LC | KASQNVDTNVV |
| 87 | | SASSRYS |
| 88 | | QQHNSFPLT |
| 117 | Hu348-5 HC | NYAMH |
| 116 | | VITVKSDNYGANYAASVKG |
| 85 | | LGRVAY |
| 86 | Hu348-5 LC | KASQNVDTNVV |
| 87 | | SASSRYS |
| 88 | | QQHNSFPLT |
| 83 | Hu348-6 HC | NYRMH |
| 116 | | VITVKSDNYGANYAASVKG |
| 85 | | LGRVAY |
| 86 | Hu348-6 LC | KASQNVDTNVV |
| 87 | | SASSRYS |
| 88 | | QQHNSFPLT |
| 83 | Hu348-7 HC | NYRMH |
| 118 | | RITVKSDNYGTAYAASVKG |
| 85 | | LGRVAY |
| 86 | Hu348-7 LC | KASQNVDTNVV |
| 87 | | SASSRYS |
| 88 | | QQHNSFPLT |
| 83 | Hu348-8 HC | NYRMH |
| 116 | | VITVKSDNYGANYAASVKG |
| 85 | | LGRVAY |
| 86 | Hu348-8 LC | KASQNVDTNVV |
| 87 | | SASSRYS |
| 88 | | QQHNSFPLT |
| 83 | Hu348-9 HC | NYRMH |
| 116 | | VITVKSDNYGANYAASVKG |
| 85 | | LGRVAY |
| 86 | Hu348-9 LC | KASQNVDTNVV |
| 87 | | SASSRYS |
| 88 | | QQHNSFPLT |

In some embodiments, a subject antigen binding unit is a monoclonal antigen binding unit, a polyclonal antigen binding unit, a humanized antigen binding unit, a chimeric antigen binding unit, a monovalent antigen binding unit, a multivalent antigen binding unit, a bispecific antigen binding unit, or any combination thereof. The antigen binding units can adopt a variety of formats, including but not limited to scFv, Fab′, a single chain Fab (scFab′), a Fd or a F(ab′)₂, sFC, Fv, and ccFv. Such antibody binding units can be generated from whole immunoglobulins by ricin, pepsin, papain, or other protease cleavage.

In addition, antigen binding units can be designed utilizing recombinant immunoglobulin techniques. For instance, “Fv” immunoglobulins may be produced by linking a variable light chain region to a variable heavy chain region via a peptide linker. For example, a peptide linker can be poly-glycine or another sequence which does not form an alpha helix or beta sheet motif. Fvs can also be made which comprise stabilizing disulfide bonds between the VH and VLregions, as described in U.S. Pat. No. 6,147,203, incorporated fully herein by reference. Any of these antigen binding unites can be utilized. In some aspects, an antigen binding unit can be a whole immunoglobulin having two light chains paired with two heavy chains.

Antigen-binding units can be heteromultimers comprising a light-chain polypeptide and a heavy-chain polypeptide. Examples of an antigen binding unit include but are not limited to (i) a ccFv fragment stabilized by the heterodimerization sequences disclosed U.S. Pat. No. 6,833,441, incorporated herein in its entirety; (ii) any other monovalent and multivalent molecules comprising at least one ccFv fragment as described herein; (iii) a Fab fragment consisting of the VL, VH, CL and CH1 domains; (iv) an Fd fragment consisting of the VH and CH1 domains; (v) an Fv fragment consisting of the VL and VH domains of a single arm of an antibody; (vi) an F(ab′)₂ fragment, a bivalent fragment comprising two Fab fragments linked by a disulfide bridge at the hinge region; and (vii) a diabody.

Polyclonal antibodies can be raised by a standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal antigen binding units or antibodies can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, incorporated fully herein by reference. Such antibodies can be referred to as human xenogenic antibodies.

Alternatively, antigen binding units can be produced from phage libraries containing human variable regions. See U.S. Pat. No. 6,174,708, incorporated fully herein by reference.

In some aspects of any of the embodiments disclosed herein, an antigen binding unit is produced by a hybridoma. For example, an antigen binding unit disclosed herein can be produced by a hybridoma selected form the group consisting of hybridomas expressing one of the antigen binding units listed in Table 1.

For monoclonal antigen binding units or monoclonal antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the antigen binding unit may be produced by genetic engineering. Humanized, chimeric, or xenogeneic human antigen binding units, which produce less of an immune response when administered to humans, are contemplated.

Antigen binding units disclosed herein can have a reduced propensity to induce an undesired immune response in humans, for example, anaphylactic shock, and can also exhibit a reduced propensity for priming an immune response which would prevent repeated dosage with the antibody therapeutic or imaging agent (e.g., the human-anti-murine-antibody “HAMA” response). Such antigen binding units include, but are not limited to, humanized, chimeric, or xenogenic human antigen binding units.

Chimeric antigen binding units or chimeric antibodies can be made, for example, by recombinant means by combining the murine variable light and heavy chain regions (VL and VH), obtained from a murine (or other animal-derived) hybridoma clone, with the human constant light and heavy chain regions, in order to produce an antibody with predominantly human domains. The production of such chimeric antibodies is well known in the art and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body.

In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin, such as human IgG1.

Humanized antibodies can be engineered to contain human-like immunoglobulin domains and incorporate only the complementarity-determining regions of the animal-derived antibody.

This can be accomplished by carefully examining the sequence of the hyper-variable loops of the variable regions of a monoclonal antigen binding unit or monoclonal antibody and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. HuAbs with different variable (V) regions are contemplated. It is within the skill of one in the art to determine suitable V region sequences and to optimize these sequences. Methods for obtaining antibodies with reduced immunogenicity are also described in U.S. Pat. No. 5,270,202 and EP 699,755.

Humanized antibodies can be prepared by a process of analysis of the parental sequences and various conceptual humanized products using three dimensional models of the parental and humanized sequences. Three-dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions is selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

As noted above, an antigen binding units can be either “monovalent” or “multivalent.” Whereas the former has one binding site per antigen-binding unit, the latter contains multiple binding sites capable of binding to more than one antigen of the same or different kind. Depending on the number of binding sites, antigen binding units may be bivalent (having two antigen-binding sites), trivalent (having three antigen-binding sites), tetravalent (having four antigen-binding sites), and so on.

Multivalent antigen binding units can be further classified on the basis of their binding specificities. A “monospecific” antigen binding unit is a molecule capable of binding to one or more antigens of the same kind. A “multispecific” antigen binding unit is a molecule having binding specificities for at least two different antigens. While such molecules normally will only bind two distinct antigens (i.e. bispecific antigen binding units), antibodies with additional specificities such as trispecific antibodies are encompassed by this expression when used herein. This disclosure further provides multispecific antigen binding units. Multispecific antigen binding units are multivalent molecules capable of binding to at least two distinct antigens. Preferred multispecific antigen binding units are bispecific and trispecific molecules exhibiting binding specificities to two and three distinct antigens, respectively.

In some embodiments, an antigen binding unit is a bispecific antigen binding unit, wherein the antigen binding unit specifically binds to CCR8 and a second antigen. In some examples, the second antigen is binds to a CCR8 from a different species. For example, the bispecific antigen binding unit may bind a human CCR8 and a cynomolgus CCR8. Alternatively, the bispecific antigen binding unit may encompass a binding property whereby it binds for example, a human CCR8, and a blocking property whereby it prevents for example, binding of CCL1 to human CCR8. Other suitable second antigens include, though are not limited to, a tumor cell antigen, an immune cell antigen, a cytotoxic trigger molecule, a toxin a fibrinolytic agent, a cell surface receptor, infectious disease target, a vaccine adjuvant, a diagnostic agent, a detection molecule, and a reporter molecule.

### Polynucleotides and Vectors

In some embodiments, isolated nucleic acids encoding any of the antigen binding units disclosed herein are provided. In another embodiment, vectors comprising a nucleic acid sequence encoding any antigen binding unit disclosed herein are provided. In some embodiments, isolated nucleic acids that encode a light-chain CDR and a heavy-chain CDR of an antigen binding unit disclosed herein are provided.

The subject antigen binding units can be prepared by recombinant DNA technology, synthetic chemistry techniques, or a combination thereof. For instance, sequences encoding the desired components of the antigen binding units, including light chain CDRs and heavy chain CDRs are typically assembled cloned into an expression vector using standard molecular techniques know in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector comprising the antigen binding unit of interest.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection (atcc.org), which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding antigen binding units can also be modified, for example, by substituting the coding sequence for human heavy and light chain constant regions in place of the homologous non-human sequences. In that manner, chimeric antibodies are prepared that retain the binding specificity of the original antigen binding unit.

It is also understood that the polynucleotides include those coding for functional equivalents and fragments thereof of the exemplified polypeptides. Functionally equivalent polypeptides include those that enhance, decrease or not significantly affect properties of the polypeptides encoded thereby. Functional equivalents may be polypeptides having conservative amino acid substitutions, analogs including fusions, and mutants.

Due to the degeneracy of the genetic code, there can be considerable variation in nucleotides of an antigen binding unit coding sequence, as well as sequences suitable for construction of the polynucleotide and vectors. Sequence variants may have modified DNA or amino acid sequences, one or more substitutions, deletions, or additions, the net effect of which is to retain the desired antigen-binding activity. For instance, various substitutions can be made in the coding region that either do not alter the amino acids encoded or result in conservative changes. These substitutions are encompassed by the disclosure herein. Conservative amino acid substitutions include substitutions within the following groups: glycine, alanine; valine, isoleucine, leucine; aspartic acid, glutamic acid; asparagine, glutamine; serine, threonine; lysine, arginine; and phenylalanine, tyrosine. While conservative substitutions do effectively change one or more amino acid residues contained in the polypeptide to be produced, the substitutions are not expected to interfere with the antigen-binding activity of the resulting antigen binding units to be produced. Nucleotide substitutions that do not alter the amino acid residues encoded are useful for optimizing gene expression in different systems. Suitable substitutions are known to those of skill in the art and are made, for instance, to reflect preferred codon usage in the expression systems.

Where desired, the recombinant polynucleotides may comprise heterologous sequences that facilitate detection of the expression and purification of the gene product. Examples of such sequences are known in the art and include those encoding reporter proteins such as β-galactosidase, β-lactamase, chloramphenicol acetyltransferase (CAT), luciferase, green fluorescent protein (GFP) and their derivatives. Other heterologous sequences that facilitate purification may code for epitopes such as Myc, HA (derived from influenza virus hemagglutinin), His-6, FLAG, or the Fc portion of immunoglobulin, glutathione S-transferase (GST), and maltose-binding protein (MBP).

Polynucleotides disclosed herein can be conjugated to a variety of chemically functional moieties described above. Commonly employed moieties include labels capable of producing a detectable signal, signal peptides, agents that enhance immunologic reactivity, agents that facilitate coupling to a solid support, vaccine carriers, bioresponse modifiers, paramagnetic labels and drugs. The moieties can be covalently linked polynucleotide recombinantly or by other means known in the art.

Polynucleotides can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments.

Polynucleotides can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service.

Polynucleotides comprising a desired sequence can be inserted into a suitable vector which in turn can be introduced into a suitable host cell for replication and amplification. Accordingly, a variety of vectors comprising one or more of the polynucleotides are provided. Also provided are selectable libraries of expression vectors comprising at least one vector encoding an antigen binding units disclosed herein.

Vectors generally comprises a transcriptional or translational control sequences required for expressing the antigen binding units. Suitable transcription or translational control sequences include but are not limited to replication origin, promoter, enhancer, repressor binding regions, transcription initiation sites, ribosome binding sites, translation initiation sites, and termination sites for transcription and translation.

The choice of promoters will largely depend on the host cells in which the vector is introduced. It is also possible, to utilize promoters normally associated with a desired light or heavy chain gene, provided that such control sequences are compatible with the host cell system. Cell-specific or tissue-specific promoters may also be used. A vast diversity of tissue specific promoters have been described and employed by artisans in the field. Exemplary promoters operative in selective animal cells include hepatocyte-specific promoters and cardiac muscle specific promoters. Depending on the choice of the recipient cell types, those skilled in the art will know of other suitable cell-specific or tissue-specific promoters applicable for the construction of the expression vectors.

Using known molecular cloning or gene engineering techniques, appropriate transcriptional control sequences, enhancers, terminators, or any other genetic element known in the art can integrated in operative relationship, optionally additionally with intact selectable fusion genes to be expressed. In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell.

The polynucleotides and vectors have several specific uses. They are useful, for example, in expression systems for the production of antigen binding units. Such polynucleotides are useful as primers to effect amplification of desired polynucleotides. Furthermore, polynucleotides are also useful in pharmaceutical compositions including vaccines, diagnostics, and drugs.

The host cells can be used, inter alia, as repositories of the subject polynucleotides, vectors, or as vehicles for producing and screening desired antigen binding units based on their antigen binding specificities.

Accordingly, provided is a method of identifying an antigen binding unit that is immunoreactive with a desired antigen. Such a method can involve the following steps: (a) preparing a genetically diverse library of antigen binding units, wherein the library comprises at least one subject antigen binding unit; (b) contacting the library of antigen binding units with the desired antigen; (c) detecting a specific binding between antigen binding units and the antigen, thereby identifying the antigen binding unit that is immunoreactive with the desired antigen.

The ability of an antigen binding unit to specifically bind to a desired antigen can be tested by a variety of procedures well established in the art. See Harlow and Lane (1988) Antibodies: A Laboratory Manual, Cold Spring Harbor Laboratory, New York; Gherardi et al. (1990) J. Immunol. Meth. 126:61-68. Typically, antigen binding units exhibiting desired binding specificities can be detected directly by immunoassays, for example, by reacting labeled antigen binding units with the antigens that are immobilized on a solid support or substrate. In general, the substrate to which the antigen is adhered is fabricated with material exhibiting a low level of non-specific binding during immunoassay. An example solid support is made from one or more of the following types of materials: plastic polymers, glass, cellulose, nitrocellulose, semi-conducting material, and metal. In some examples, the substrate is petri dish, chromatography beads, magnetic beads, and the like.

For such solid-phase assays, the unreacted antigen binding units are removed by washing. In a liquid-phase assay, however, the unreacted antigen binding units are removed by some other separation technique, such as filtration or chromatography. After binding the antigen to the labeled antigen binding units, the amount of bound label is determined. A variation of this technique is a competitive assay, in which the antigen is bound to saturation with an original binding molecule. When a population of the subject antigen binding unit is introduced to the complex, only those that exhibit higher binding affinity will be able to compete, and thus remain bound to the antigen.

Alternatively, specific binding to a given antigen can be assessed by cell sorting, which involves presenting the desired antigen on the cells to be sorted, then labeling the target cells with antigen binding units that are coupled to detectable agents, followed by separating the labeled cells from the unlabeled ones in a cell sorter. A sophisticated cell separation method is fluorescence-activated cell sorting (FACS). Cells traveling in single file in a fine stream are passed through a laser beam, and the fluorescence of each cell bound by the fluorescently labeled antigen binding unit is then measured.

Subsequent analysis of the eluted antigen binding units may involve protein sequencing for delineating the amino acid sequences of the light chains and heavy chains. Based on the deduced amino acid sequences, the cDNA encoding the antibody polypeptides can then be obtained by recombinant cloning methods including PCR, library screening, homology searches in existing nucleic acid databases, or any combination thereof. Commonly employed databases include but are not limited to GenBank, EMBL, DDBJ, PDB, SWISS-PROT, EST, STS, GSS, and HTGS.

When a library of antigen binding unit is displayed on phage or bacterial particles, selection is preferably performed using affinity chromatography. The method typically proceeds with binding a library of phage antigen binding units to an antigen coated plates, column matrices, cells or to biotinylated antigen in solution followed by capture. The phages or bacteria bound to the solid phase are washed and then eluted by soluble hapten, acid or alkali. Alternatively, increasing concentrations of antigen can be used to dissociate the antigen binding units from the affinity matrix. For certain antigen binding units with extremely high affinity or avidity to the antigen, efficient elution may require high pH or mild reducing solution as described in WO 92/01047.

The efficiency of selection is likely to depend on a combination of several factors, including the kinetics of dissociation during washing, and whether multiple antigen binding units on a single phage or bacterium can simultaneously bind to antigens on a solid support. For example, antibodies with fast dissociation kinetics (and weak binding affinities) can be retained by use of short washes, multivalent display and a high coating density of antigen at the solid support. Conversely, the selection of antigen binding units with slow dissociation kinetics (and good binding affinities) can be favored by use of long washes, monovalent phages, and a low coating density of antigen.

Where desired, the library of antigen binding units can be pre-selected against an unrelated antigen to counter-select the undesired antigen binding units. The library may also be pre-selected against a related antigen in order to isolate, for example, anti-idiotypic antigen binding units.

### Host Cells

In some embodiments, the present disclosure provides host cells expressing any one of the antigen binding units disclosed herein. A subject host cell typically comprises a nucleic acid encoding any one of the antigen binding units disclosed herein.

In one embodiment, host cells transfected with the polynucleotides, vectors, or a library of the vectors described above are provided. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the antigen binding units can be determined using any nucleic acid or protein assay known in the art. For example, the presence of transcribed mRNA of light chain CDRs or heavy chain CDRs, or the antigen binding unit can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of antigen binding unit polynucleotide.

Expression of the vector can also be determined by examining the antigen binding unit expressed. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunoflourescent assays, and SDS-PAGE.

### Preparation of Antigen-Binding Units

In some embodiments, methods of producing any antigen binding unit disclosed herein are provided. The method comprises culturing host cells expressing the antigen binding unit under conditions suitable for expressing the antigen binding unit and isolating the antigen binding unit expressed by the host cell.

The expressed antigen binding units can be isolated using a variety of protein purification techniques known in the art. Generally, the antigen binding unit is isolated from culture media as secreted polypeptides, although they can be recovered from host cell lysates or bacterial periplasm, when directly produced without signal peptides. If the antigen binding units are membrane-bound, they can be solubilized by suitable detergent solutions commonly employed by artisans in the field. The recovered antigen binding units may be further purified by salt precipitation (e.g., with ammonium sulfate), ion exchange chromatography (e.g. on a cationic or anionic exchange column run at neutral pH and eluted with step gradients of increasing ionic strength), gel filtration chromatography (including gel filtration HPLC), and chromatography on tag-affinity column, or on affinity resins such as protein A, protein G, hydroxyapatite, and anti-immunoglobulin.

In addition, derivatized immunoglobulins with added chemical linkers, detectable moieties such as fluorescent dyes, enzymes, substrates, chemiluminescent moieties, specific binding moieties such as streptavidin, avidin, or biotin, or drug conjugates can be utilized in the methods and compositions.

Additionally disclosed herein are antigen binding unites conjugated to a chemically functional moiety. Typically, the moiety is a label capable of producing a detectable signal. These conjugated antigen binding units are useful, for example, in detection systems such as quantitation of tumor burden, and imaging of metastatic foci and tumor imaging. Such labels are known in the art and include, but are not limited to, radioisotopes, enzymes, fluorescent compounds, chemiluminescent compounds, bioluminescent compounds substrate cofactors and inhibitors. See, for examples of patents teaching the use of such labels, U.S. Pat. Nos. 3,817,837; 3,850,752; 3,939,350; 3,996,345; 4,277,437; 4,275,149; and 4,366,241. The moieties can be covalently linked to antigen binding units, recombinantly linked, or conjugated to antigen binding units through a secondary reagent, such as a second antibody, protein A, or a biotin-avidin complex.

Other functional moieties include signal peptides, agents that enhance immunologic reactivity, agents that facilitate coupling to a solid support, vaccine carriers, bioresponse modifiers, paramagnetic labels and drugs. Signal peptides is a short amino acid sequence that directs a newly synthesized protein through a cellular membrane, usually the endoplasmic reticulum in eukaryotic cells, and either the inner membrane or both inner and outer membranes of bacteria. Signal peptides can be at the N-terminal portion of a polypeptide or the C-terminal portion of a polypeptide and can be removed enzymatically between biosynthesis and secretion of the polypeptide from the cell. Such a peptide can be incorporated into an antigen binding unit to allow secretion of the synthesized molecules.

Agents that enhance immunologic reactivity include, but are not limited to, bacterial superantigens. Agents that facilitate coupling to a solid support include, but are not limited to, biotin or avidin. Immunogen carriers include, but are not limited to, any physiologically acceptable buffers. Bioresponse modifiers include cytokines, particularly tumor necrosis factor (TNF), interleukin-2, interleukin-4, granulocyte macrophage colony stimulating factor and y-interferons.

Suitable drug moieties include antineoplastic agents. Non-limiting examples include radioisotopes, vinca alkaloids such as the vinblastine, vincristine and vindesine sulfates, adriamycin, bleomycin sulfate, carboplatin, cisplatin, cyclophosphamide, cytarabine, dacarbazine, dactinomycin, duanorubicin hydrochloride, doxorubicin hydrochloride, etoposide, fluorouracil, lomustine, mechlororethamine hydrochloride, melphalan, mercaptopurine, methotrexate, mitomycin, mitotane, pentostatin, pipobroman, procarbaze hydrochloride, streptozotocin, taxol, thioguanine, and uracil mustard.

Immunotoxins, including antigen binding units, can be produced by recombinant means. Production of various immunotoxins is well-known in the art, and methods can be found, for example, in “Monoclonal Antibody-toxin Conjugates: Aiming the Magic Bullet,” Thorpe et al. (1982) Monoclonal Antibodies in Clinical Medicine, Academic Press, pp. 168-190; Vitatta (1987) Science 238:1098-1104; and Winter and Milstein (1991) Nature 349:293-299. Suitable toxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

Chemically functional moieties can be made recombinantly for instance by creating a fusion gene encoding the antigen binding unit and the functional moiety. Alternatively, the antigen binding unit can be chemically bonded to the moiety by any of a variety of well-established chemical procedures. For example, when the moiety is a protein, the linkage can be by way of heterobifunctional cross linkers, e.g., SPDP, carbodiimide glutaraldehyde, or the like. The moieties can be covalently linked, or conjugated, through a secondary reagent, such as a second antibody, protein A, or a biotin-avidin complex. Paramagnetic moieties and the conjugation thereof to antibodies are well-known in the art. See, e.g., Miltenyi et al. (1990) Cytometry 11:231-238.

### Methods of Use and Treatment

CCR8-specific antigen binding units and pharmaceutical compositions comprising the same can find a wide variety of applications, including, but not limited to, treatment and diagnosis.

In one embodiment, pharmaceutical compositions comprising a pharmaceutically acceptable excipient and any of the antigen binding units disclosed herein are provided.

In another embodiment, methods of eradicating an immune cell comprising contacting a population of immune cells with an effective amount of the antigen binding unit described herein are provided.

In yet another embodiment, methods of eradicating an immune cell comprising contacting a population of immune cells with an effective amount of the pharmaceutical composition described herein are provided.

In embodiments, the immune cell is in a subject such as for example, a human patient in need of treatment for a disease caused by errant immune cells, and/or requiring removal of immune cells.

In embodiments, the immune cell is a regulatory T cell (Treg). The Treg may be present in the subject as a resident tissue Treg, for example in a tumor tissue as a subpopulation of resident tumor infiltrating lymphocytes (TILs).

In embodiments, Tregs migration, eliminates Tregs through antibody dependent cellular cytotoxicity (ADCC), or both.

In embodiments, methods of treating a cancer in a subject comprise administering to a subject in need thereof, an effective amount of the antigen binding unit described herein and optionally repeating the administration step for a period of time, for example, on a regular basis of once daily, once weekly, once monthly, for 1, 2, 3, 4, 5, 6 months, or until the subject is free of the cancer.

In embodiments, methods of treating a cancer in a subject comprise administering to a subject in need thereof, an effective amount of the pharmaceutical composition comprising a pharmaceutically acceptable excipient and any antigen binding unit disclosed herein. In embodiments, the administration step is repeated according to a dosage regimen that is effective to treat the cancer, as evidenced by the subject being free of the cancer.

In embodiments, the subject is a human patient in need of anti-cancer therapy. The cancer can be a hematological cancer or a solid tumor. Hematological cancers include, but are not limited to, leukemia, lymphoma (such as non-Hodgkin lymphoma) and myeloma. Solid tumors include, but are not limited to, a colorectal cancer (CRC), a spleen cancer, a breast cancer, a non-small-cell lung cancer (NSCLC), a pancreatic cancer, a melanoma, bile duct carcinoma, gallbladder carcinoma, thyroid carcinoma, sarcoma, a kidney cancer, a bladder cancer, uterine corpus cancer, an ovarian cancer, a lung cancer, a prostate cancer, a, head and neck cancer, a thymic carcinoma, a hepatocarcinoma, a testicular carcinoma, a urothelial cancer, an esophageal tumor and a gastric tumor. In most cases, the effective amount is determined empirically via testing methods well known in the art. In one aspect, the antigen binding unit is administered at a dosage from about 0.1 mg/kg to about 10 mg/kg body weight.

Treatment of cancer can be evidenced by reducing growth of cancer cells including, but is not limited to, reducing proliferation of cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with AML, etc. Whether a substance, or a specific amount of the substance, is effective in treating cancer can be assessed using any of a variety of known diagnostic assays for cancer, including, but not limited to biopsy, contrast radiographic studies, CAT scan, and detection of a tumor marker associated with cancer in the blood of the individual. The substance can be administered systemically or locally, usually systemically.

In embodiments, treatment of cancer can be evidenced by reduced tumor volume. Tumor volume can be determined using any known method in the field. For example, tumor volume can be determined by measuring the tumor using a caliper. In such cases, two dimensions of the tumor can be measured, and tumor volume can be determined using the formula V=0.5 a×b², where a and b are a first and second diameter. In some cases, the first diameter is the long diameter or the larger of the two diameters. In some cases, the second diameter is the short diameter or the smaller of the two diameters.

In embodiments, treatment of cancer can be evidenced by reduced tumor volume. In some cases, tumor volume is reduced by a percentage within the range of 1% to 100%. In some examples, tumor volume is reduced by about 1%, 5%, 10%, 15%, 20%, 25%, 30%, 35%, 40%, 45%, 50%, 55%, 60%, 65%, 70%, 75%, 80%, 85%, 90%, 95%, or 100%. In some examples, tumor volume is reduced by at least about 1%, 5%, 10%, 15%, 20%, 25%, 30%, 35%, 40%, 45%, 50%, 55%, 60%, 65%, 70%, 75%, 80%, 85%, 90%, 95%, or 100%.

In embodiments, comparison of the effect of a subject antigen binding unit compared to a reference antigen binding unit can be determined by calculating anti-tumor effectiveness. In such cases, tumor volume can be measure such as described above. Alternatively, a different parameter of tumor size or another appropriate characteristic of the tumor can be determined or measured. When working with quantifiable characteristics such as tumor volume, the anti-tumor effectiveness can be determined by using the formula: T/C, where T is the selected measurement (e.g., tumor volume) for the treatment group and C is the selected measurement (e.g., tumor volume) for the control group. Anti-tumor effectiveness can be determined over any desired period of time and can be determined using average value from any desired number of samples. Anti-tumor effectiveness can be expressed as a number or a percent. In some examples, anti-tumor effectiveness can be about 1%, 5%, 10%, 15%, 20%, 25%, 30%, 35%, 40%, 45%, 50%, 55%, 60%, 65%, 70%, 75%, 80%, 85%, 90%, 95%, or 100%. In some examples, anti-tumor effectiveness can be at most 1%, 5%, 10%, 15%, 20%, 25%, 30%, 35%, 40%, 45%, 50%, 55%, 60%, 65%, 70%, 75%, 80%, 85%, 90%, 95%, or 100%. In some examples, anti-tumor effectiveness can be at least 1%, 5%, 10%, 15%, 20%, 25%, 30%, 35%, 40%, 45%, 50%, 55%, 60%, 65%, 70%, 75%, 80%, 85%, 90%, 95%, or 100%.

Compositions, e.g., antigen binding units and pharmaceutical compositions, disclosed herein can be administered using any medically appropriate procedure, e.g., intravascular (intravenous, intra-arterial, intra-capillary) administration, injection into the lymph nodes, etc. Intravascular injection may be by intravenous or intraarterial injection. An effective amount of a composition to be given to a particular patient will depend on a variety of factors, several of which will be different from patient to patient and can be determined empirically. Dosage of the composition will depend on the determined treatment regime, route of administration, the nature of the therapeutics, sensitivity of the tumor to the therapeutics, etc. Utilizing LDso animal data, and other information available for an antigen binding unit disclosed herein, a clinician can determine the maximum safe dose for an individual, depending on the route of administration. For instance, an intravenously administered dose may be more than a locally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular composition.

Methods for combination therapies in which an agent known to modulate other pathways, or other components of the same pathway, or even overlapping sets of target enzymes are used in combination with a subject antigen binding unit or pharmaceutical composition comprising a subject antigen binding unit are contemplated. In an embodiment, such therapy includes, but is not limited to, the combination of one or more antigen binding units of the disclosure with chemotherapeutic agents, therapeutic antibodies, and radiation treatment, to provide a synergistic or additive therapeutic effect.

Many chemotherapeutics are presently known in the art and can be used in combination with a subject antigen binding unit. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.RTM.; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the antigen binding units or pharmaceutical composition of the present disclosure can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

This disclosure further relates to a method for using a subject antigen binding unit or a pharmaceutical composition provided herein, in combination with radiation therapy for inhibiting abnormal cell growth or treating the hyperproliferative disorder in the mammal. Techniques for administering radiation therapy are known in the art, and these techniques can be used in the combination therapy described herein.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, β-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

The antigen binding units or pharmaceutical compositions of the disclosure can be used in combination with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors, MMP-9 (matrix-metalloprotienase 9) inhibitors, and COX-11 (cyclooxygenase 11) inhibitors, can be used in conjunction with an antigen binding unit of the disclosure and pharmaceutical compositions described herein. Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (e.g., MAP-1, MMP-3, MMP-4, MMP-5, MMP-6, MMP- 7, MMP-8, MMP-10, MMP-ll, MMP-12, andMMP-13). Some specific examples of MMP inhibitors useful in the disclosure are AG-3340, RO 32-3555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In some embodiments, the antigen binding units and pharmaceutical compositions described herein are formulated or administered in conjunction with liquid or solid tissue barriers also known as lubricants. Examples of tissue barriers include, but are not limited to, polysaccharides, polyglycans, seprafilm, interceed and hyaluronic acid.

In some embodiments, medicaments which are administered in conjunction with the subject antigen binding units include any suitable drugs usefully delivered by inhalation for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines or pentamidine; antihistamines, e.g., methapyrilene; anti-inflammatories, e.g., beclomethasone, flunisolide, budesonide, tipredane, triamcinolone acetonide or fluticasone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics e.g., ipratropium, atropine or oxitropium; hormones, e.g., cortisone, hydrocortisone or prednisolone; xanthines e.g., aminophylline, choline theophyllinate, lysine theophyllinate or theophylline; and therapeutic proteins and peptides, e.g., insulin or glucagon. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

Other exemplary therapeutic agents useful for a combination therapy include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated herein include diuretics, vasopressin, agents affecting the renal conservation of water, rennin, angiotensin, agents useful in the treatment of myocardial ischemia, anti-hypertensive agents, angiotensin converting enzyme inhibitors, 0-adrenergic receptor antagonists, agents for the treatment of hypercholesterolemia, and agents for the treatment of dyslipidemia.

Other therapeutic agents contemplated include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, β-lactam antibiotics, an agent comprising an aminoglycoside, protein synthesis inhibitors, drugs used in the chemotherapy of tuberculosis, Mycobacterium avium complex disease, and leprosy, antifungal agents, antiviral agents including nonretroviral agents and antiretroviral agents.

Examples of therapeutic antibodies that can be combined with an antigen binding unit of the disclosure include but are not limited to anti-receptor tyrosine kinase antibodies (cetuximab, panitumumab, trastuzumab), anti CD20 antibodies (rituximab, tositumomab), immune checkpoint inhibitor (such as anti-PD-1, anti-PD-L1 antibody, and anti-CTLA-4 antibody, example: Nivolumab, Pembrolizumab, atezolizumab (Atezolizumab), avermab (Avelumab), durvalumab (Durvalumab), and ipilimumab), and other antibodies such as alemtuzumab, bevacizumab, and gemtuzumab. In some embodiments, the combination treatment of CCR8-specific antigen binding units as disclosed herein and anti-PD-1 induce significant and/or synergistic tumor growth inhibition and survival benefit as exemplified in Examples 27 and 28.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

For treating renal carcinoma, one may combine an antigen binding unit of the present disclosure with sorafenib and/or Avastin. For treating an endometrial disorder, one may combine an antigen binding unit of the present disclosure with doxorubincin, taxotere (taxol), and/or cisplatin (carboplatin). For treating ovarian cancer, one may combine an antigen binding unit of the present disclosure with cisplatin (carboplatin), taxotere, doxorubincin, topotecan, and/or tamoxifen. For treating breast cancer, one may combine an antigen binding unit of the present disclosure with taxotere (taxol), gemcitabine (capecitabine), tamoxifen, letrozole, tarceva, lapatinib, PD0325901, avastin, herceptin, OSI-906, and/or OSI-930. For treating lung cancer, one may combine an antigen binding unit of the present disclosure with taxotere (taxol), gemcitabine, cisplatin, pemetrexed, Tarceva, PD0325901, and/or avastin.

Further therapeutic agents that can be combined with an antigen binding unit of the disclosure are found in Goodman and Gilman's “The Pharmacological Basis of Therapeutics” Tenth Edition edited by Hardman, Limbird and Gilman or the Physician's Desk Reference, both of which are incorporated herein by reference in their entirety.

The antigen binding units described herein can be used in combination with the agents disclosed herein or other suitable agents, depending on the condition being treated. Hence, in some embodiments the one or more antigen binding units of the disclosure will be co-administered with other agents as described above. When used in combination therapy, the antigen binding units described herein are administered with the second agent simultaneously or separately. This administration in combination can include simultaneous administration of the two agents in the same dosage form, simultaneous administration in separate dosage forms, and separate administration. That is, an antigen binding unit described herein and any of the agents described above can be formulated together in the same dosage form and administered simultaneously. Alternatively, an antigen binding unit of the disclosure and any of the agents described above can be simultaneously administered, wherein both the agents are present in separate formulations. In another alternative, an antigen binding unit can be administered sequentially with any one or more of the agents described above, or vice versa. In some embodiments of the separate administration protocol, an antigen binding unit of the disclosure and any of the agents described above are administered a few minutes apart, or a few hours apart, or a few days apart.

Further illustration of the development and use of antigen binding units, polynucleotides, vectors and host cells are provided in the Example section below. The examples are provided as a guide to a practitioner of ordinary skill in the art and are not meant to be limiting in any way.

### EXAMPLES

The following examples are given for the purpose of illustrating various embodiments and are not meant to be limiting.

### Example 1

### Transient Transfection of Human CCR8 or Cynomolgus CCR8

Stable cell lines HEK293 and CHO-K1 overexpressing human CCR8, cynomolgus CCR8 or murine CCR8 were generated in Genomeditech Inc. and used for immunization and antibody screening purposes. The CCR8 expressing lymphoma cell line HuT78 (TIB-161) was obtained from ATCC.

Twenty-four hours prior to transfection, cells were cultured to a density of approximately 5-8×10⁶ cells/mL at a viability of at least 95%. On the day of transfection, cells were plated at a density of 2×10⁶ cells/ml in ExpiCHO-S Expression Medium (50 mL) in the shake flask. 40 pL plasmid DNA and 160 μL EXPIFECTAMINE CHO reagent were incubated at room temperature, and the solution was slowly transferred to the shake flask, while swirling the flask gently during addition. The cells were incubated on an orbital shaker at 37° C. in a C02 incubator.

433H monoclonal antibody (BD Biosciences, #644092), which specifically binds human CCR8, and 10A11-1 as described inW02020138489 were used as reference antibodies.

### Example 2

### Alignment of Human, Cynomolgus and Murine CCR8 Sequences

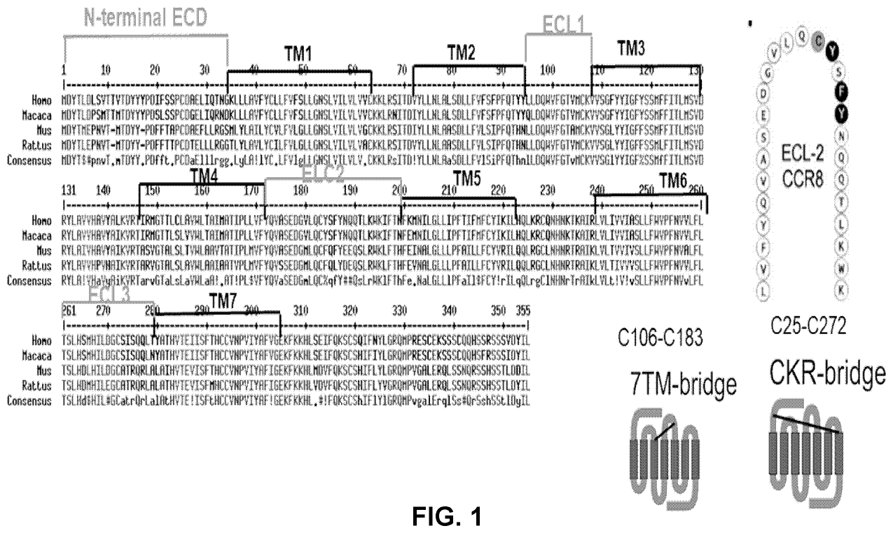

Human CCR8, cynomolgus and murine CCR8 sequences were retrieved from Uniprot and aligned (FIG. 1). While pairwise alignment of human and cynomolgus CCR8 yields a sequence identity of 94.37%, the identities were between mouse and human CCR8 (Percentage of identity=70.99%), and between mouse and cynomolgus CCR8 (Percentage of identity=71.55%), substantial differences were observed in the N-terminal extracellular domain (ECD) region and the C-terminal region. The sequence homology between the cynomolgus CCR8 ECD and the human CCR8 ECD is 68%, while sequence homology between the murine CCR8 ECD and the human CCR8 ECD is 52%. Nevertheless, the acidic and tyrosine sulfated ECD shows a negatively charged cluster which was successfully used to generate cross-reactive antibodies.

### Example 3

### Immunization and Hybridoma

Immunization of Balb/c and SJL mouse to generate antibody was conducted using the gene gun method. Gene gun cartridges for genetic immunization were prepared by mixing the human CCR8 plasmid with other immunoadjuvant plasmids including mouse GM-CSF and mouse GM-FLT3L (Tables 4-6). In order to induce antibodies cross-reactive to both human and cynomolgus CCR8, mice were immunized with human CCR8 plasmid 5 times and then immunized with cynomolgus CCR8 plasmid. Serum titer of the immunized mice was detected 7 days after every immunization by FACS using the human CCR8 overexpression cell line. The mice with the best titer were selected for the final boost with HEK293-cynomolgus CCR8 cell line 3 days before hybridoma fusion.

TABLE 4
The ratio of plasmid to make cartridge scheme

| Reagent name | Plasmid #1 | Plasmid #2 | Plasmid#3 | Ratio |
|---|---|---|---|---|
| Human-CCR8 | mouse | mouse GM-FLT3L | Human-CCR8 | 1:1:2 |
| cartridge | GM-CSF | | | |
| Cynomolgus-CCR8 | mouse | mouse GM-FLT3L | Cynomolgus- | 1:1:2 |
| cartridge | GM-CSF | | CCR8 | |

TABLE 5
Mouse immunization with human CCR8 plasmid scheme

| Animal | | | | Dosage | |
|---|---|---|---|---|---|
| group | Strain | Immunization date | Immunogen | (μg/animal) | Adjuvant |
| G1(7896- | Balb/c | D0, D14, D28, | plasmid | 4, 4, 4, 4, 4 | GM-CSF & |
| 7990) | | D42, D56 | human-CCR8 | | FLT3L |
| G2(7901- | SJL | D0, D14, D28, | plasmid | 4, 4, 4, 4, 4 | GM-CSF & |
| 7905) | | D42, D56 | human-CCR8 | | FLT3L |
| G3(7906- | Balb/c | D0, D14, D28, | plasmid | 4, 4, 4, 4, 4 | GM-CSF & |
| 7910) | | D42, D56 | human-CCR8 | | FLT3L |
| G4(7911- | SJL | D0, D14, D28, | plasmid | 4, 4, 4, 4, 4 | GM-CSF& |
| 7915) | | D42, D56 | human-CCR8 | | FLT3L |

TABLE 6
Mouse immunization with cynomolgus CCR8 plasmid scheme

| Animal | | Immunization | | Dosage | |
|---|---|---|---|---|---|
| group | Strain | Day | Immunogen | (μg/animal) | Adjuvant |
| G1(7896- | Balb/c | D70, D84 | plasmid | 4, 4 | GM- |
| 7990) | | | cynomolgus- | | CSF & |
| | | | CCR8 | | FLT3L |
| G3(7906- | Balb/c | D70, D84 | plasmid | 4, 4 | GM- |
| 7910) | | | cynomolgus- | | CSF & |
| | | | CCR8 | | FLT3L |

### Hybridoma Fusion and Subcloning

Mouse splenocytes isolated from high titer Balb/c mice and a myeloma fusion partner were fused with an electric field-based electrofusion using a Cyto Pulse large chamber cell fusion electroporator (BTX, ECM2001). Single cell suspensions of splenic lymphocytes from immunized mice were fused to a half number of sp2/0-Ag14 (ATCC CRL1581) non secreting mouse myeloma cells. Resulting cells were plated at 2.0×10⁴ cells/well in flat bottom 96well cell culture plate in 200 ul selective DMEM medium containing high glucose (GIBICO, cat.no:11995-065) and 20% FBS (GIBICO, cat.no:10091-148) and supplement with 50× HAT (GIBICO, cat.no: 21060-017). After 7 days of culture in CO₂ incubator, the medium containing HAT in the 96well cell culture plate was replaced with medium containing HT supplement (100×), liquid [GIBICO, cat.no: 11067030] and 10% FBS.

After 10 days, primary screening was performed by FACS. In this screening, 50p of supernatants were transferred from the fusion plates into 96well round bottom plates (Corning Incorporated, 3799), and mixed with the cynomolgus CCR8 overexpression cell lines. After 1 hour incubation at 4° C., the cells were washed twice with FACS buffer (PBS with 1.5% FBS) by centrifuging the plates. Then the secondary antibody (Goat ani-mouse IgG labeled AF647) was added and incubated with the cells at 4° C. for 30 minutes. After two additional wash steps, the cells were made into cell suspension and were read on a BD FACS Celesta reader.

Hybridoma cells from the positive wells, which had strong binding signal to CCR8 overexpression cells in FACS screening, were transferred to 24-well plates. After 3-5 days of culture, cell supernatants from individual wells were characterized by FACS and other functional assays.

The parental hybridoma cell lines that have been identified as positive antibodies were subcloned by limiting dilution. After 7 days of culture, the positive and monoclonal cell lines were screened out by FACS. The monoclonal antibodies were produced from the promising clones for further characterization. After ranking and validation by different assays, several hybridoma cells were selected for sequencing and further analysis.

### Example 4

### Screening of Hybridoma Clones by Facs Binding and Af647-Ccl 1 Binding Blocking Assays

Binding of hybridoma supernatant on CHO-K1-human CCR8 and ExpiCHO-S cynomolgus CCR8 cells. A total of 660 96-well plates were seeded and screened by FACS binding from the hybridoma derived from immunized animals. A total of 338 clones were identified as positive based on the geometric mean fluorescence intensity (MFI) fold (MFI fold=CCR8 positive cells MFI/CCR8 negative cells MF)>5.

To obtain antibodies cross-reactive to human and cynomolgus CCR8, hybridomas from mice immunized with human and cynomolgus CCR8 DNAs were screened by FACS. Briefly, 50 μL ExpiCHO-S cells transfected with human CCR8 or cynomolgus CCR8 (cell density: 2×10⁶ cells/mL, viability>90%) were incubated with equal volume hybridoma supernatant in 96 well plate (Corning) at 4° C. for 1 hour. After washing with FACS buffer (DPBS containing 2% FBS), the cells/antibody mixture was stained with secondary antibody (Alexa Flour® 647-conjugated rabbit anti-mouse IgG, Jackson ImmunoResearch). Finally, the mixture was washed and resuspended with FACS buffer, and subjected to FACS analysis on BD FACS Celesta. The raw data was analyzed with FlowJo software.13 hybridomas from immunized mice showed strong binding to CHO-K1- human CCR8 and CHO-K1- cynomolgus CCR8 cells (Table 7).

### Blocking Activities of Hybridoma Supernatants on CHO-K1-Human CCR8 Cells.

CHO-K1-human CCR8 cells were incubated with hybridoma supernatant and 4 nM AF647 labeled human CCL1 at 4° C. for 60 minutes. The cells were washed with FACS buffer (PBS buffer with 2% FBS) three times, and then AF647 signals were detected by BD FACS Celesta equipment. Data were analyzed using Flowjo V10 software. Binding inhibition rates were plotted against antibody concentrations. 13 hybridomas from immunized mice showed remarkable blocking activities on CHO-K1- human CCR8 cells (Table 7).

TABLE 7
Hybridoma clone supernatants bind on CHO-K1-human CCR8, ExpiCHO-S cynomolgus
CCR8 cells and block hCCL1 binding on HEK293-human CCR8 cells

| | | Binding Ratio (GFP + MFI)/ | | |
|---|---|---|---|---|
| | | (GFP − MFI) | | Inhibition Rate |
| | | CHO-K1- | CHO-K1- | (%) |
| | | human- | Cynomolgus- | HEK293- |
| Fusion | Subclone | CCR8 | CCR8 | Human-CCR8 |
| Ref | 433H | 158 | 7.90 | 100 |
| F1210 | 149F2C10 | 170 | 98.2 | 99.2 |
| F1210 | 153D3G2 | 215 | 127 | 99.1 |
| F1210 | 160D1E3 | 168 | 92.2 | 99.1 |
| F1210 | 164G10D6 | 198 | 102 | 99.4 |
| F0402 | 499B1A5 | 34.4 | 35.1 | 95.1 |
| F1217 | 191E12H8 | 345 | 91.4 | 84.8 |
| F1217 | 204A2G12 | 145 | 133 | 94.7 |
| F1217 | 206F1B2 | 341 | 105 | 79.3 |
| F1217 | 234G4C12 | 413 | 101 | 88.9 |
| F1217 | 258H7F3 | 103 | 72.3 | 97.9 |
| F1217 | 262C2G7 | 404 | 99.8 | 92.4 |
| F1217 | 273H2G6 | 120 | 112 | 90.2 |
| F1224 | 348E2D11 | 169 | 74.2 | 100 |

### Example 5

### Binding and Blocking Activities of Purified Murine Antibodies

The binding affinities of purified antibodies were determined on human CCR8 and cynomolgus CCR8 over-expressing cells. Briefly, CFSE (Life technology, Cat #: C34554) labeled CHO-K1-human CCR8 cells mixed with CHO-K1 cells, CFSE labeled HEK293-human CCR8 cells mixed with H1EK293 cells, or CFSE labeled H1EK293-cynomolgus CCR8 cells mixed with HTEK293 cells were used in the assay. The mix ratio was 1:1. A total of 5×10⁴ mixed cells for each well were seeded in 96-well plate and washed by FACS buffer (DPBS containing 10% FBS) once. Cells were incubated by series diluted purified hybridoma antibodies for 1 hour at 4° C. Antibodies were prepared with 3-fold serial dilution ranging from 200 nM to 0.003 nM in FACS buffer. After primary antibody incubation, cells were washed by FACS buffer for three times. Then, cells were stained with secondary antibody (Alexa Flu647-conj gated rabbit anti-mouse IgG, Jackson ImmunoResearch, #315606046) at 1:600 dilution in FACS buffer and incubated for 0.5 hour at 4° C. After 3 times washed, Alexa Fluor 647 signals of the stained cells were detected by BD FACS Celesta and MFI were determined. FlowJo software was used for analysis. Data was plotted as the logarithm of antibody concentration versus mean fluorescence signals. EC₅₀ values were calculated in GraphPad Prism 8 (GraphPad Software) using a log(agonist) vs. response-Variable slope (4 parameters) curve fit. Twelve purified murine antibodies including 149F2C10, 153D4G2, 160D1E3, 164G10D6, 204A2G12, 206F1B2, 258H7F3, 262C2G7, 234G9C12, 191E12H8, 273H2G6 and 348E2D11 showed a dose-dependent binding to CHO-K1-human CCR8 and HEK293-human CCR8 cells. These antibodies also cross reacted with cynomolgus CCR8 and showed a dose-dependent binding on HEK293-cynomolgus CCR8 cells. All of antibodies did not bind to CHO-K1 and HEK293 cells. The EC₅₀s of the selected antibodies on human CCR8 are less than 5 nM except those of 262C2G7 and 234G9C12. The EC₅₀s of the selected antibodies on cynomolgus CCR8 are less than 1 nM except that of 348E2D11 (Table 8).

TABLE 8
Binding affinities of purified murine antibodies on CHO-K1-human
CCR8, HEK293-human CCR8 and HEK293-Cynomolgus CCR8 cells.

| | | Binding | | Binding | | Binding | |
|---|---|---|---|---|---|---|---|
| | | on | | on | | on | |
| | | CHO-K1- | | HEK293- | | HEK293- | |
| | | human | | human | | Cynomolgus | |
| | | CCR8 | | CCR8 | | CCR8 | |
| Fusion | Subclone | EC₅₀ | Max | EC₅₀ | Max | EC₅₀ | Max |
| Fusion | Subclone | (nM) | MFI | (nM) | MFI | (nM) | MFI |
| Ref | 433H | 0.124 | 4919 | 0.0878 | 3887 | NA* | 2008 |
| Isotype | Mouse | NA* | 21 | NA* | 349 | NA* | 396 |
| | IgG2a | | | | | | |
| F1210 | 149F2C10 | 0.878 | 50704 | 0.346 | 11997 | 0.479 | 8830 |
| F1210 | 153D4G2 | 0.640 | 51361 | 0.240 | 11615 | 0.699 | 10660 |
| F1210 | 160D1E3 | 0.456 | 51489 | 0.278 | 11926 | 0.422 | 8937 |
| F1210 | 164G10D | 0.652 | 54906 | 0.277 | 11864 | 0.320 | 8288 |
| F1217 | 204A2G12 | 0.334 | 10228 | 0.142 | 3620 | 0.163 | 3200 |
| F1217 | 206F1B2 | 3.91 | 20890 | 3.33 | 8136 | 0.335 | 5342 |
| F1217 | 258H7F3 | 0.364 | 3666 | 0.0600 | 1036 | 0.115 | 675 |
| F1217 | 262C2G7 | 126 | 86445 | 0.139 | 7207 | 0.216 | 2943 |
| F1217 | 234G9C12 | 18.5 | 73893 | 1.42 | 9845 | 0.212 | 4136 |
| F1217 | 191E12H | 2.25 | 24004 | 1.50 | 10693 | 0.397 | 5187 |
| F1217 | 273H2G6 | 0.137 | 8486 | 0.149 | 4681 | 0.154 | 3501 |
| F1224 | 348E2D11 | 0.397 | 51451 | 0.111 | 11012 | NA* | 7575 |

*Not Applicable

Blocking curves were generated to rank the CCL1 blocking activities of hybridoma antibodies. Briefly, a total of 5×10⁴ CHO-K1-human CCR8 cells or HEK293-human CCR8 cells for each well were seeded in 96-well plate. Cells were incubated with serial diluted purified hybridoma antibodies and 4 nM AF647 labeled human CCL1 (Almac, #CAF-07) for 1 hour at 4° C.

Antibodies were prepared with 3-fold serial dilution ranging from 200 nM to 0.01 nM in FACS buffer. Then, cells were washed by FACS buffer for three times after incubation. Alexa Fluor 647 signals of the stained cells were detected by BD FACS Celesta and MFI were determined. FlowJo software was used for analysis. Data was plotted as the logarithm of antibody concentration versus mean fluorescence signals. IC₅₀ values were determined in GraphPad Prism 8 (GraphPad Software) using a log(agonist) vs. response-Variable slope (4 parameters) curve fit.

As shown in Table 9, 12 purified murine antibodies including 1491F2C0, 1531D4G2, 160D1E3, 164G10D6, 204A2G12, 206F1B2, 2581H71F3, 262C2G7, 234G9C12, 191E12H8, 273H2G6 and 348E2D11 could block CCL1 binding.

TABLE 9
Activities of purified murine antibodies in blocking human CCL1
binding on CHO-K1-human CCR8 and HEK293-human CCR8 cells.

| | | Blocking CCL1 | | Blocking CCL1 | |
|---|---|---|---|---|---|
| | | binding on | | binding on | |
| | | CHO-K1-human | | HEK293-human | |
| | | CCR8 | | CCR8 | |
| | | | Max | | Max |
| | | IC₅₀ | Inhibition | IC₅₀ | Inhibition |
| Fusion | Subclone | (nM) | (%) | (nM) | (%) |
| Ref | 433H | 0.171 | 98.7 | 0.335 | 98.8 |
| Isotype | Mouse IgG2a | NA | NA | NA | NA |
| F1210 | 149F2C10 | 0.917 | 98.0 | 0.733 | 96.4 |
| F1210 | 153D4G2 | 0.661 | 98.2 | 0.836 | 96.2 |
| F1210 | 160D1E3 | 0.728 | 98.0 | 0.891 | 100 |
| F1210 | 164G10D6 | 0.602 | 97.9 | 0.599 | 96.6 |
| F1217 | 204A2G12 | 2.73 | 95.0 | 1.64 | 87.6 |
| F1217 | 206F1B2 | 16.5 | 84.8 | NA | 77.2 |
| F1217 | 258H7F3 | 0.810 | 97.5 | 0.660 | 96.8 |
| F1217 | 262C2G7 | 1.57 | 95.6 | 0.632 | 86.1 |
| F1217 | 234G9C12 | 1.34 | 96.8 | 0.812 | 94.0 |
| F1217 | 191E12H8 | 13.3 | 89.7 | 0.934 | 85.6 |
| F1217 | 273H2G6 | 2.48 | 91.3 | 0.782 | 85.8 |
| F1224 | 348E2D11 | 0.231 | 95.0 | 0.368 | 100 |

### Example 6

### Purified Antibodies Inhibit CCL1 Induced β-Arrestin Recruitment

In response to a stimulus, the binding of a ligand-such as CCL1 for CCR8-GPCRs can activate G protein independent signaling, such as β-Arrestin recruitment. This can result in the internalization of the chemokine receptor. The β-arrestin assay kit was purchased from Discover X. In brief, CCR8 is fused in a frame with a small enzyme donor fragment ProLink (PK) and co-expressed in cells stably expressing a fusion protein of β-arrestin and the larger, N-terminal deletion mutant of β-galactosidase (called enzyme acceptor or EA). Activation of the CCR8 stimulates binding of β-arrestin to the PK-tagged CCR8 and forces complementation of the two enzyme fragments, resulting in the formation of an active β-galactosidase enzyme. This interaction leads to an increase in enzyme activity that can be measured using chemiluminescent Path Hunter Detection Reagents. Briefly, the cells were stimulated with CCL1 with CCL1 (CN-07, Almac) at EC₈₀ (4 nM) to activate β-Arrestin recruitment, and inventive antibodies or reference antibody 433H was added to evaluate their ability to block activated β-Arrestin recruitment. β-Arrestin recruitment was blocked by reference antibody 433H, while the purified antibodies 149F2C10, 153D4G2, 160D1E3, 164G10C6, 204A2G12 and 206F1B2 showed partial inhibition of β-Arrestin recruitment (Table 10).

TABLE 10
Activities of purified antibodies in inhibiting
CCL1 induced β-Arrestin recruitment

| | CHO-K1-human | |
|---|---|---|
| | CCR8 β-Arrestin | |
| | | Max |
| | IC₅₀ | Inhibition |
| Subclone | (nM) | (%) |
| 433H | 1.06 | 89.9 |
| Mouse IgG2a | NA | 3.80 |
| 149F2C10 | 0.61 | 32.0 |
| 153D4G2 | 6.57 | 33.8 |
| 160D1E3 | 1.04 | 41.3 |
| 164G10D6 | 0.460 | 42.0 |
| 204A2G12 | 1.70 | 30.9 |
| 206A2G12 | 1.55 | 16.5 |

### Example 7

### Binding and Blocking Activities of Ptm Sites Removed Antibodies

The sequences of antibodies produced by hybridoma technology were analyzed for post-translational modifications (PTMs), which could cause problems during the development of a therapeutic protein such as increased heterogeneity, reduced bioactivity, reduced stability, immunogenicity, fragmentation, and aggregation. The potential impact of PTMs depends on their location and in some cases on solvent exposure. The CDRs of all sequences were analyzed for asparagine deamination, aspartate isomerization, free cysteine thiol groups, N-glycosylation, oxidation, and fragmentation by potential hydrolysis sites.

For 149F2C10, an Asn²⁸-Gly²⁹ (NG) deamidation site existed in CDR1 region of light chain, which may cause stability issues. To reduce the risk of deamidation new variants were designed to remove NG site. Binding activities of NG site removal mutants were tested on cells along with parent clone as a control.

Binding affinities of NG site removed antibodies were determined on human CCR8 and cynomolgus CCR8 overexpression cells. Briefly, CFSE labeled HEK293-human CCR8 cells or CFSE labeled HEK293-cynomolgus CCR8 cells were mixed with HEK293 cells. The mixed ratio was 1:1. A total of 5×10⁴ mixed cells for each well were seeded in 96-well plates and washed by FACS buffer (DPBS containing 1% FBS) once. Cells were incubated by serial diluted purified hybridoma antibodies for 1 hour at 4° C. Antibodies were prepared with 3-fold serial dilution ranging from 100 nM to 0.0017 nM or from 25.3165 nM to 0.0004 nM in FACS buffer. 433H was set as a positive control. After primary antibody incubation, cells were washed by FACS buffer for three times. Then, cells were stained with secondary antibody (Alexa Flu647-conj gated rabbit anti-mouse IgG, Jackson ImmunoResearch, #315606046) at 1:600 dilution in FACS buffer, incubated for 0.5 hour at 4° C. Alexa Fluor 647 signals of the stained cells were detected by BD FACS Celesta and the MFI were determined. FlowJo software was used for analysis. Data was plotted as the logarithm of antibody concentration versus mean fluorescence signals. EC₅₀ values were calculated in GraphPad Prism 8 (GraphPad Software) using a log (agonist) vs. response-Variable slope (4 parameters) curve fit.

Blocking curves were generated to rank the blocking activities of PTM sites removed antibodies. Briefly, a total of 5×10⁴ CHO-K1-human CCR8 cells for each well were seeded in 96-well plate. Cells were incubated by series diluted purified hybridoma antibodies and 4 nM AF647 labeled human CCL1 (Almac, #CAF-07) for 1 hour at 4° C. Antibodies were prepared with 3-fold serial dilution ranging from 100 nM to 0.0051 nM in FACS buffer. Then, cells were washed by FACS buffer for three times after incubation. Alexa Fluor 647 signals of the stained cells were detected by BD FACS Celesta and MFI were determined. FlowJo software was used for analysis. Data was plotted as the logarithm of antibody concentration versus mean fluorescence signals. IC₅₀ values were determined in GraphPad Prism 8 (GraphPad Software) using a log(agonist) vs. response-Variable slope (4 parameters) curve fit.

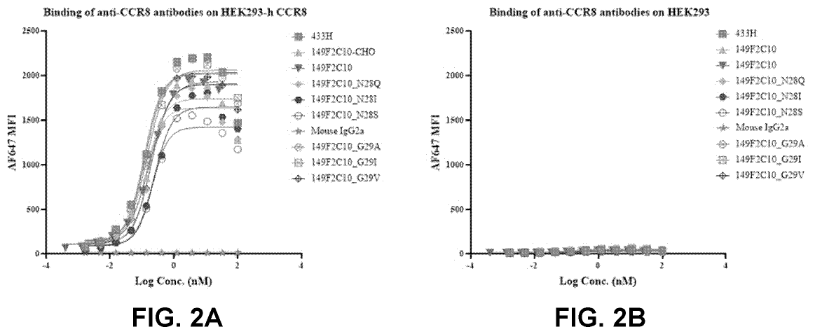

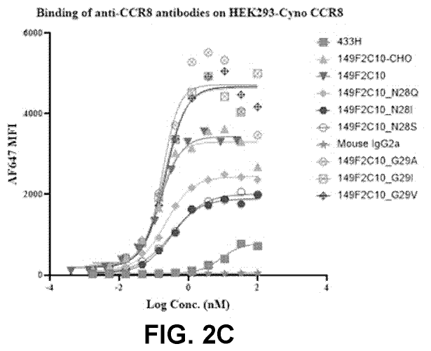

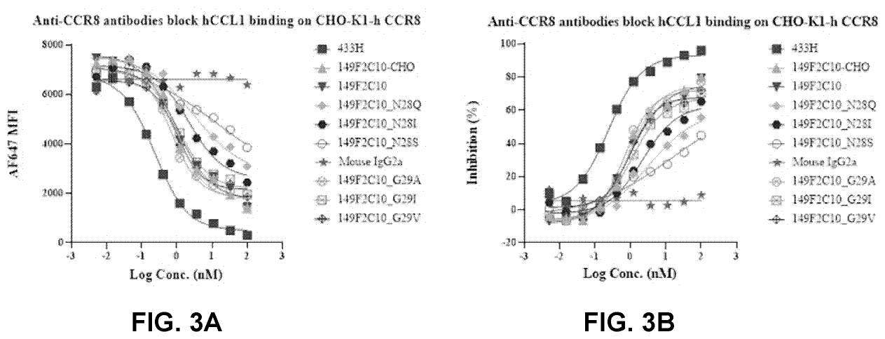

As shown in FIG. 2 and Table 11, 149F2C10_G29A showed the best binding activity on HEK293-human CCR8 and HEK293-Cynomolgus CCR8 among the PTM site removed antibodies and the EC₅₀s were 0.129 nM and 0.173 nM, respectively. None of the antibodies showed non-specific binding on HEK293 cells. As shown in FIG. 3 and Table 11, 149F2C10_G29A showed the best blocking activity on CHO-K1-human CCR8 among the PTM site removed antibodies and the IC₅₀ was 0.725 nM.

TABLE 11
Binding and blocking activities of PTM sites removed antibodies on HEK293-
human CCR8, HEK293-Cynomolgus CCR8 and CHO-K1-human CCR8 cells.

| | | | | | Blocking | |
|---|---|---|---|---|---|---|
| | Binding | | Binding | | on | |
| | on | | on | | CHO-K1- | |
| | HEK293- | | HEK293- | | human | |
| | human | | Cynomolgus | | CCR8 | |
| | CCR8 | | CCR8 | | | Max |
| | EC₅₀ | Max | EC₅₀ | Max | IC₅₀ | Inhibition |
| Antibody | (nM) | MFI | (nM) | MFI | (nM) | (%) |
| 433H | 0.117 | 2022 | 9.08 | 802 | 0.243 | 96.0 |
| Mouse | NA¹ | 11.0 | NA¹ | 47.0 | NA¹ | 12.3 |
| IgG2a | | | | | | |
| 149F2C10-CHO | 0.142 | 1743 | 0.131 | 3309 | 0.934 | 80.1 |
| 149F2C10 | 0.173 | 1933 | 0.145 | 3439 | 0.970 | 79.4 |
| 149F2C10_N28Q | 0.161 | 1638 | 0.209 | 2450 | 3.07 | 55.7 |
| 149F2C10_N28I | 0.244 | 1648 | 0.314 | 1893 | 2.25 | 65.3 |
| 149F2C10_N28S | 0.210 | 1423 | 0.333 | 2022 | 15.0 | 44.9 |
| 149F2C10_G29A | 0.129 | 2035 | 0.173 | 4725 | 0.725 | 77.5 |
| 149F2C10_G29I | 0.146 | 2062 | 0.190 | 4645 | 1.09 | 70.7 |
| 149F2C10_G29V | 0.194 | 1903 | 0.211 | 4675 | 1.04 | 72.0 |

¹Not Applicable

### Example 8

### Humanization of Anti-CCR8 Antibodies

Humanization of selected candidates 149F2C10_G29A (abbreviated as “149”) and 348E2D11 (abbreviated as “348”) was performed by grafting CDRs residues from mouse antibody onto a human germline framework. First, the sequences of the VH and VL region of selected candidates were compared with human germline sequences, and the best-fit germline acceptors were selected based on homology, canonical structure and physical properties. Subsequently, structure models of candidates were generated using homology modelling. The CDR regions in both heavy and light chains of candidate antibodies were fixed, and the murine frameworks were replaced with selected human germline frameworks. Different residues between mouse and human frameworks that potentially influence CDR conformation or VH/VL interface were subjected to back mutation. DNA fragments encoding the designed humanized variants were synthesized and subcloned into IgG expression vectors. DNA sequences were confirmed by sequencing. Different combinations of humanized heavy and light chains were co-transfected into CHO-K1 cells for expression. Finally, nine humanized variants including Hu149-1, Hu149-2, Hu149-3, Hu149-4, Hu149-5, Hu149-6, Hu149-7, Hu149-8 and Hu149-9 were derived from 149F2C10_G29A. Nine humanized variants including Hu348-1, Hu348-2, Hu348-3, Hu348-4, Hu348-5, Hu348-6, Hu348-7, Hu348-8 and Hu348-9 were derived from 348E2D 11. The humanized antibodies were compared with parental antibody for antigen binding affinity by FACS using cells expressing the target antigen.

### Example 9

### Binding and Blocking Activities of Humanized 149

A total of 5×10⁴ cells for each well were seeded in 96-well plates and washed with FACS buffer (DPBD containing 1.5% FBS) for once. Antibodies were prepared with 3-fold serial dilution ranging from 100 nM to 0.0017 nM in FACS buffer. Cells were incubated with 50 μL of diluted antibodies at 4° C. for 1 hour. Control group comprised cells incubated with human IgG1, 433H or mouse IgG2a. After primary antibody incubation, cells were washed for 3 times by FACS buffer. Then, cells were stained by Alexa Flu647 labeled anti-human IgG secondary antibody (Jackson ImmunoResearch, #109606170) or Alexa Flu647 labeled anti-mouse IgG secondary antibody (Jackson ImmunoResearch, #115605072) at 1:600 dilution in FACS buffer for 0.5 hour at 4° C. After washed for 3 times, Flow cytometry was performed to measure the binding.

The binding affinities of humanized 149 antibodies were tested using HEK293-human CCR8 and HEK293-Cynomolgus CCR8 cells. The blocking activities of humanized 149 antibodies were tested using CHO-K1-human CCR8 cells. As shown in Tables 12-13, Hu149-4 and Hu149-9 showed best binding activities among the humanized antibodies on HEK293-human CCR8 and HEK293-Cynomolgus CCR8 cells in two separate experiment settings. As shown in Tables 12-13, Hu149-4 and Hu149-9 showed most potent blocking activities among the humanized antibodies on CHO-K1-human CCR8 cells, the IC₅₀ s were 0.137 nM and 0.119 nM, respectively.

TABLE 12
Binding and blocking activities of humanized
149 antibodies on HEK293-human CCR8, HEK293-
Cynomolgus CCR8 and CHO-K1-human CCR8 cells.

| | | | | | CCL1 | |
|---|---|---|---|---|---|---|
| | Binding | | Binding | | binding | |
| | on | | on | | blocking | |
| | HEK293- | | HEK293- | | on CHO-K1- | |
| | human | | Cynomolgus | | human CCR8 | |
| | CCR8 | | CCR8 | | | Max |
| | EC₅₀ | Max | EC₅₀ | Max | IC₅₀ | Inhibition |
| Antibody | (nM) | MFI | (nM) | MFI | (nM) | (%) |
| 433H | 0.119 | 2807 | NA¹ | 1236 | 0.198 | 99.6 |
| CM149 | 0.142 | 7099 | 0.235 | 18147 | 0.681 | 97.8 |
| Hu149-1 | 0.534 | 7611 | 0.578 | 20912 | 0.936 | 99.2 |
| Hu149-2 | 3.48 | 1118 | 146 | 1546 | 12.5 | 39.5 |
| Hu149-3 | 0.193 | 7002 | 0.205 | 19914 | 0.478 | 99.2 |
| Hu149-4 | 0.0518 | 7471 | 0.081 | 26490 | 0.137 | 99.5 |
| Hu149-5 | 15.0 | 2466 | NA¹ | 414 | 49.6 | 57.3 |
| Hu149-6 | NA¹ | 138 | NA¹ | 443 | 2.37 | 1.00 |
| Hu149-7 | 6.42 | 3112 | NA¹ | 488 | 37.8 | 59.0 |
| Hu149-8 | 0.240 | 3829 | NA¹ | 2419 | 1.24 | 86.5 |
| Human | NA¹ | 180 | NA¹ | 331 | NA¹ | −13.0 |
| IgG1 | | | | | | |
| Mouse | NA¹ | 18 | NA¹ | 40 | NA¹ | −13.9 |
| IgG2a | | | | | | |

¹Not Applicable

TABLE 13
Binding and blocking activities of Hu149-9
antibody on HEK293-human CCR8, HEK293-Cynomolgus
CCR8 and CHO-K1-human CCR8 cells.

| | | | | | Blocking | |
|---|---|---|---|---|---|---|
| | Binding | | Binding | | on | |
| | on | | on | | CHO-K1- | |
| | HEK293- | | HEK293- | | human | |
| | human | | Cynomolgus | | CCR8 | |
| | CCR8 | | CCR8 | | | Max |
| | EC₅₀ | Max | EC₅₀ | Max | IC₅₀ | Inhibition |
| Antibody | (nM) | MFI | (nM) | MFI | (nM) | (%) |
| 433H | 0.215 | 5944 | NA¹ | 2340 | 0.193 | 99.1 |
| CM149 | 0.180 | 7113 | 0.263 | 22340 | 0.708 | 97.1 |
| Hu149-9 | 0.072 | 7532 | 0.120 | 30994 | 0.119 | 99.1 |
| Human | NA¹ | 222 | NA¹ | 120 | NA¹ | 27.4 |
| IgG1 | | | | | | |
| Mouse | NA¹ | 261 | NA¹ | 158 | NA¹ | 28.7 |
| IgG2a | | | | | | |

¹Not Applicable

### Example 10

### Binding and Blocking Activities of Humanized 348

The binding affinities of humanized 348 antibodies were tested using HEK293-human CCR8 and ExpiCHO-S-Cynomolgus CCR8 cells. The blocking activities of humanized 348 antibodies were tested using CHO-K1-human CCR8 cells. As shown in Table 14, Hu348-1, Hu348-2, Hu348-3, Hu348-4, Hu348-8, Hu348-9, parental antibody CM348 and 433H showed comparable binding affinities on HEK293-human CCR8 and the EC₅₀s were 0.122 nM, 0.119 nM, 0.108 nM, 0.109 nM, 0.144 nM, 0.182 nM, 0.197 nM and 0.091 nM, respectively. Intriguingly, Hu348-4 showed significantly improved best binding on ExpiCHO-S cynomolgus CCR8 cells as compared to the parental antibody CM348 with an EC₅₀ value of 0.087 nM (Table 14). Hu348-4 and the parental antibody CM348 showed similar CCL1 binding blocking activities, the IC₅₀ s were 0.173 nM and 0.151 nM, respectively (Table 14).

TABLE 14
Binding and blocking activities of humanized
348 antibodies on HEK293-human CCR8, ExpiCHO-
S-Cynomolgus CCR8 and CHO-K1-human CCR8 cells.

| | | | | | Blocking | |
|---|---|---|---|---|---|---|
| | Binding | | Binding | | on | |
| | on | | on | | CHO-K1- | |
| | HEK293- | | ExpiCHO-S | | human | |
| | human | | Cynomolgus | | CCR8 | |
| | CCR8 | | CCR8 | | | Max |
| | EC₅₀ | Max | EC₅₀ | Max | | Inhibition |
| Antibody | (nM) | MFI | (nM) | MFI | IC₅₀ (nM) | (%) |
| 433H | 0.197 | 2779 | NA¹ | 27 | 0.143 | 99.8 |
| CM348 | 0.091 | 5053 | 65.3 | 238 | 0.170 | 96.4 |
| Hu348-1 | 0.122 | 5110 | NA¹ | 336 | 0.230 | 94.1 |
| Hu348-2 | 0.119 | 4742 | NA¹ | 296 | 0.216 | 92.4 |
| Hu348-3 | 0.108 | 4092 | NA¹ | 350 | 0.362 | 83.1 |
| Hu348-4 | 0.109 | 5123 | 0.087 | 534 | 0.151 | 94.6 |
| Hu348-5 | NA¹ | 163 | NA¹ | 6 | NA¹ | 1.39 |
| Hu348-6 | 0.978 | 1980 | 31.1 | 82 | 1.84 | 78.3 |
| Hu348-7 | NA¹ | 756 | NA¹ | 174 | NA¹ | 25.6 |
| Hu348-8 | 0.144 | 4732 | NA¹ | 267 | 0.358 | 88.4 |
| Hu348-9 | 0.182 | 4496 | NA¹ | 148 | 0.333 | 88.2 |
| Human | NA¹ | 167 | NA¹ | 13 | NA¹ | −4.1 |
| IgG1 | | | | | | |
| Mouse | NA¹ | 49 | NA¹ | 5 | NA¹ | 2.1 |
| IgG2a | | | | | | |

¹Not Applicable

### Example 11

### Selected Humanized Antibodies do not Bind to Human CCR1 or CCR4

CCR1 is a receptor for a C—C type chemokine. It binds to MIP-1-alpha, MIP-1-delta, RANTES, and MCP-3 and, less efficiently, to MIP-1-beta or MCP-1 and subsequently transduces a signal by increasing the intracellular calcium ions level. CCR1 is responsible for stem cell proliferation.

CCR4 is a high affinity receptor for the C—C type chemokines CCL 17/TARC, CCL22/MDC and CKLF isoform 1/CKLF 1. The activity of this receptor is mediated by G(i) proteins which activate a phosphatidylinositol-calcium second messenger system. It can function as a chemoattractant homing receptor on circulating memory lymphocytes and as a coreceptor for some primary HIV-2 isolates. In the CNS, CCR4 could mediate hippocampal-neuron survival.

Human CCR8, CCR1 and CCR4 sequences were retrieved from Uniprot. The sequences were aligned for calculation of sequence identity. While pairwise alignment of CCR1 and CCR8 yields a sequence identity of 39.3300, percentages are similar between CCR4 and CCR8 (Percentage of identity=43.13%). The sequence homology between CCR1 ECD and CCR8 ECD was 22.9%, while sequence homology between CCR4 ECD and CCR8 ECD was 31.4%.

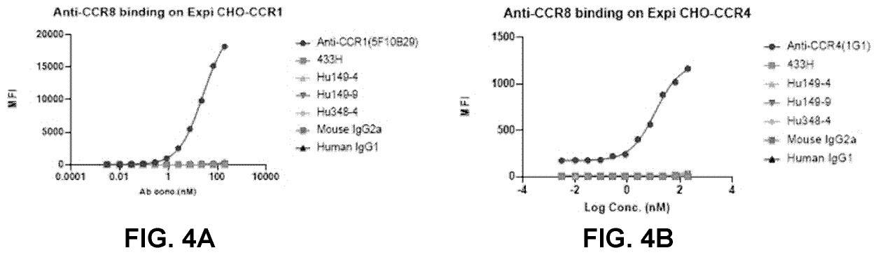

ExpiCHO-S cells were transiently transfected with human CCR1 or human CCR4. A total of 5×10⁴ cells for each well were seeded in 96-well plates and washed with FACS buffer (DPBS containing 1.5% FBS) for once. Antibodies were prepared with 3-fold serial dilution ranging from 200 nM to 0.00338 nM in FACS buffer. Cells were incubated with 50 μL of diluted antibodies at 4° C. for 1 hour. Control group comprised cells incubated with human IgG1, 433H or mouse IgG2a. After primary antibody incubation, cells were washed for 3 times by FACS buffer. Then, cells were stained by Alexa Flu647 labeled anti-human IgG secondary antibody (Jackson ImmunoResearch, #109606170) or Alexa Flu647 labeled anti-mouse IgG secondary antibody (Jackson ImmunoResearch, #115605072) at 1:600 dilution in FACS buffer for 0.5 hour at 4° C. Flow cytometry was performed to measure the binding. As shown in FIG. 4 and Table 15, anti-CCR1 antibody (5F10B29) showed dose-dependent binding to ExpiCHO-CCR1 cells and the EC₅₀ was 24.7 nM, while all the selected humanized antibodies showed no or minimal binding on Expi CHO-CCR1 cells; anti-CCR4 antibody (1G1) showed dose-dependent binding to ExpiCHO-CCR4 cells and the EC₅₀ was 11.7 nM, while all the selected humanized antibodies shown no or minimal binding on Expi CHO-CCR4 cells.

TABLE 15
Binding affinities of selected humanized
antibodies on ExpiCHO-S-human CCR1
and ExpiCHO-S-human CCR4 cells.

| | ExpiCHO-S | | ExpiCHO-S | |
|---|---|---|---|---|
| | CCR1 | | CCR4 | |
| | EC₅₀ | Max | EC₅₀ | Max |
| Antibody | (nM) | MFI | (nM) | MFI |
| 5F10B29 | 24.7 | 18178 | ND² | ND² |
| 1G1 | ND² | ND² | 11.7 | 1164 |
| 433H | NA¹ | 133 | NA¹ | 6.10 |
| Hu149-4 | NA¹ | 536 | NA¹ | 44.0 |
| Hu149-9 | NA¹ | 226 | NA¹ | 32.8 |
| Hu348-4 | NA¹ | 22.8 | NA¹ | 8.90 |
| Hu IgG1 | NA¹ | 20.3 | NA¹ | 7.00 |
| Mouse IgG2a | NA¹ | 267 | NA¹ | 6.60 |

¹Not Applicable,
²Not Done

### Example 12

### Antibody Dependent Cellular Cytotoxicity (ADCC) Function of Selected Humanized Antibodies

Human PBMC based ADCC activities of humanized antibodies were tested on CHO-K1 human CCR8 cells. Cryopreserved PBMCs (AllCells) of a healthy subject were thawed one day before the assay and cultured overnight in RPMI160 medium with 10% FBS and 200 IU JIL-2 (R&D, Cat #: 202-IL) in a C02 incubator. The target cells were labeled by CFSE (Life technology, Cat #: C34554) at the final concentration of 2.5 μM for 15 minutes. After staining, cell concentration was adjusted to 6×10⁴ cells/mL and mixed with 2-times volume of PBMCs which were adjusted to 1×10⁶ cells/mL (effector cell/target cell ratio was 40:1). Then, 150 μL of mixed target and effector cell suspension and 50 μL of series diluted antibodies were mixed in each well. Duplicate wells were prepared for each concentration of antibody. Target cells alone were used as control group. After incubation at 37° C., 5% C02 for 5 hours, 1 μg/mL PI (Invitrogen, Cat #: 51-66211E) was added and analyzed by flow cytometry (BD FACS Celesta). Specific cytotoxicity was calculated by the formula: specific cytotoxicity=% PI positive cell with antibody- % PI positive cell without antibody.

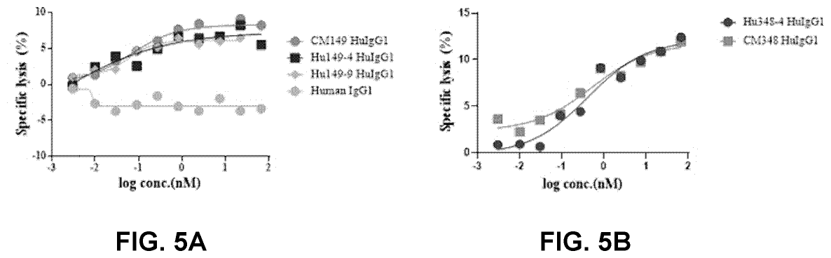

Hu149-4 HuIgG1, Hu149-9 HuIgG1 and the parental antibody CM149 HuIgG1 showed similar ADCC activities on CHO-K1 cells overexpressing human CCR8. Humanized Hu348-4 HuIgG1 and the parental antibody CM348 HuIgG1 showed similar ADCC activities on CHO-K1 cells overexpressing human CCR8 (FIG. 5, Table 16).

TABLE 16
ADCC activities of humanized antibodies
on CHO-K1-human CCR8 cell line

| | | Max |
|---|---|---|
| | | specific |
| | EC₅₀ | lysis |
| Antibody | (nM) | (%) |
| CM149 HuIgG1 | 0.0670 | 10.3 |
| Hu149-4 HuIgG1 | 0.00200 | 8.76 |
| Hu149-9 HuIgG1 | 0.0600 | 7.56 |
| Human IgG1 | NA | 0.860 |
| CM348 HuIgG1 | 0.410 | 13.1 |
| Hu348-4 HuIgG1 | 0.517 | 13.5 |

¹Not Applicable

### Example 13

### Selected Humanized Antibodies Inhibit Ccl 1 Induced B-Arrestin Recruitment

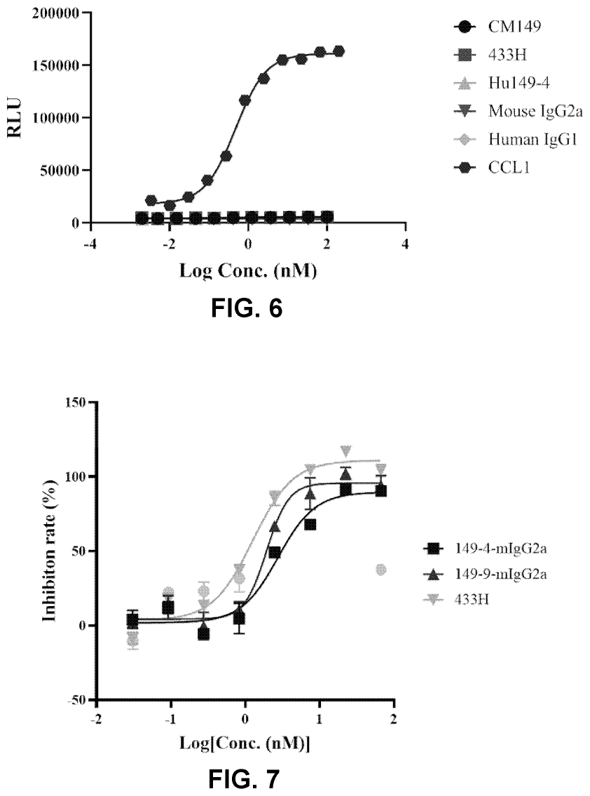

In a first experiment, CHO-K1 cells co-expressing human CCR8 tagged with ProLink (PK) and Enzyme Acceptor (EA) were incubated with antibody or CCL 1 (CN-07, Almac) for 90 minutes before adding detection reagent (93-0001, DiscoverX). CCL1 induced β-arrestin recruitment in a dose dependent manner and the EC₅₀ was 0.288 nM. The reference antibody 433H, CM149 or Hu149-4 did not induce β-arrestin recruitment (FIG. 6).

CHO-K1 CCR8 β-arrestin cells were stimulated with CCL1 (CN-07, Almac) at EC₈₀ (4 nM) to activate β-Arrestin recruitment and selected humanized antibodies or reference antibodies were added to evaluate their ability to block activated β-Arrestin recruitment. Because subtype of the reference antibody 433H was mouse IgG2a, the subtype of all test antibodies was changed to mouse IgG2a. As shown in Table 17, Hu149-9 and Hu10A11-1 showed similar inhibitory activities on 0-Arrestin recruitment, the IC₅₀s were 8.64 nM and 17.5 nM, respectively. 433H showed better inhibitory activities of β-Arrestin recruitment than Hu149-9, the IC₅₀s were 0.23 nM and 8.64 nM, respectively.

TABLE 17
Selected humanized antibodies inhibit
CCL1 induced β-Arrestin recruitment in
CHO-K1-human CCR8 β-Arrestin cells

| | CHO-K1-human | |
|---|---|---|
| | CCR8 β-Arrestin | |
| | recruitment | |
| | | Max |
| | IC₅₀ | inhibition |
| Antibody | (nM) | (%) |
| 433H | 0.230 | 92.6 |
| Hu149-9 mIgG2a | 8.64 | 72.5 |
| Hu348-4 mIgG2a | 860 | 14.3 |
| Hu10A11 mIgG2a | 48.2 | 64.7 |
| Hu10A11-1 mIgG2a | 17.5 | 74.7 |
| Hu19D7 mIgG2a | 1.28 | 91.3 |
| Mouse IgG2a | NA¹ | 26.0 |

¹Not Applicable

### Example 14

### Humanized Antibodies Inhibit Ccl 1 Induced Cell Migration

Activities of humanized anti-CCR8 antibodies in blocking CCL1 induced cell migration were evaluated. CHO-K1-human CCR8 cells with or without anti-CCR8 antibodies were added into upper chamber of transwell-96 permeable support. Chemoattractant, CCL1 was added into lower chamber. Cells passed through the membrane from the top and stick on the back side of the membrane. Cells were dissociated from the membrane using Trypsin and quantitated by Cell titer Glo. As shown in FIG. 7, Hu149-4-mIgG2a, Hu149-9-mIgG2a and the reference antibody 433H were tested in the migration assay. Hu149-4-mIgG2a, Hu149-9-mIgG2a and 433H blocked CCL1 induced cell migration. The IC₅₀s were 2.95 nM, 1.92 nM and 1.25 nM, respectively (FIG. 7, Table 18).

$$Inhibition rate ( \% ) = ( 1 - \frac{chem o taxis index ( samples ) - 1}{c h e m o taxis ( without antibody ) - 1} ) \times 100 \\ Chemotaxis index = \frac{CTG va1ue ( samples )}{CTG va1ue ( without CCL 1 )}$$

TABLE 18
Activities of anti-CCR8 antibodies in
blocking CCL1 induced cell migration

| | | Max |
|---|---|---|
| | IC₅₀ | inhibition |
| Antibody | (nM) | (%) |
| Hu149-4-mIgG2a | 2.95 | 91.5 |
| Hu149-9-mIgG2a | 1.92 | 102 |
| 433H | 1.25 | 117 |

### Example 15

### Affinity Maturation of Exemplary Anti-CCR8 Antibodies Hu149-9

In order to improve the binding affinity of humanized variant 149-9 against human CCR8, phage display technology was utilized for affinity maturation. Parental antibody sequence was used as templates, assembled as VH-VL format with (G4S)₃ linker between, and cloned into pComb3XSS phagemid vector.

The scFv-containing phagemid vector was transformed into TG1 and antigen binding of phage-displayed 149-9 scFv was validated by FACS. Two CDR mutagenesis libraries target CDRH3 and CDRL3 were constructed separately by soft mutagenesis guided primer design and overlap PCR. CDR positions were defined by Kabat numbering system. Amino acid residues within the CDR3 loops were randomized with NNK codes. Mutation rate at each position was kept at ˜50% to maximally retain original binding epitope. During affinity-driven cell-based panning, harsh and moderate washing conditions were incorporated to reduce non-specific binders and wild type occupation.

### Example 16

### Screening of Affinity Matured Variants by Facs Binding

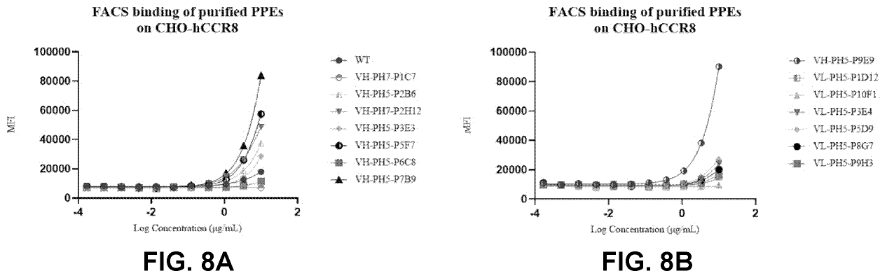

50˜100 random clones were sequenced for each library, and sequence alignment with the wild-type sequence was used to evaluate the quality of the mutation libraries including mutation positions and mutation rate. Up to 5500 single clones were selected for periplasmic extract (PPE) preparation and FACS binding screening (FIGS. 8A-B). Clones with high signal/background ratio were subjected to sequencing and 14 scFvs were purified and then confirmed by FACS binding followed by human IgG1 conversion including combination of the beneficial mutations from heavy and light chains. After transient expression in REK293 cells, the IgGs in the culture supernatant were subjected to one-step protein A purification. The purified IgGs were further characterized upon purity by SDS-PAGE, aggregation by SEC-HPLC, and concentration by UV280. Antigen-binding were validated by FACS (Tables 19-20). Given that ADCC effects could greatly contribute to Treg depletion, Fc mutations including S239D/I332E (US2005054832A1) and L235V/F243L/R292P/Y300L/P396L (AU2004204494B2) were introduced.

TABLE 19
FACS binding data of purified scFvs
on CHO-K1-human CCR8 cells

| | GeoMean AF647 |
|---|---|
| | (RL1-H) of |
| | CHO-K1-human |
| | CCR8 (/) GeoMean |
| | AF647 (RL1-H) of |
| | CHO-K1human |
| | CCR8 in |
| ScFv name | H12(NC) |
| WT | 2.2 |
| VH-PH7-P1C7 | 0.9 |
| VH-PH7-P2H12 | 6 |
| VH-PH5-P2B6(LCDR3) | 4.6 |
| VH-PH5-P3E3 | 3.6 |
| VH-PH5-P5F7 | 7.1 |
| VH-PH5-P6C8 | 1.4 |
| VH-PH5-P7B9 | 10.4 |
| VH-PH5-P9E9 | 10.8 |
| VL-PH5-P1D12 | 1.8 |
| VL-PH5-P10F1 | 1.2 |
| VL-PH5-P3E4 | 2.9 |
| VL-PH5-P5D9 | 3.2 |
| VL-PH5-P8G7 | 2.4 |
| VL-PH5-P9H3 | 1.9 |

TABLE 20
IgG production of 149 affinity matured variants

| | HCDR3 | LCDR3 |
|---|---|---|
| 149-11 | VH-PH5-P7B9 | WT |
| 149-12 | VH-PH5-P9E9 | WT |
| 149-13 | VH-PH5-P5F7 | WT |
| 149-14 | VH-PH5-P3E3 | WT |
| 149-15 | VH-PH7-P2H12(S41P) | WT |
| 149-16 | WT | VH-PH5-P2B6(LCDR3) |
| 149-17 | WT | VL-PH5-P5D9 |
| 149-18 | WT | VL-PH5-P3E4 |
| 149-19 | VH-PH5-P7B9 | VH-PH5-P2B6(LCDR3) |
| 149-20 | VH-PH5-P9E9 | VH-PH5-P2B6(LCDR3) |
| 149-21 | VH-PH5-P5F7 | VH-PH5-P2B6(LCDR3) |
| 149-22 | VH-PH5-P3E3 | VH-PH5-P2B6(LCDR3) |
| 149-23 | VH-PH5-P7B9(S41P) | VH-PH5-P2B6(LCDR3) |
| 149-24 | VH-PH5-P9E9(S41P) | VH-PH5-P2B6(LCDR3) |

### Example 17

### Binding and Blocking Activities of Selected Affinity Matured Antibodies

The binding affinities of affinity matured antibodies Hu149 were compared with those of parental antibody by FACS analysis (Sartourius, iQue3) using CHO-K1-human CCR8 and HEK293-cynomolgus CCR8 cells. And the blocking activities of affinity matured antibodies Hu149 were evaluated on CHO-K1-human CCR8 cell lines. As shown in Table 21, all the tested affinity matured antibodies showed comparable binding affinities and blocking activities on CHO-K1-human CCR8 and HEK293-cynomolgus CCR8 cells except for Hu149-21.

TABLE 21
Activities of affinity matured antibodies on CHO-
K1-human CCR8 and HEK293-Cynomolgus CCR8 cells.

| | | | | | Blocking | |
|---|---|---|---|---|---|---|
| | Binding | | Binding | | on | |
| | on | | on | | CHO-K1- | |
| | CHO-K1- | | HEK293- | | human | |
| | human | | Cynomolgus | | CCR8 | |
| | CCR8 | | CCR8 | | | Max |
| | EC₅₀ | Max | | Max | | Inhibition |
| Antibody | (nM) | MFI | EC₅₀ | MFI | IC₅₀ (nM) | (%) |
| 433H | 0.0823 | 814282 | 19.380 | 1719063 | 0.114 | 100 |
| Hu149-9 | 0.0487 | 625162 | 0.0824 | 3531299 | 0.0870 | 99.2 |
| Hu149-11 | 0.0797 | 647698 | 0.137 | 4596368 | 0.0988 | 100 |
| Hu149-12 | 0.0675 | 638366 | 0.199 | 4043870 | 0.0982 | 100 |
| Hu149-13 | 0.100 | 653232 | 0.193 | 4372697 | 0.0841 | 102 |
| Hu149-14 | 0.0626 | 663292 | 0.157 | 4422230 | 0.0816 | 100 |
| Hu149-15 | 0.128 | 626568 | 0.282 | 3930592 | 0.104 | 99.3 |
| Hu149-16 | 0.0659 | 672341 | 0.142 | 3795525 | 0.114 | 101 |
| Hu149-17 | 0.0755 | 645028 | 0.123 | 4106173 | 0.0698 | 100 |
| Hu149-18 | 0.0722 | 693731 | 0.0826 | 4154853 | 0.0614 | 101 |
| Hu149-19 | 0.161 | 666127 | 0.214 | 3945242 | 0.168 | 100 |
| Hu149-20 | 0.129 | 751331 | 0.158 | 4017715 | 0.147 | 100 |
| Hu149-21 | 0.642 | 793664 | 0.946 | 4149542 | 0.796 | 101 |
| Hu149-22 | 0.216 | 792907 | 0.462 | 4248584 | 0.459 | 101 |
| Hu149-23 | 0.265 | 815511 | 0.369 | 4322867 | 0.259 | 103 |
| Hu149-24 | 0.166 | 844323 | 0.161 | 3901317 | 0.172 | 106 |
| Human | NA¹ | 5137 | NA¹ | 14331 | NA¹ | −4.58 |
| IgG1 | | | | | | |

¹Not Applicable

HuT78 cells expressing endogenous human CCR8 were used for determining binding affinities of selected affinity matured anti-CCR8 antibodies. Briefly, a total of S5×10⁴ cells for each well were seeded in 96-well plates and washed with FACS buffer (DPBS containing 1.500 FBS) for once. Antibodies were prepared with 4-fold serial dilution ranging from 200 nM to 0.0122 nM in FACS buffer. Cells were incubated with 50 μL of diluted antibodies at 4° C. for 1 hour. After primary antibody incubation, cells were washed for 3 times by FACS buffer. Then, cells were stained by Alexa Fbu647 labeled anti-human IgG secondary antibody (Jackson ImmunoResearch, #109606170) at 1:600 dilution in FACS buffer for 0.5 hour at 4° C. After 3 times washed, flow cytometry (Sartourius, iQue3) was performed to measure the binding affinities of antibodies.

As shown in Table 22, Hu 149-11, Hu 149-12 and Hu 149-14 showed improved binding on HuT78 cells as compared to the parental antibody Hu149-9, the EC₅₀S of 2Hu49-11, Hu149-12 and Hu149-14 were 0.278 nM, 0.170 nM and 0.184 nM, respectively. As shown in Table 22, The Maximum MFI values of Hu149-16, Hu149-18, Hu149-19, Hu149-20, Hu149-21, Hu149-22, Hu149-23 and Hu149-24 were significantly higher than that of Hu149-9. Whether it was due to the non-specific binding need further investigation.

TABLE 22
Binding activities of affinity matured antibodies on HuT78 cells.

| | Binding on HuT78 | |
|---|---|---|
| Antibody | EC₅₀ (nM) | Max MFI |
| 433H | NA | 31945 |
| Hu149-9 | NA | 32320 |
| Hu149-11 | 0.278 | 30223 |
| Hu149-12 | 0.170 | 26933 |
| Hu149-13 | NA | 39136 |
| Hu149-14 | 0.184 | 28592 |
| Hu149-15 | 0.108 | 21300 |
| Hu149-16 | NA | 227789 |
| Hu149-17 | NA | 29558 |
| Hu149-18 | NA | 350693 |
| Hu149-19 | NA | 187113 |
| Hu149-20 | NA | 252665 |
| Hu149-21 | NA | 240325 |
| Hu149-22 | NA | 172785 |
| Hu149-23 | NA | 161166 |
| Hu149-24 | NA | 198285 |
| Human IgG1 | NA | 7821 |

### Example 18

### Binding of Selected Affinity Matured Antibodies on Human PBMCS

CCR8 is highly expressed in tumor-infiltrating regulatory T cells, but not in healthy T cells or effector T cells, so the PBMCs of healthy people can be used to detect non-specific binding of CCR8 antibody. Briefly, PBMCs were thawed and then blocked with Fc Blocker at 4° C. for 10 minutes, incubated with 200 nM antibody and PBMCs at 4° C. for 1 hour, and stained with FITC-anti-CD3, BV421-anti-CD4 and AF647-anti-human Fc at 4° C. for 30 minutes. The stained cells were analyzed by flow cytometry. Tables 23-24 show that Hu149-11 showed no or minimal binding on CD4+T and CD4- T cells as compared with the hIgG1 isotype control, while Hu149-18, Hu149-19, Hu149-20, Hu149-21, Hu149-22, Hu149-23 and Hu149-24 showed remarkable binding on CD4+T and CD4- T cells. Based on the FACS staining and gating strategy, CD4-T cells are considered as CD8+ T cells here.

TABLE 23
Flow cytometry analysis of Hu149-11 and Hu149-18-Hu149-24
binding on CD4+ T cells

| Cells | Antibody | MFI |
|---|---|---|
| BV421+, CD4+ | hlgG1 Isotype, 200 nM | 15.6 |
| BV421+, CD4+ | Hu149-24, 200 nM | 2157 |
| BV421+, CD4+ | Hu149-23, 200 nM | 144 |
| BV421+, CD4+ | Hu149-22, 200 nM | 283 |
| BV421+, CD4+ | Hu149-21, 200 nM | 120 |
| BV421+, CD4+ | Hu149-20, 200 nM | 2352 |
| BV421+, CD4+ | Hu149-19, 200 nM | 184 |
| BV421+, CD4+ | Hu149-18, 200 nM | 933 |
| BV421+, CD4+ | Hu149-11, 200 nM | 23.1 |
| BV421+, CD4+ | Hu433H, 200 nM | 17.5 |

TABLE 24
Flow cytometry analysis of Hu149-11 and Hu149-18-Hu149-24
binding on CD4− T cells

| Cells | Antibody | MFI |
|---|---|---|
| BV421−, CD4− | hlgG1 Isotype, 200 nM | 33.9 |
| BV421−, CD4− | Hu149-24, 200 nM | 3819 |
| BV421−, CD4− | Hu149-23, 200 nM | 693 |
| BV421−, CD4− | Hu149-22, 200 nM | 1328 |
| BV421−, CD4− | Hu149-21, 200 nM | 757 |
| BV421−, CD4− | Hu149-20, 200 nM | 4512 |
| BV421−, CD4− | Hu149-19, 200 nM | 999 |
| BV421−, CD4− | Hu149-18, 200 nM | 2751 |
| BV421−, CD4− | Hu149-11, 200 nM | 83.9 |
| BV421−, CD4− | Hu433H, 200 nM | 45.2 |

### Example 19

### Affinity Matured Antibodies Inhibit CCL1 Induced B-Arrestin Recruitment

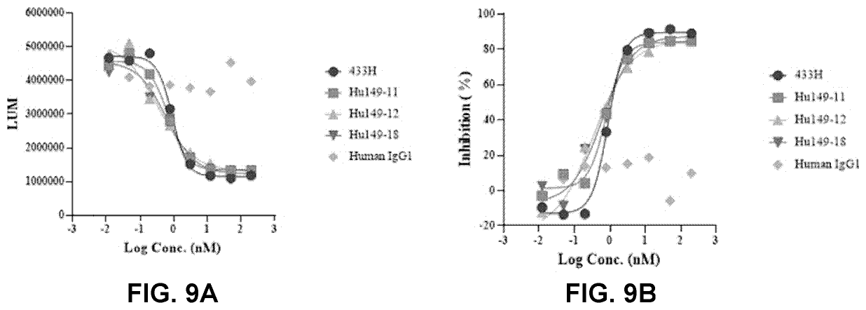

CHO-K CCR8 β-arrestin cells were stimulated with CCL1 (CN-07, Almac) at EC₈₀ (4 nM) to activate β-Arrestin recruitment and selected humanized antibodies or reference antibodies were added to evaluate their ability to block activated β-Arrestin recruitment. As shown in FIG. 9 and Table 25, Hu149-11, Hu149-12, Hu149-18 and 433H showed similar inhibitory activities on 03-Arrestin recruitment and the IC₅₀s were 0.800 nM, 0.350 nM, 0.610 nM and 0.890 nM, respectively.

TABLE 25
Activities of matured antibodies in inhibiting CCL1 induced
β-Arrestin recruitment.

| | CHO-K1-human CCR8 β-Arrestin | |
|---|---|---|
| Antibody | IC₅₀(nM) | Max Inhibition (%) |
| 433H | 0.890 | 92.5 |
| Hu149-11 | 0.800 | 85.2 |
| Hu149-12 | 0.350 | 85.8 |
| Hu149-18 | 0.610 | 88.8 |
| Human IgG1 | NA | 30.7 |

Th term “NA” indicates “Not Applicable”.

### Example 20

### Affinity Matured Antibodies Inhibit Ccl 1 Induced Cell Migration

Activities of matured anti-CCR8 antibodies in blocking CCL1 induced cell migration were evaluated. CHO-K1-human CCR8 cells with or without anti-CCR8 antibodies were added into upper chamber of transwell-96 permeable support. Chemoattractant, CCL1 was added into lower chamber. Cells passed through the membrane from the top and stick on the back side of the membrane. Cells were dissociated from the membrane using Trypsin and quantitated by Cell titer Glo.

$$Inhibition rate ( \% ) = ( 1 - \frac{chem o taxis index ( sample ) - 1}{c h e m o taxis ( without antibody ) - 1} ) \times 100. \\ Chemotaxis index = \frac{CTG va1ue ( samples )}{CTG va1ue ( without CCL 1 )}$$

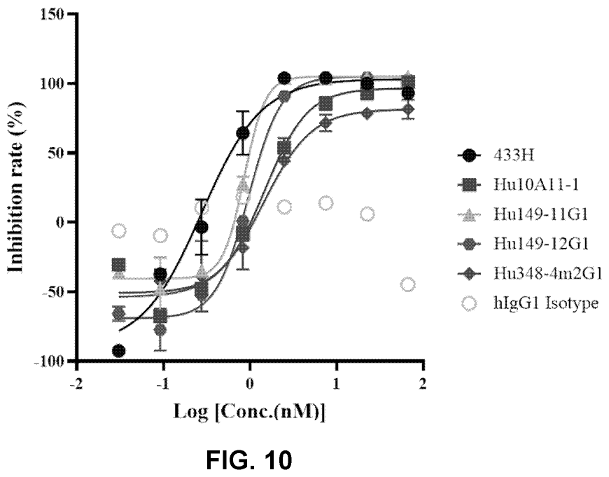

As shown in FIG. 10, Hu149-11G1, Hu149-12G1 and Hu348-4m2G1 were tested in migration assay. 433H was used as a positive control. Hu149-11G1, Hu149-12G1 and Hu348-4m2G1 blocked the cell migration induced by CCL1. The IC₅₀ s were 0.849 nM, 0.953 nM, and 1.42 nM, respectively (Table 26).

TABLE 26
Activities of anti-CCR8 antibody in blocking of cell migration
induced by CCL1

| Antibody | IC₅₀ (nM) | Max Inhibition (%) |
|---|---|---|
| Hu149-11G1 | 0.849 | 105 |
| Hu149-12G1 | 0.953 | 104 |
| Hu348-4m2G1 | 1.42 | 81.5 |
| Hu10A11-1 | 1.47 | 101 |
| 433H | 0.278 | 104 |

### Example 21

### Binding and Blocking Activities of Fc Variants of Humanized Anti-CCR8 Antibodies

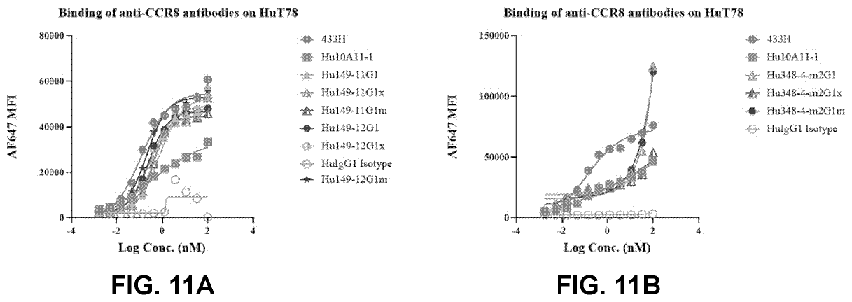

The binding and blocking activities of humanized anti-CCR8 antibodies with different Fc variants were evaluated on CHO-K1-human CCR8 cell line and its cross reaction on HEK293-Cynomolgus CCR8 cell lines described above. As shown in Table 27, Hu149-11G1m and Hu149-11G1x showed similar binding and blocking affinities to their parental antibody Hu149-11G1. Besides, Hu348-4-m2G1m and Hu348-4-m2G1x also showed similar binding and blocking activities compared to their parental antibody Hu348-4-m2G1. Hu149-11G1m, Hu149-11G1x, Hu348-4-m2G1m and Hu348-4-m2G1x also showed better binding activities compared with Hu10A11-1 as their parental antibodies on CHO-cynomolgus CCR8 cells. As shown in FIG. 11 and Table 28, Hu149-11G1m and Hu149-11G1x also showed better binding activities and higher binding signal compared to Hu10A11-1 on HuT78 cells. Hu348-4-m2G1m and Hu348-4-m2G1x also showed much better binding activities compared with Hlu10A11-1 on HuT78 cells.

TABLE 27
Activities of Fc variants of humanized anti-CCR8 antibodies
on CHO-K1-human CCR8 and HEK293-Cynomolgus CCR8 cells

| | | | | | Blocking | |
|---|---|---|---|---|---|---|
| | Binding | | Binding | | on | |
| | on | | on | | CHO-K1- | |
| | CHO-K1- | | HEK293- | | human | |
| | human | | Cynomolgus | | CCR8 | |
| | CCR8 | | CCR8 | | | Max |
| | EC₅₀ | Max | EC₅₀ | Max | IC₅₀ | Inhibition |
| Antibody | (nM) | MFI | (nM) | MFI | (nM) | (%) |
| 433H | 0.0699 | 214985 | NA | 648270 | 0.178 | 99.6 |
| Hu10A11-1 | 0.0657 | 179200 | NA | 824324 | 0.281 | 99.6 |
| Hu149-11G1 | 0.158 | 209835 | 0.262 | 4476251 | 0.283 | 99.5 |
| Hu149-11G1x | 0.115 | 200252 | 0.240 | 4515198 | 0.247 | 99.3 |
| Hu149-11G1m | 0.112 | 192621 | 0.217 | 4338810 | 0.231 | 99.4 |
| Hu149-12G1 | 0.0930 | 199408 | 0.170 | 3612341 | 0.184 | 99.5 |
| Hu149-12G1x | 0.0927 | 195134 | 0.150 | 3648319 | 0.170 | 100 |
| Hu149-12G1m | 0.0668 | 183027 | 0.183 | 3243922 | 0.146 | 99.6 |
| Hu348-4-m2G1 | 0.0133 | 149854 | 0.713 | 2129245 | 0.0543 | 95.4 |
| Hu348-4- | 0.0117 | 148576 | 0.230 | 1910784 | 0.0517 | 95.1 |
| m2G1x | | | | | | |
| Hu348-4- | NA | 131145 | NA | 1895103 | 0.0201 | 96.5 |
| m2G1m | | | | | | |
| HuIgG1 | NA | 3860 | NA | 5687 | NA | 17.8 |
| Isotype | | | | | | |

TABLE 28
Activities of Fc variants of humanized anti-CCR8
antibodies on HuT78 cells.

| | Binding on HuT78 | |
|---|---|---|
| Antibody | EC₅₀ (nM) | Max MFI |
| 433H | 0.123 | 60760 |
| Hu10A11-1 | 0.392 | 33372 |
| Hu149-11G1 | 0.735 | 57897 |
| Hu149-11G1x | 0.427 | 52740 |
| Hu149-11G1m | 0.384 | 45747 |
| Hu149-12G1 | 0.250 | 48059 |
| Hu149-12G1x | 0.313 | 52883 |
| Hu149-12G1m | 0.209 | 55595 |
| Hu348-4-m2G1 | NA | 124833 |
| Hu348-4-m2G1x | NA | 53900 |
| Hu348-4-m2G1m | NA | 120455 |
| HuIgG1 Isotype | NA | 16926 |

### Example 22

### ADCC Activities of Fc Variants of Humanized Anti-CCR8 Antibodies

The human FcγRIIIa displays a dimorphism in the position of residue 158. One allele (V158) encodes a higher Fc affinity receptor variant with a valine at amino acid residue 158, and the other (F158) encodes a lower Fc affinity receptor variant having a phenylalanine at amino acid residue 158. Jurkat T cells expressing firefly luciferase gene under the control of NFAT response elements and low affinity FcγRIIIa variant (F158 Cat #60540), Jurkat T cells expressing firefly luciferase gene under the control of NFAT response elements and high affinity FcγRIIIa variant (V158 Cat #60541) were purchased from BPS biosciences.

One day before the assay, the CD16a/NFAT-Jurkat cells (BPS 60640 or 60541) were seeded in assay medium and grew for 1 day. On the day of assay, 1×104 cells/well target cells in Assay Medium were seeded in a white opaque 96-well plate. Anti-CCR8 human antibody IgG1 or control antibody were added, mixed and incubated for 1 hour at 37° C. with 50° C.02. The CD16a/NFAT-reporter-Jurkat cells were harvested by centrifugation and resuspended in Assay Medium. 6×104 cells/well were added to the target cells and incubated with either anti-CCR8 or nonspecific negative control antibody. 100 μL Assay Medium were added into cell-free control wells (for determining background luminescence). Incubated the plates at 37° C. in a C02 incubator for 5 hours. After 5 hours incubation, 50 μl of ONE-Step Luciferase reagent per well were added and rocked gently at room temperature for 30 minutes. Measured luminescence using a luminometer. Data Analysis: Subtract the average background luminescence (cell-free control wells) from the luminescence reading of all wells. Nonlinear regression analysis was performed by GraphPad Prism 8 (GraphPad Software) and EC₅₀ values were calculated.

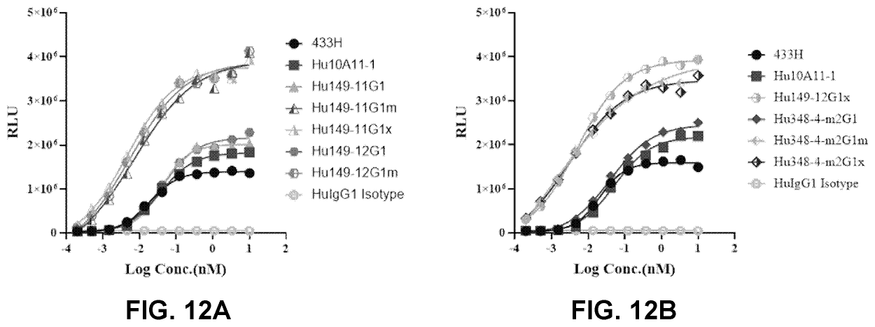

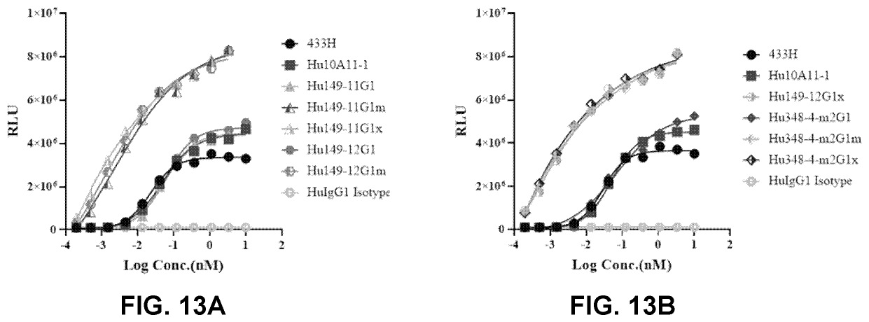

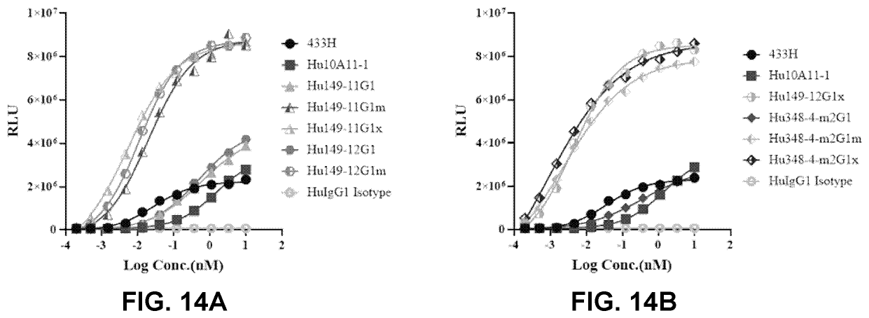

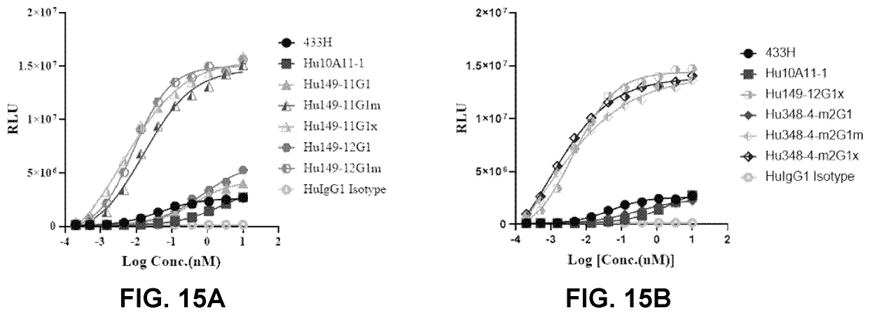

As shown in FIGS. 12-13, and Table 29, Hu149-11G1 (wild type variant) and Hu10A11-1 (wild type variant) showed similar ADCC activity with V or F variant CD16a/NFAT-Jurkat Cells on CHO-human CCR8 cells, which express high level of CCR8. However, Hu149-11G1 (wild type variant) was 2.5-5.5-fold more potent than Hu10A11-1 (wild type variant) as employing HuT78 cells as target cells, which express CCR8 endogenously and the CCR8 expression level is lower than that of CHO-CCR8. The EC₅₀s of Hu149-11G1 (wild type variant) and Hu10A11-1 (wild type variant) with V variant CD16a/NFAT-Jurkat cells on Hut78 were 0.586 nM and 1.47 nM, respectively. The EC₅₀s of Hu149-11G1 (wild type variant) and Hu10A11-1 (wild type variant) with F variant CD16a/NFAT-Jurkat cells on Hut78 were 0.402 nM, 2.20 nM, respectively (FIGS. 14-15, and Table 29). As shown in FIGS. 12-15 and Table 29, the ADCC activity of Hu149-11G1m (ADCC enhanced variant with L235V/F243L/R292P/Y300L/P396L Fc mutations) was much better than Hu149-11G1.

TABLE 29
ADCC activities of affinity matured antibodies with CD16a/NFAT-
Jurkat report cells on CHO-K1-human CCR8 and HuT78 cells.

| | V variant | | | | F variant | | | |
|---|---|---|---|---|---|---|---|---|
| | CD16a/NFAT-Jurkat | | | | CD16a/NFAT-Jurkat | | | |
| | CHO-K1-human | | | | CHO-K1-human | | | |
| | CCR8 | | HuT78 | | CCR8 | | HuT78 | |
| | EC₅₀ | | EC₅₀ | Max | EC₅₀ | Max | EC₅₀ | Max |
| Antibody | (nM) | Max (RLU) | (nM) | (RLU) | (nM) | (RLU) | (nM) | (RLU) |
| Hu433H | 0.0189 | 1413920 | 0.0281 | 2326720 | 0.0228 | 3518720 | 0.0440 | 2696480 |
| Hu10A11-1 | 0.0365 | 1832920 | 1.47 | 2784560 | 0.0503 | 4656440 | 2.20 | 2758600 |
| Hu149-11G1 | 0.0391 | 2026680 | 0.586 | 3886120 | 0.0586 | 4707200 | 0.402 | 4065280 |
| Hu149-11G1x | 0.00487 | 4087800 | 0.00559 | 9058480 | 0.00351 | 8335320 | 0.00460 | 15111160 |
| Hu149-11G1m | 0.00725 | 3934680 | 0.0203 | 8695800 | 0.00560 | 8281720 | 0.0164 | 15937440 |
| Hu149-12G1 | 0.0430 | 2282120 | 0.495 | 4186680 | 0.0598 | 4962160 | 0.744 | 5283080 |
| Hu149-12G1x | 0.00505 | 4130360 | 0.00458 | 8876560 | 0.00299 | 8274120 | 0.00428 | 15624400 |
| Hu149-12G1m | 0.00560 | 3935480 | 0.0114 | 8636920 | 0.00405 | 8176080 | 0.00869 | 14754920 |
| Hu348-4- | 0.0397 | 2506200 | 0.381 | 2473480 | 0.0563 | 5248840 | 0.270 | 2259480 |
| m2G1 | | | | | | | | |
| Hu348-4- | 0.00295 | 3933080 | 0.000780 | 7767720 | 0.00256 | 8134760 | 0.000860 | 13675680 |
| m2G1x | | | | | | | | |
| Hu348-4- | 0.000680 | 3574520 | 0.00238 | 8606120 | 0.00281 | 8083200 | 0.000410 | 14075080 |
| m2G1m | | | | | | | | |
| HuIgG1 | NA¹ | 55360 | NA¹ | 64840 | NA¹ | 114160 | NA¹ | 129320 |
| Isotype | | | | | | | | |

¹Not Applicable

Human PBMCs based ADCC activities of humanized antibodies were tested on CHO-K1 human CCR8 cells. Cryopreserved PBMCs (AllCells) of a healthy subject were thawed one day before the assay and cultured overnight in R-PMJ1640 medium with 10% FBS and 200 IU IL-2 (R&D, Cat #: 202-IL) in a C02 incubator. The target cells were labeled by CFSE (Life technology, Cat #: C34554) at the final concentration of 2.5 μM for 15 minutes. After staining, cell concentration was adjusted to 6×10⁴ cells/mL and mixed with 2-times volume of PBMCs which were adjusted to 1×10⁶ cells/mL (effector cell/target cell ratio was 40:1). Then, 150 μL of mixed target and effector cell suspension and 50 μL of series diluted antibodies were mixed in each well. Duplicate wells were prepared for each concentration of antibody. Target cells alone were used as control group. After incubation at 37° C., 50° C.02 for 5 hours, 1 μg/mL PI (Invitrogen, Cat #: 51-66211E) was added and analyzed by flow cytometry (BD FACS Celesta). Specific cytotoxicity was calculated by the formula: specific cytotoxicity=% PI positive cell with antibody- % PI positive cell without antibody.

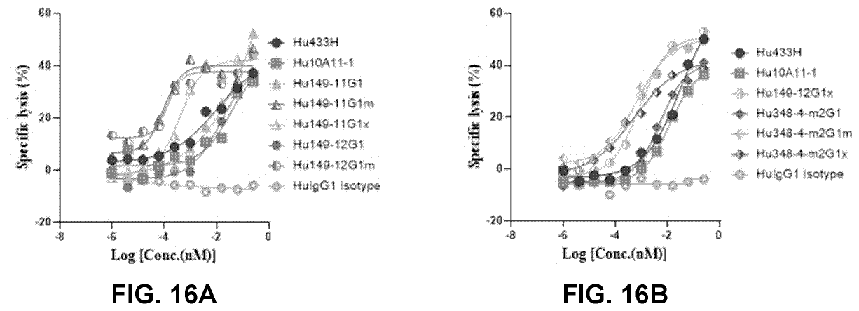

As shown in FIG. 16 and Table 30, Hu-149-11G1 (wild type variant) was more potent than 10A11-1 (wild type variant) in ADCC, the EC₅₀s were 0.0139 nM and 0.261 nM, respectively, and the maximum specific lysis rates were 52.5% and 34.0%, respectively. Hu149-11G1m (ADCC enhanced variant with L235V/F243L/R292P/Y300L/P396L Fc mutations) displayed remarkably improved ADCC activity against CHO-K1-human CCR8 cells compared to Hu149-11G1, the EC₅₀s were 0.0000850 nM and 0.0139 nM, respectively.

TABLE 30
ADCC activities of affinity matured antibodies with PBMCs on
CHO-K1-human CCR8 cells

| Antibody | EC₅₀(nM) | Max specific lysis (%) |
|---|---|---|
| Hu433H | 0.00395 | 37.3 |
| Hu10A11-1 | 0.0261 | 34.0 |
| Hu149-11G1 | 0.0139 | 52.5 |
| Hu149-11G1m | 0.0000850 | 46.6 |
| Hu149-11G1x | 0.000384 | 44.2 |
| Hu149-12G1 | 0.0113 | 36.4 |
| Hu149-12G1m | 0.000136 | 44.2 |
| Hu149-12G1x | 0.000828 | 53.0 |
| Hu348-4-m2G1 | 0.00483 | 41.1 |
| Hu348-4-m2G1m | 0.000608 | 50.7 |
| Hu348-4-m2G1x | 0.000516 | 40.5 |
| HuIgG1 | NA¹ | −1.90 |

¹Not Applicable

### Example 23

### Fc Variants of Humanized Anti-CCR8 Antibodies Inhibit Ccl 1 Induced B-Arrestin Recruitment

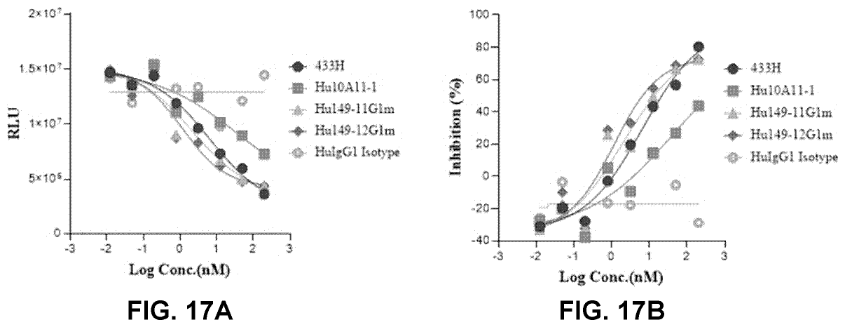

CHO-K1 CCR8 β-arrestin cells were stimulated with CCL1 (CN-07, Almac) at EC₈₀ (4 nM) to activate β-Arrestin recruitment, the selected humanized antibodies and reference antibodies were added to evaluate their ability in blocking activated β-Arrestin recruitment. As shown in FIG. 17 and Table 31, Hu149-11G1m was more potent than Hu10A11-1 in inhibiting β-Arrestin recruitment with IC₅₀s of 2.09 nM and 37.1 nM, and maximum inhibition rates of 72.7% and 43.6%, respectively. Hu149-11G1m and Hu149-12 G1m showed similar inhibitory activities of β-Arrestin recruitment, the IC₅₀ s were 2.09 nM and 1.16 nM, respectively.

TABLE 31
Activities of affinity matured antibodies in inhibiting CCL1
induced β-Arrestin recruitment

| | CHO-K1-human CCR8 β-Arrestin | |
|---|---|---|
| Antibody | IC₅₀(nM) | Max Inhibition (%) |
| 433H | 6.54 | 80.3 |
| Hu10A11-1 | 37.1 | 43.6 |
| Hu149-11G1m | 2.09 | 72.7 |
| Hu149-12G1m | 1.16 | 72.7 |
| HuIgG1 | NA¹ | 18.2 |

¹Not Applicable

### Example 24

### Binding of Fc Variants of Humanized Anti-CCR8 Antibodies on Human PBMCS

PBMCs of healthy people were used to detect non-specific binding of Fc variants of humanized anti-CCR8 antibodies. Briefly, PBMCs were thawed and then blocked with Fc Blocker at 4° C. for 10 minutes, incubated with 200 nM antibody and PBMCs at 4° C. for 1 hour, and stained with FITC-anti-CD3, BV421-anti-CD4 and AF647-anti-human Fc at 4° C. for 30 minutes. The stained cells were analyzed by flow cytometry. As shown in Table 32, Fc variants of Hu149-1 land Hu149-12 showed no or minimal binding on CD4+T and CD4- T cells. Hu348-4-m2G1m showed remarkable binding on CD4+T and CD4- T cells.

TABLE 32
Flow cytometry analysis of binding of Fc variants of humanized
anti-CCR8 antibodies on human CD4+ T cells

| Cells | Antibody | MFI |
|---|---|---|
| CD3+, CD4+ | hlgG 1 Isotype, 200 nM | 41.0 |
| CD3+, CD4+ | Hu348-4-m2G 1x, 200 nM | 78.1 |
| CD3+, CD4+ | Hu348-4-m2G 1m, 200 nM | 1645.0 |
| CD3+, CD4+ | Hu348-4-m2G 1, 200 nM | 77.5 |
| CD3+, CD4+ | Hu149-12G1x, 200 nM | 59.7 |
| CD3+, CD4+ | Hu10A11-1, 200 nM | 35.3 |
| CD3+, CD4+ | Hu433H, 200 nM | 16.0 |
| CD3+, CD4+ | Hu149-12G1m, 200 nM | 44.8 |
| CD3+, CD4+ | Hu149-12Gl, 200 nM | 31.5 |
| CD3+, CD4+ | Hu149-11G1x, 200 nM | 64.6 |
| CD3+, CD4+ | Hu149-11G1m, 200 nM | 38.2 |
| CD3+, CD4+ | Hu149-11G1, 200nM | 34.5 |
| CD3+, CD4+ | Hu10A11-1, 200 nM | 44.5 |
| CD3+, CD4+ | Hu433H, 200 nM | 14.3 |

### Example 25

### Expression of CCR8 on Different Immune Cell Populations

Frozen human dissociated tumor cells (hDTCs) from patients with kidney cancer, bladder cancer, breast cancer, colorectal cancer and melanoma were purchased from Discovery Life Sciences. Cells were thawed according to the instruction from the vendor and were filtered through a 70 um cell strainer. Cells were then counted and stained with Zombie Violet fixable viability dye (Biolegend, Cat #423114, 1:1000 in PBS) for 15 minutes at room temperature. Cells were washed with FACS staining buffer (Biolegend, Cat #420201) and 1-2 million/well hDTCs in 100 ul were seeded in each well of 96 well plate. hDTCs were first pre-stained with Human TruStain FcX (BioLegend, Cat #422302). Then Cells were surface stained, and intracellular staining was performed using the True-Nuclear transcription factor staining buffer Set (Biolegend, Cat #424401) using manufacturer's protocols. A FMO control was included for each hDTCs sample to ensure accurate gating. Samples were acquired using a BD LSRFortessa X-20 and analyzed using FlowJo (BD Biosciences). The complete list of flow cytometry antibodies can be found in Table 33.

TABLE 33
Complete list of flow cytometry antibodies
for hDTC immune phenotyping

| Target | Fluorophore | Clone number | catalog | Manufacturer |
|---|---|---|---|---|
| EpCAM+ | FITC | 9C4 | 324204 | Biolegend |
| CD45+ | BUV737 | HI30 | 748719 | BD biosciences |
| CD19+ | BV650 | HIB19 | 302238 | Biolegend |
| CD3+ | PerCP-Cy5.5 | SK7 | 344808 | Biolegend |
| CD4+ | BUV395 | SK3 | 563550 | BD biosciences |
| CD8+ | BV711 | RPA-T8 | 563676 | BD biosciences |
| CD56+ | APC | HCD56 | 318310 | Biolegend |
| CD11b+ | PE-CF594 | ICRF44 | 562399 | BD biosciences |
| CD25+ | PE-Cy7 | 2A3 | 335789 | BD biosciences |
| CCR8+ | BV605 | 433H | 747577 | BD biosciences |
| Foxp3+ | PE | 206D | 320108 | Biolegend |

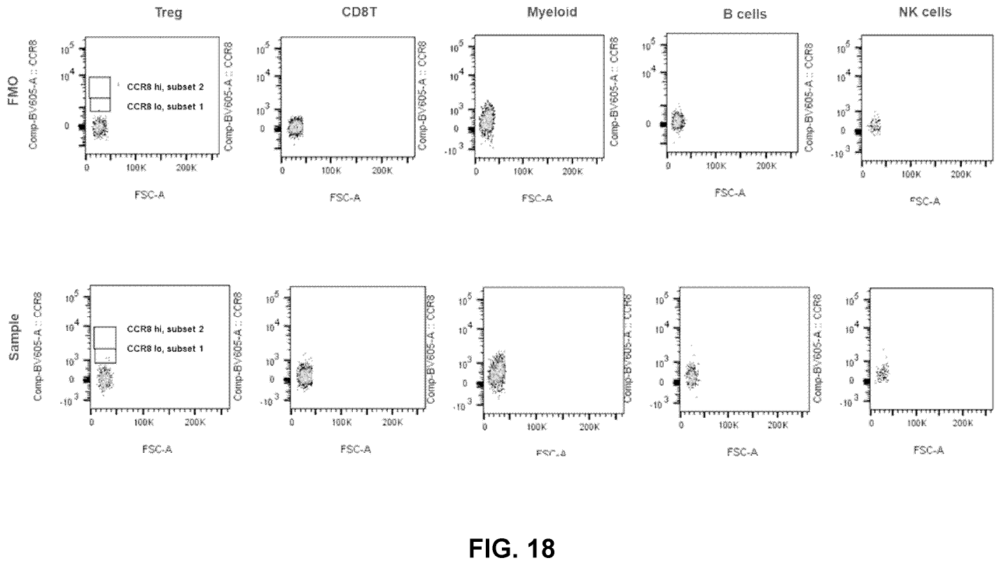

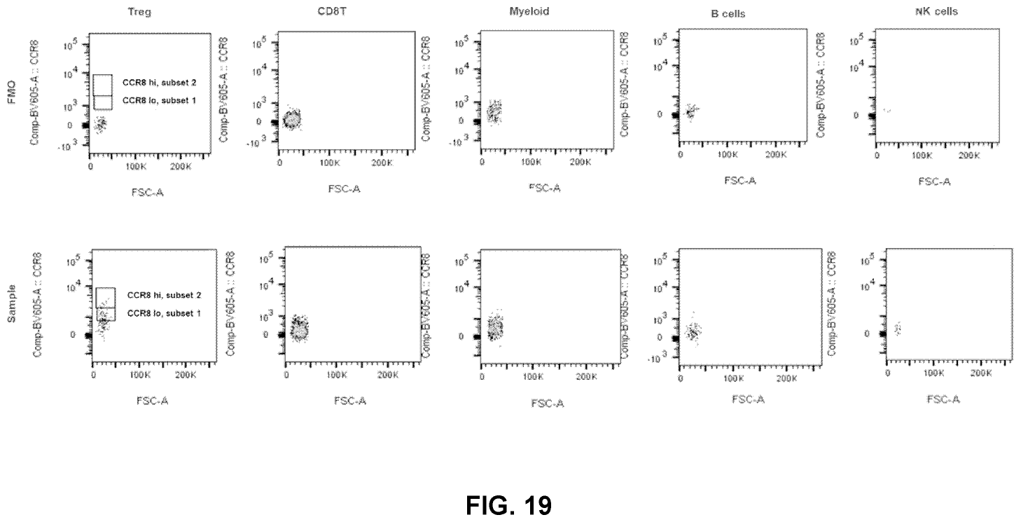

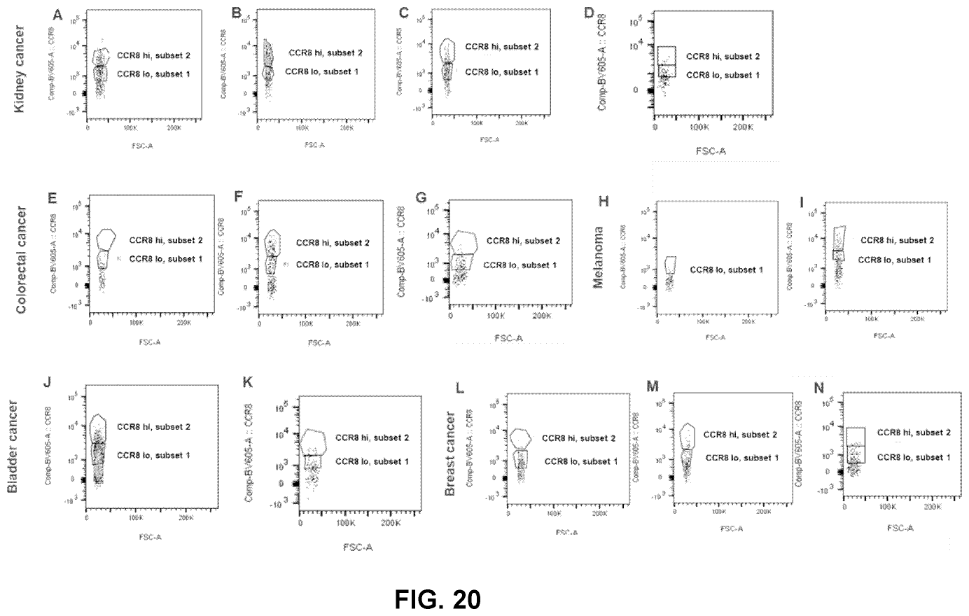

The expression pattern of the CCR8 protein in different immune cell populations using flow cytometry was evaluated. Experiment was done in both hDTCs and donor matched PBMCs. No CCR8 could be detected at the surface of major immune cell populations of PBMCs including FoxP3+ Treg cells, CD8+ T cells, CD11b+ myeloid cells, B cells and NK cells (FIG. 18). In contrast, a high CCR8 expression in the FoxP3+ Treg population of donor matched hDTCs (FIG. 19) was detected. CCR8 was marginally induced in CD8 T cells in hDTCs and was not detected in other populations including CD11b+ myeloid cells, B cells and NK cells. CCR8 was also not detected by Foxβ- conventional CD4+ T cells in either the peripheral blood or hDTCs. These data suggested that CCR8 is only upregulated in the Tregs in the tumor microenvironment, probably due to the TCR mediated activation of Tregs. By examining the CCR8 expression in hDTCs from different cancer types, two subsets of CCR8+ Tregs were observed: CCR8 lo (subset 1) and CCR8 hi (subset 2) populations. Within the same cancer type, Tregs in different donors showed heterogeneity: some donors have only subset 1 (FIG. 20, panel E, G, H, L, N) or both subset 1 and 2 (FIG. 20, panel A, B, C, D, F, I, J, K, M). It has been reported that CCR8 expression is positively associated with several activation markers and immune checkpoint molecules such as Lag3, K1rg1, Tnfrsf4, Tnfrsf9 and Il2ra, Helios, co-stimulatory molecule Cd81 and secretory molecules Areg and 1110 (1), indicating that the CCR8 hi Tregs (subset 2) probably have a highly suppressive phenotype.

### Example 26

### CCR8 Antibody Treatment Significantly Reduces Tumor Volume

Six- to eight-week-old female transgenic mice that had been engineered to express the human CCR8 gene in place of the murine CCR8 gene were purchased from Biocytogen (Wakefield, MA) and were acclimated for one week before the start of the study. The murine colorectal carcinoma cell line MC38 was implanted subcutaneously over the right flank of the mice at 0.5×10⁶ cells/100 μl/mouse. Prior to inoculation, the cells were cultured for no more than three passages in RPMI 1640 medium supplemented with 10% heat-inactivated Fetal Bovine Serum (FBS). Cells were grown at 37° C. in a humidified atmosphere with 5% C02. Upon reaching 80-85% confluence, cells were harvested and resuspended in a 1:1 mixture of serum-free RPMI 1640 and Matrigel at 5×10⁶ cells per milliliter.

Mice were monitored twice weekly following cell implantation for tumor growth. For tumor measurements, the length and width of each tumor was measured using calipers and volume was calculated according to the formula: Tumor volume (mm³)=(width (mm)×length (mm)²)/2. On the day of treatment initiation, all tumors were measured, outliers were excluded, and mice were randomly assigned to treatment groups. For anti-CCR8 treatment, a human IgG1 antibody was developed and engineered with an engineered (for enhanced ADCC activity) human IgG1 Fc. As a control, mice were administered a non-specific human IgG1 antibody with the same Fc mutation as the experimental antibody. Therapeutics were administered via intraperitoneal (i.p.) injection beginning 7 days after tumor inoculation on Study Day 0 when mean tumor volumes in each group were approximately 120 mm³. Treatment was subsequently delivered on Study Days 3, 7, and 11.

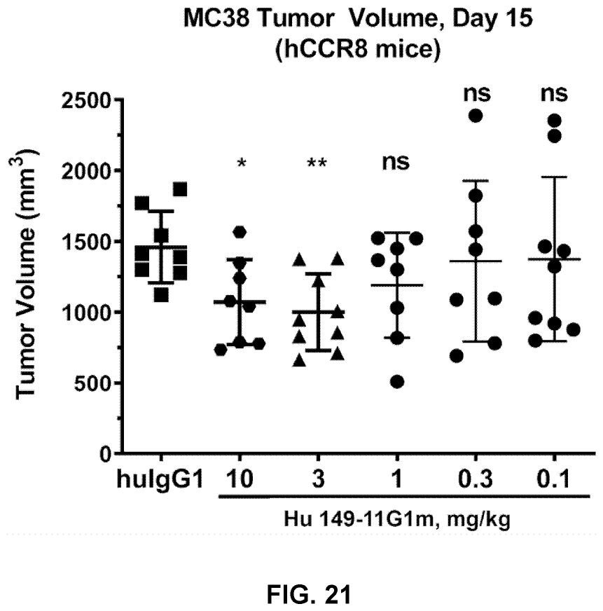

Tumors continued to be measured at least twice per week until tumor volume exceeded 10% of animal weight, or approximately 2000 mm³. The change in tumor size is shown by graphing individual tumor volumes for each treatment group on Day 15 after animals were first treated with Hu149-11G1m (FIG. 21). Treatment with Hu149-11G1m significantly reduced tumor growth compared to control IgG1 (p<0.05) when the therapeutic was administered at 3 or 10 mg/kg. Significant mean tumor growth inhibition was not observed for Hu149-11G1m when administered at 1, 0.3, or 0.1 mg/kg. β-values were calculated using unpaired, two-tailed t-test analyses of the calculated tumor volumes at the end of the study (Day 15). See FIG. 21, wherein statistical significance was determined via two-tailed, unpaired t-Test comparing Hu149-11G1m to human IgG1 control, with * p<0.05, ** p<0.01, and ns p>0.05.

### Example 27

Antitumor Efficacy of CCR8 Antibody in a Combination with Anti-PD-1 Antibody Against MC38 Tumor Model in HCCR8 Knockin Transgenic Mice

This study examined anti-tumor efficacy of a CCR8 antibody as a single agent and in a combination with a PD-1 antibody in vivo. More specifically, the efficacy of Hu149-11G1m in the human CCR8 transgenic mice that carry MC38 tumors was tested. The MC38 is a murine colon carcinoma cell line. Mice were inoculated subcutaneously with MC38, and tumor-bearing mice were then administered Hu149-11G1m twice per week at doses of 3 mg/kg as a single agent or in a combination with anti-PD-1 antibody (Biocell cat #CP151) at the doses of 5 mg/kg. Tumor growth was monitored throughout the study and the survival rate was recorded at the end of the study. The mice that achieved complete tumor regression were re-challenged with tumor to test the sustainability of the anti-tumor effects. As detailed below, in combo treatment group, a superior survival rate was achieved and a significant Treg depletion was observed.

MC38 murine colon cancer cells were cultured in DMEM medium supplemented with 10% FBS and 1x Penicillin/Streptomycin. After several passages in vitro, tumor cells were collected using TrypLE Express reagent, counted, and diluted to 10E06 cells per mL in PBS. An equal volume of Matrigel was added to the cell solution to bring the final concentration to 5E06 cells per mL.

Mice that are transgenic for the human gene were used in this study. Based on the C57B1/6 mouse strain, these animals had the human CCR8 gene inserted into the mouse CCR8 gene locus. Transgenic hCCR8 mice were inoculated by injecting into their right hind flank with 100 μL of cell suspension. Following 7 days of observation, mice were measured for tumor volume, and 10 animals were assigned to 4 treatment groups. The average tumor volume for each group was approximately 100 mm³. Tumor-bearing mice were injected twice per week with Hu149-11G1m 3mpk alone or in a combination with anti-PD-1. A human IgG₁ with matched Fc was included as a negative control (hIgG1 Control). Tumor volume (TV) was assessed twice per week using the formula TV=0.5×L×W×W until the tumor volume approached 1500 mm³ then the mouse was humanely euthanized.

On Day 105, 17 tumor eradicated mice were rechallenged with 5E05 MC38 tumor cells, in which 10 mice were from combo treated group and 7 mice from anti PD-1 treated group. The tumor growth was monitored until Day 131. All mice remained tumor free.

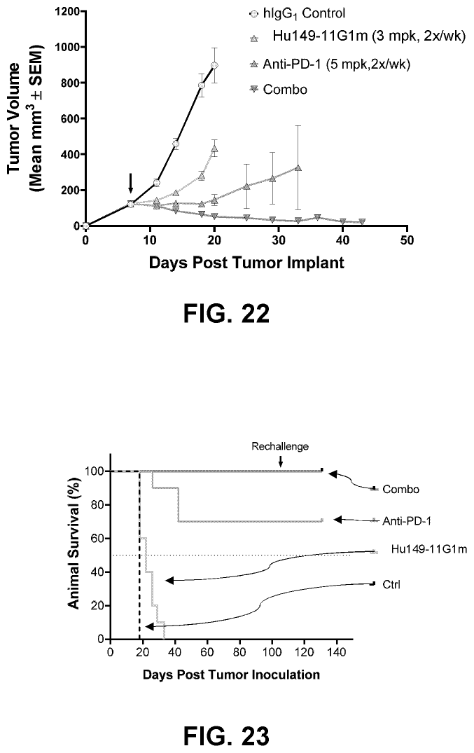

The results indicated that the MC38 tumors harvested from inoculated C57B1/6 transgenic mice did express human CCR8 on their Tregs. As shown in FIG. 22, treatment with Hu149-11G1m at 3 mg/kg showed a moderate anti-tumor response which was similar to the effects of anti-PD-1 antibodies. The combinational treatment of Hu149-11G1m at 3 mg/kg and anti-PD-1 at 5 mg/kg further reduced the mean tumor volume and such anti-tumor effects sustained after the cessation of drug treatment. The mice with complete tumor regression did not have any tumor growth 15 weeks after the re-challenge of the tumor (FIG. 22). The combo treatment group displayed superior survival rate, as shown in FIG. 23. Importantly, the results also showed that there was no substantive mean body weight loss in any of the groups treated with Hu149-11G1m alone or in a combination with anti-PD-1 antibodies. Together, these data demonstrate that CCR8 antibody such as Hu149-11G1m can mediate antitumor activity in vivo, and furthermore, render sensitivity to anti-PD-1 antibodies. The anti-tumor effects are sustainable.

### Example 28

Antitumor Activity of CCR8 Antibody in a Combination with Anti-PD-1 Antibody in HCCR8 Knockin Transgenic Mice Inoculated with E0771 Tumors

In this study, immunocompetent mice that engineered with human CCR8 gene in place of the murine gene were inoculated with murine E0771 breast cancer cells. Tumor-bearing mice were treated with either Hu149-11G1m alone, or in a combination with anti-PD-1 antibody (Biocell cat #CP151), and tumor growth was assessed.

Human gene knockin transgenic mice was used in the current study. Based on the C57Bl/6 mouse strain, these animals had the human CCR8 gene inserted into the mouse CCR8 gene locus. The E0771 cell line is a spontaneously developing medullary breast adenocarcinoma from C57BL/6 mice. hCCR8 transgenic C57BL/6 mice were inoculated with the E0771 mouse breast cancer cell line, and tumor-bearing mice were then administered with Hu149-11G1m twice per week at doses of 3 mg/kg as a single agent or in a combination with anti-PD-1 antibody at the doses of 5 mg/kg. Tumor growth was monitored throughout the study and the survival rate was recorded at the end of the study. The assessment of Tumor Infiltrating Lymphocytes (TIL profiling) was also performed to understand how Hu149-11G1m affected the immune cells in the tumor microenvironment (TME). After dissociating fresh tumors, CD4, CD8 and Tregs were evaluated.

E0771 murine breast cancer cells were cultured in RPMI medium supplemented with 10% FBS and 1x Penicillin/Streptomycin. After several passages in vitro, tumor cells were collected using TrypLE Express reagent, counted, and diluted to 20E06 cells per mL in PBS. An equal volume of Matrigel was added to the cell solution to bring the final concentration to 10E06 cells per mL.

Transgenic hCCR8 mice were inoculated by injecting into their right mammary fat pad with 50 μL of cell suspension. Following 7 days of observation, mice were measured for tumor volume, and 56 animals were assigned to 4 treatment groups (14 mice each). The average tumor volume for each group was approximately 80 mm³. Tumor-bearing mice were injected twice per week with Hu149-11G1m, anti-PD-1 at the denoted dose levels or human IgG1 at 10 mg/kg. Tumor volume was assessed twice per week using the formula TV=0.5×L×W×W until the tumor volume approached 1500 mm³ then the mouse was humanely euthanized.

On 7 days post tumor implantation, a subset group of mice (n=4/treatment group) were selected to harvest tumor tissue and perform TIL profiling. Tumor tissue was homogenized and digested with Tumor Dissociation Kit (Miltenyi 130-096-730). 3 million cells from each sample were stained and run flow cytometry analysis.

The rest of the mice (n=10/treatment group) continued to be monitored for a health check and tumor volume measurement twice a week. All the mice without excessive tumor burden (TV≥1500 mm³) would be maintained for the assessment of survival. As detailed below, in combo treatment group, a better survival rate was achieved and a significant Treg depletion was observed

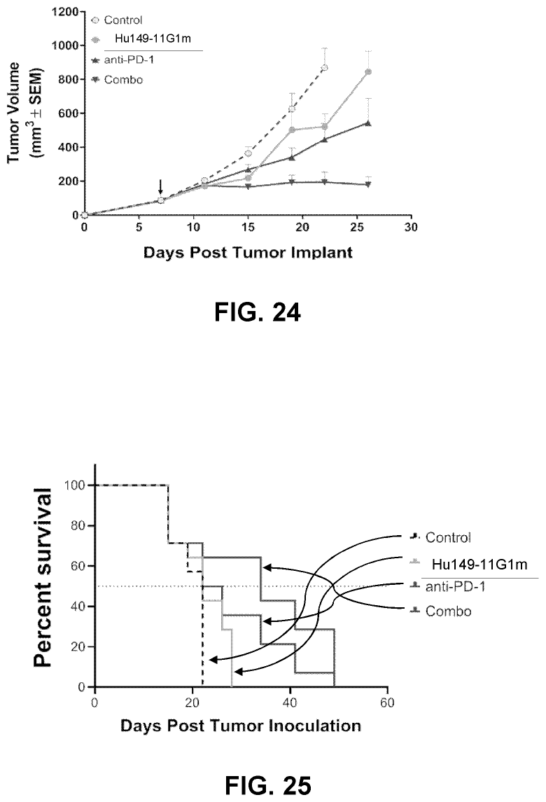

Result: The combinational treatment of Hu149-11G1m at 3 mg/kg and anti-PD-1 at 5 mg/kg further reduced the mean tumor volume and such anti-tumor effects sustained after the cessation of drug treatment (See FIG. 24). Table 34 illustrates the tumor volume from individual animals on Day 22 post tumor inoculation, the treatment of Hu149-11G1m as a single agent and in a combination with anti-PD-1 antibody resulted in a significant reduction in tumor growth (p<0.05 and p<0.001) compared to the negative control (a human IgG₁ with matched Fc). Moreover, at the later timepoint on Day 26 post tumor inoculation (Table 35), the combo treatment group displays significantly better anti-tumor effects than Hu149-11G1m and anti-PD-1 monotherapy group (p<0.01 and p<0.05). The combinational treatment also demonstrates survival benefits, shown in FIG. 25. Additionally, there was no substantive mean body weight loss in any of the groups treated with Hu149-11G1m alone or in a combination with anti-PD-1 antibodies. Together, these data demonstrate that CCR8 antibody, such as Hu149-11G1m, can mediate antitumor activity against tumors in vivo and furthermore, can render the sensitivity to anti-PD-1 antibodies.

TABLE 34
Tumor volumes and statistics on Day 22 post-treatment initiation

| | Number | | Std. Error | One way |
|---|---|---|---|---|
| Treatment | of Animals | Mean | of Mean | ANOVA |
| Control | 8 | 869.1 | 114.6 | NA |
| Hu149-11G1m | 9 | 522.3 | 75 | 0.04^{a} |
| Anti-PD-1 | 10 | 446.9 | 94.3 | 0.009^{b} |
| Combo | 10 | 194.6 | 60.1 | <0.001^{c} |

^{a}, ^{b}, and ^{c}comparing treated group to control;
NA: Not applicable

TABLE 35
Tumor volumes and statistics on Day 26 post-treatment initiation

| | Number | | Std. Error | |
|---|---|---|---|---|
| Treatment | of Animals | Mean | of Mean | t test^{a} |
| Hu149-11G1m | 6 | 845.7 | 121 | 0.006^{a} |
| Anti-PD-1 | 7 | 545.4 | 145 | 0.04^{b} |
| Combo | 9 | 178.8 | 49 | NA |

^{a}comparing Hu149-11G1m to combinational treated group;
^{b}comparing anti-PD-1 to combo treated group
NA: Not applicable.

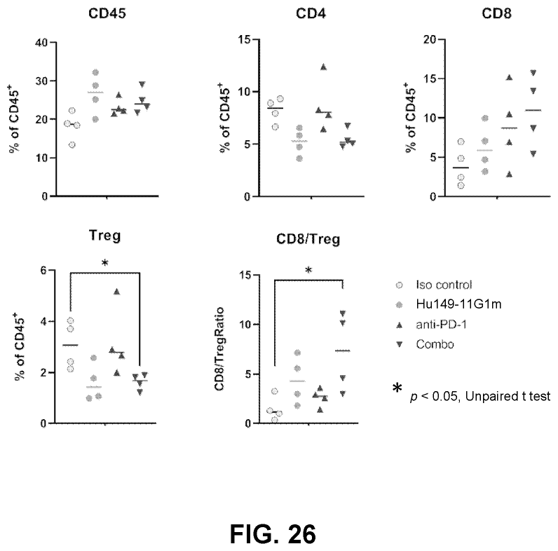

The current study also found that the administration of Hu149-11G1m and anti-PD-1 antibodies induced a significant depletion of Treg in the tumor and resulted in an increase in CD8+T/Treg ratio, FIG. 26 (p<0.05, four animals per treatment group were selected to harvest tumor tissue, immune cells were collected and stained for flow analysis). There is no significant change in other immune cell populations.

### Example 29

### Binding of Fc Variants of Humanized Anti-CCR8 Antibodies on Tall-1 Cells

TALL-1 is a human T cell leukemia cell line that endogenously expresses CCR8 at a level that is comparable to that observed in tumor-infiltrating Treg cells and was further used for investigating binding activities of Fc variants of humanized anti-CCR8 antibodies. Briefly, a total of 5×10⁴ cells for each well were seeded in 96-well plates and washed with FACS buffer (DPBS containing 1.5% FBS) for once. Antibodies were prepared with 3-fold serial dilution ranging from 100 nM to 0.00508 nM in FACS buffer. Cells were incubated with 50 μL of diluted antibodies at 4° C. for 40 minutes. After primary antibody incubation, cells were washed twice by FACS buffer. Then, cells were stained by Alexa Flu647-labeled anti-human IgG Fc secondary antibody (Jackson ImmunoResearch, #109606170) at 1:500 dilution in FACS buffer for 40 minutes at 4° C. After 2 times wash with FACS buffer, flow cytometry (BD FACS Celesta) was performed to measure the binding affinities of antibodies.

As shown in Table 36, Hu149-11G1m, Hu149-11G1 (wild type Fc variant), and TPP21360 (wild type Fc variant) showed similar binding activities on TALL-1 cells, the EC₅₀s were 0.328 nM, 0.315 nM and 0.461 nM, respectively. The maximum MFI values of Hu149-11G1m, Hu149-11G1 (wild type variant), and TPP21360 (wild type variant, see WO2021152186) were also comparable.

TABLE 36
Binding activities of Fc variants of humanized 149
antibodies on TALL-1 cells

| Antibody | EC₅₀ (nM) | Maximum MFI |
|---|---|---|
| Hu149-11G1m | 0.328 | 107 |
| Hu149-11G1 | 0.315 | 111 |
| TPP21360 | 0.461 | 102 |
| HuIgG1-Glm Isotype | NA | 5.96 |

“NA” indicates “Not Applicable”

### Example 30

### ADCC Activities of Anti-CCR8 Antibodies

ADCC reporter bioassay was carried out to compare ADCC activities of anti-CCR8 antibodies against TALL-1 cells, which express CCR8 at a level that is comparable to that observed in tumor-infiltrating Treg cells. In this setting, anti-CCR8 antibodies, Hu149-11G1, Hu10A11-1, and TPP21360, all with wild type IgG1 Fc, and isotype control were employed in the study. The method was described previously (see Example 22). As shown in Table 37, Hu149-11G1 was significantly more potent than Hu10A11-1 and TPP21360 in the assays. The EC₅₀s of Hu149-11G1 were 0.0658 nM and 0.120 nM as using 158V CD16a/NFAT-Jurkat and 158F CD16a/NFAT-Jurkat cells as surrogate effector cells, respectively. The EC₅₀ values of Hu10A11-1 and TPP21360 were not applicable in the assays.

TABLE 37
ADCC activities of anti-CCR8 antibodies with CD16a/
NFAT-Jurkat report cells on TALL-1 cells

| | 158V CD16a/ | | 158F CD16a/ | |
|---|---|---|---|---|
| | NFAT-Jurkat | | NFAT-Jurkat | |
| | | Maximum | | Maximum |
| Antibody | EC₅₀ (nM) | (RLU) | EC₅₀ (nM) | (RLU) |
| Hu149-11G1 | 0.0658 | 650300 | 0.120 | 925980 |
| TPP21360 | NA | 99720 | NA | 171260 |
| Hu10A11-1 | NA | 180820 | NA | 184120 |
| HuIgG1 Isotype | NA | 57420 | NA | 143740 |

“NA” indicates “Not Applicable”

Human PBMCs-based ADCC assay was further conducted to compare ADCC activities of anti-CCR8 antibodies. Human PBMCs (AllCells) were cultured in RPMI 1640 medium containing 10% heat inactivated FBS and 200 IU IL-2 (R&D, Cat #: 202-IL) in T75 flasks overnight. In a 96-well plate, 2×104 TALL-1 cells were co-cultured with pre-activated PBMCs (at a ratio of 1:20) in the presence of antibodies at various concentrations, cells were incubated with RPMI 1640 containing 2% heat inactivated FBS for 4 hours. At the end of incubation, lysis buffer was added to the assay plate and incubated for 30 minutes, then LDH working solution (DOJINDO, Cat #: CK12) was added to the assay plate and incubated for 25 minutes. After adding the stop solution to the assay plate, absorbance values at 490 nm were measured by a microplate reader. Nonlinear regression analysis was performed by Prism 8 (GraphPad Software) and EC50 values were calculated (log(agonist) vs. response—Variable slope (four parameters)). Specific cytotoxicity was calculated by the formula: specific cytotoxicity %=(ER-ERO)/[(TMR-VCC)−(TSR-CMB)]×100. ER indicates experimental release; ERO indicates experimental release, no antibody; TMR indicates target cell maximal release; VCC indicates volume correction control; TSR indicates target cell spontaneous release; CMB indicates control of medium background.

As shown in Table 38, Hu-149-11G1 (wild type Fe variant) was 45.7-fold more potent than TPP21360 (wild type Fe variant) in ADCC assay, the EC50s were 0.0121 nM and 0.553 nM, respectively, and the maximum specific lysis rates were 13.8% and 10.2%, respectively. Hu149-11G1m (ADCC enhanced variant) displayed remarkably improved ADCC activity against TALL-1 cells as compared to Hu149-11G1 (wild type variant), the EC50 of Hu149-11G1m was 0.000682 nM, and the maximum specific lysis rate was 32.2%.

TABLE 38
ADCC activities of anti-CCR8 antibodies with
human PBMCs on TALL-1 cells

| | | Maximum specific lysis |
|---|---|---|
| Antibody | EC₅₀ (nM) | (%) |
| Hu149-11G1m | 0.000682 | 32.2 |
| Hu149-11G1 | 0.0121 | 13.8 |
| TPP21360 | 0.553 | 10.2 |
| HuIgG1-G1m Isotype | NA | 0.57 |

“NA” indicates “Not Applicable”

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An antigen binding unit, comprising: a light chain (LC) variable region and a heavy chain (HC) variable region,
wherein
the heavy chain variable region comprises HC-complementarity determining region (CDR11, HC-CDR2, and HC-CDR3,
the light chain variable region comprises LC-CDR1, LC-CDR2, and LC-CDR3, and
the HC-CDR1 comprises a sequence selected from SEQ ID NOs: 13, 71, 83, 115, and 117, the HC-CDR2 comprises a sequence selected from SEQ ID NOs: 14, 72, 77, 84, 89, 91, 92, 97, 114, 116, and 118, and the HC-CDR3 comprises a sequence selected from SEQ ID NOs: 15, 19-22, 73, 78, 81, 85, 93, 98, 112, and 113.

2.-8. (canceled)

9. The antigen binding unit of claim 1, wherein the LC-CDR1 comprises a sequence selected from SEQ ID NOs: 16, 74, 79, 86, 90, 94, 99, and 102-109, the LC-CDR2 comprises a sequence selected from SEQ ID NOs: 17, 75, 80, 87, 95, 100, 110, and 111, and the LC-CDR3 comprises a sequence selected from SEQ ID NOs: 18, 23-25, 76, 82, 88, 96, and 101.

10.-11. (canceled)

12. The antigen binding unit of claim 1, wherein the HC-CDR1, HC-CDR2, and HC-CDR3, as a group, comprise, respectively, amino acid sequences selected from the following groups: SEQ ID NO: 13, SEQ ID NO: 14, and SEQ ID NO: 15; SEQ ID NO: 13, SEQ ID NO: 14, and SEQ ID NO: 19; SEQ ID NO: 13, SEQ ID NO: 14, and SEQ ID NO: 20; SEQ ID NO: 13, SEQ ID NO: 14, and SEQ ID NO: 21; SEQ ID NO: 13, SEQ ID NO: 14, and SEQ ID NO: 22; SEQ ID NO: 71, SEQ ID NO: 72, and SEQ ID NO: 73; SEQ ID NO: 71, SEQ ID NO: 77, and SEQ ID NO: 78; SEQ ID NO: 71, SEQ ID NO: 72, and SEQ ID NO: 81; SEQ ID NO: 83, SEQ ID NO: 84, and SEQ ID NO: 85; SEQ ID NO: 13, SEQ ID NO: 89, and SEQ ID NO: 15; SEQ ID NO: 13, SEQ ID NO: 91, and SEQ ID NO: 15; SEQ ID NO: 71, SEQ ID NO: 92, and SEQ ID NO: 93; SEQ ID NO: 13, SEQ ID NO: 97, and SEQ ID NO: 98; SEQ ID NO: 13, SEQ ID NO: 97, and SEQ ID NO: 112; SEQ ID NO: 13, SEQ ID NO: 97, and SEQ ID NO: 113; SEQ ID NO: 13, SEQ ID NO: 114, and SEQ ID NO: 15; SEQ ID NO: 115, SEQ ID NO: 114, and SEQ ID NO: 15; SEQ ID NO: 83, SEQ ID NO: 116, and SEQ ID NO: 85; SEQ ID NO: 117, SEQ ID NO: 116, and SEQ ID NO: 85; and SEQ ID NO: 83, SEQ ID NO: 118, and SEQ ID NO: 85.

13. The antigen binding unit of claim 9, wherein the LC-CDR1, LC-CDR2, and LC-CDR3, as a group, comprise, respectively, amino acid sequences selected from the following groups: SEQ ID NO: 16, SEQ ID NO: 17, and SEQ ID NO: 18; SEQ ID NO: 16, SEQ ID NO: 17, and SEQ ID NO: 23; SEQ ID NO: 16, SEQ ID NO: 17, and SEQ ID NO: 24; SEQ ID NO: 16, SEQ ID NO: 17, and SEQ ID NO: 25; SEQ ID NO: 74, SEQ ID NO: 75, and SEQ ID NO: 76; SEQ ID NO: 79, SEQ ID NO: 80, and SEQ ID NO: 76; SEQ ID NO: 79, SEQ ID NO: 75, and SEQ ID NO: 82; SEQ ID NO: 86, SEQ ID NO: 87, and SEQ ID NO: 88; SEQ ID NO: 90, SEQ ID NO: 17, and SEQ ID NO: 18; SEQ ID NO: 94, SEQ ID NO: 95, and SEQ ID NO: 96; SEQ ID NO: 79, SEQ ID NO: 75, and SEQ ID NO: 76; SEQ ID NO: 99, SEQ ID NO: 100, and SEQ ID NO: 101; SEQ ID NO: 101, SEQ ID NO: 17, and SEQ ID NO: 18; SEQ ID NO: 103, SEQ ID NO: 17, and SEQ ID NO: 18; SEQ ID NO: 104, SEQ ID NO: 17, and SEQ ID NO: 18; SEQ ID NO: 105, SEQ ID NO: 17, and SEQ ID NO: 18; SEQ ID NO: 106, SEQ ID NO: 17, and SEQ ID NO: 18; SEQ ID NO: 107, SEQ ID NO: 100, and SEQ ID NO: 101; SEQ ID NO: 108, SEQ ID NO: 100, and SEQ ID NO: 101; SEQ ID NO: 109, SEQ ID NO: 100, and SEQ ID NO: 101; and SEQ ID NO: 99, SEQ ID NO: 111, and SEQ ID NO: 101.

14. The antigen binding unit of claim 12, wherein the HC-CDR1, HC-CDR2, HC-CDR3, LC-CDR1, LC-CDR2, and LC-CDR3, as group, comprise, respectively, amino acid sequences selected from the following groups: SEQ ID NOs: 13, 14, 15, 16, 17, and 18; SEQ ID NOs: 13, 14, 19, 16, 17, and 18, SEQ ID NOs: 13, 14, 20, 16, 17, and 18; SEQ ID NOs: 13, 14, 21, 16, 17, and 18; SEQ ID NOs: 13, 14, 22, 16, 17, and 18; SEQ ID NOs: 13, 14, 15, 16, 17, and 23; SEQ ID NOs: 13, 14, 15, 16, 17, and 24; SEQ ID NOs: 13, 14, 15, 16, 17, and 25; SEQ ID NOs: 13, 14, 19, 16, 17, and 23; SEQ ID NOs: 13, 14, 20, 16, 17, and 23; SEQ ID NOs: 13, 14, 21, 16, 17, and 23; SEQ ID NOs: 13, 14, 22, 16, 17, and 23; SEQ ID NOs: 13, 89, 15, 90, 17, and 18; SEQ ID NOs: 13, 89, 15, 102, 17, and 18; SEQ ID NOs: 13, 89, 15, 103, 17, and 18; SEQ ID NOs: 13, 89, 15, 104, 17, and 18; SEQ ID NOs: 13, 89, 15, 16, 17, and 18; SEQ ID NOs: 13, 89, 15, 105, 17, and 18; SEQ ID NOs: 13, 89, 15, 106, 17, and 18; SEQ ID NOs: 13, 114, 15, 16, 17, and 18; and SEQ ID NOs: 115, 114, 15, 16, 17, and 18.

15. The antigen binding unit of claim 12, wherein the HC-CDR1, HC-CDR2, HC-CDR3, LC-CDR1, LC-CDR2, and LC-CDR3, as a group, comprise, respectively, amino acid sequences selected from the following groups: SEQ ID NOs: 83, 84, 85, 86, 87, and 88; SEQ ID NOs: 83, 116, 85, 86, 87, and 88; SEQ ID NOs: 117, 116, 85, 86, 87, and 88; and SEQ ID NOs: 83, 118, 85, 86, 87, and 88.

16.-17. (canceled)

18. The antigen binding unit of claim 1, wherein the heavy chain variable region and the light chain variable region, as a group, comprise, respectively, amino acid sequences selected from the following groups: SEQ ID NOs: 1 and 2; SEQ ID NOs: 3 and 2; SEQ ID NOs: 4 and 2; SEQ ID NOs: 5 and 2; SEQ ID NOs: 6 and 2; SEQ ID NOs: 7 and 2; SEQ ID NOs: 1 and 8; SEQ ID NOs: 1 and 9; SEQ ID NOs: 1 and 10; SEQ ID NOs: 3 and 8; SEQ ID NOs: 4 and 8; SEQ ID NOs: 5 and 8; SEQ ID NOs: 6 and 8; SEQ ID NOs: 11 and 8; SEQ ID NOs: 12 and 8; SEQ ID NOs: 34 and 35; SEQ ID NOs: 34 and 43; SEQ ID NOs: 34 and 44; SEQ ID NOs: 34 and 45; SEQ ID NOs: 34 and 46; SEQ ID NOs: 34 and 47; SEQ ID NOs: 34 and 48; SEQ ID NOs: 56 and 2; SEQ ID NOs: 57 and 2; SEQ ID NOs: 58 and 2; SEQ ID NOs: 59 and 2; SEQ ID NOs: 56 and 60; SEQ ID NOs: 57 and 60; and SEQ ID NOs: 58 and 60; and SEQ ID NOs: 59 and 60.

19. The antigen binding unit of claim 1, wherein the heavy chain variable region and the light chain variable region, as a group, comprise, respectively, amino acid sequences selected from the following groups: SEQ ID NOs: 32 and 33; SEQ ID NOs: 61 and 62; SEQ ID NOs: 61 and 63; SEQ ID NOs: 61 and 64; SEQ ID NOs: 61 and 65; SEQ ID NOs: 66 and 62; SEQ ID NOs: 67 and 62; SEQ ID NOs: 68 and 62; SEQ ID NOs: 69 and 62; and SEQ ID NOs: 70 and 62.

20. The antigen binding unit of claim 1, wherein the antigen binding unit is a monoclonal antibody, a humanized antibody or a chimeric antibody.

21. The antigen binding unit of claim 1, wherein the antigen binding unit is a scFv, Fab′, a single chain Fab (scFab′), a Fd or a F(ab′)₂.

22. The antigen binding unit of claim 1, which comprises an IgG1 framework.

23. The antigen binding unit of claim 22, which comprises one or more mutations in the IgG1 framework Fc region, optionally wherein the one or more mutations comprises at least one of a S239D mutation, a 1332E mutation, a L235V mutation, a F243L mutation, a R292P mutation, a Y300L mutation, and a P396L mutation.

24.-25. (canceled)

26. The antigen binding unit of claim 1, wherein the antigen binding unit binds at least one of a human C—C motif chemokine receptor 8 (CCR8), and a cynomolgus CCR8, or blocks binding of chemokine C—C motif ligand 1 (CCL1) to the human CCR8 or cynomolgus CCR8.

27. (canceled)

28. A pharmaceutical composition comprising the antigen binding unit of claim 1, and a pharmaceutically acceptable excipient.

29. An isolated nucleic acid encoding the antigen binding unit of claim 1.

30. A vector comprising a nucleic acid sequence encoding the antigen binding unit of claim 1.

31. (canceled)

32. A host cell comprising a nucleic acid encoding the antigen binding unit of claim 1.

33. (canceled)

34. A method of treating a cancer in a subject, comprising
administering to a subject in need thereof, an effective amount of the antigen binding unit of claim 1;
optionally wherein the antigen binding unit is administered at a dosage ranging from about 0.1 mg/kg to about 10 mg/kg.

35.-36. (canceled)

37. The method claim 34, where the cancer is a colorectal cancer (CRC), a breast cancer, an ovarian cancer, a non-small-cell lung cancer (NSCLC), a melanoma, a kidney cancer, a bladder cancer, or a sarcoma.

38. (canceled)

39. The method of claim 34, further comprising administering at least one more therapeutic antibody to the subject, optionally the at least one more therapeutic antibody is an anti-PD-1 antibody, an anti-PD-L1 antibody, or an anti-CLTA-4 antibody.

40.-50. (canceled)


SEQUENCE LISTING (121 sequences)

SEQ ID NO: 1 - protein, 123 aa, synthetic construct
EVQLVESGGGLVQPGGSLKLSCAASGFTFNTYAMNWVRQASGKGLEWVGRIRSKSNFYATAYAASVKGRFTISRDDSKNTAYLQMNSLKTEDTAVYYCTRGRDYGSSYAMDYWGQGTLVTVSS

SEQ ID NO: 2 - protein, 112 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHLEYPLTFGGGTKVELK

SEQ ID NO: 3 - protein, 123 aa, synthetic construct
EVQLVESGGGLVQPGGSLKLSCAASGFTFNTYAMNWVRQASGKGLEWVGRIRSKSNFYATAYAASVKGRFTISRDDSKNTAYLQMNSLKTEDTAVYYCTRGREYGSSYAMDYWGQGTLVTVSS

SEQ ID NO: 4 - protein, 123 aa, synthetic construct
EVQLVESGGGLVQPGGSLKLSCAASGFTFNTYAMNWVRQASGKGLEWVGRIRSKSNFYATAYAASVKGRFTISRDDSKNTAYLQMNSLKTEDTAVYYCTRGKEYGSSYAMDYWGQGTLVTVSS

SEQ ID NO: 5 - protein, 123 aa, synthetic construct
EVQLVESGGGLVQPGGSLKLSCAASGFTFNTYAMNWVRQASGKGLEWVGRIRSKSNFYATAYAASVKGRFTISRDDSKNTAYLQMNSLKTEDTAVYYCTRGREYGRSYAMDYWGQGTLVTVSS

SEQ ID NO: 6 - protein, 123 aa, synthetic construct
EVQLVESGGGLVQPGGSLKLSCAASGFTFNTYAMNWVRQASGKGLEWVGRIRSKSNFYATAYAASVKGRFTISRDDSKNTAYLQMNSLKTEDTAVYYCTRGREYGSAYAMDYWGQGTLVTVSS

SEQ ID NO: 7 - protein, 123 aa, synthetic construct
EVQLVESGGGLVQPGGSLKLSCAASGFTFNTYAMNWVRQAPGKGLEWVGRIRSKSNFYATAYAASVKGRFTISRDDSKNTAYLQMNSLKTEDTAVYYCTRGRDYGSSYAMDYWGQGTLVTVSS

SEQ ID NO: 8 - protein, 112 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHLKYPLTFGGGTKVELK

SEQ ID NO: 9 - protein, 112 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHLSYPLTFGGGTKVELK

SEQ ID NO: 10 - protein, 112 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHLRYPLTFGGGTKVELK

SEQ ID NO: 11 - protein, 123 aa, synthetic construct
EVQLVESGGGLVQPGGSLKLSCAASGFTFNTYAMNWVRQAPGKGLEWVGRIRSKSNFYATAYAASVKGRFTISRDDSKNTAYLQMNSLKTEDTAVYYCTRGREYGSSYAMDYWGQGTLVTVSS

SEQ ID NO: 12 - protein, 123 aa, synthetic construct
EVQLVESGGGLVQPGGSLKLSCAASGFTFNTYAMNWVRQAPGKGLEWVGRIRSKSNFYATAYAASVKGRFTISRDDSKNTAYLQMNSLKTEDTAVYYCTRGKEYGSSYAMDYWGQGTLVTVSS

SEQ ID NO: 13 - protein, 5 aa, synthetic construct
TYAMN

SEQ ID NO: 14 - protein, 19 aa, synthetic construct
RIRSKSNFYATAYAASVKG

SEQ ID NO: 15 - protein, 12 aa, synthetic construct
GRDYGSSYAMDY

SEQ ID NO: 16 - protein, 16 aa, synthetic construct
RSSKSLLHSNANTYLY

SEQ ID NO: 17 - protein, 7 aa, synthetic construct
RMSNLAS

SEQ ID NO: 18 - protein, 9 aa, synthetic construct
MQHLEYPLT

SEQ ID NO: 19 - protein, 12 aa, synthetic construct
GREYGSSYAMDY

SEQ ID NO: 20 - protein, 12 aa, synthetic construct
GKEYGSSYAMDY

SEQ ID NO: 21 - protein, 12 aa, synthetic construct
GREYGRSYAMDY

SEQ ID NO: 22 - protein, 12 aa, synthetic construct
GREYGSAYAMDY

SEQ ID NO: 23 - protein, 9 aa, synthetic construct
MQHLKYPLT

SEQ ID NO: 24 - protein, 9 aa, synthetic construct
MQHLSYPLT

SEQ ID NO: 25 - protein, 9 aa, synthetic construct
MQHLRYPLT

SEQ ID NO: 26 - protein, 120 aa, synthetic construct
DVQLQESGPGLVKPSQSLSLTCSVTGYSITSGYYWNWIRQFPGNKLEWMGYISYDGSNNYNPSLKNRISITRDISKNQFFLKLNSVTTEDTATYYCARQIGLRRGAMDYWGQGTSVTVSS

SEQ ID NO: 27 - protein, 107 aa, synthetic construct
DIQMTQSSSYLSVSLGGRVTITCKASDHINTWLAWYQQKPGNAPRLLISGSTSLKAGVASRFSGSGSGKDYTLTITSLQTEDVATYYCQQYWGIPFTFGGGTKLEIK

SEQ ID NO: 28 - protein, 120 aa, synthetic construct
DVQLQESGPGLVKPSQSLSLTCSVTGYSITSGYYWNWIRQFPGNKLEWMGYTSYDGSNNYNPSFKNRISITRDISKNQFFLKLNSVTTEDTATYYCARQIGLRRGAMDHWGQGTSVTVSS

SEQ ID NO: 29 - protein, 107 aa, synthetic construct
DIQMTQSSSYLSVSLGGRVTITCKASDHINNWLAWYQQKPGNAPRLLISGSTSLRAGVASRFSGSGSGKDYTLTITSLQTEDVATYYCQQYWGIPFTFGGGTKLEIK

SEQ ID NO: 30 - protein, 120 aa, synthetic construct
DVQLQESGPGLVKPSQSLSLTCSVTGYSITSGYYWNWIRQFPGNKLEWMGYISYDGSNNYNPSLKNRISITRDISKNQFFLKLNSVTTEDTATYYCARQVGLRRGAMDYWGQGTSVTVSS

SEQ ID NO: 31 - protein, 107 aa, synthetic construct
DIQMTQSSSYLSVSLGGRVTITCKASDHINNWLAWYQQKPGNAPRLLISGSTSLKAGVASRFSGSGSGKDYTLSITSLQTEDVATYYCQQYWSIPYTFGGGTKLEIK

SEQ ID NO: 32 - protein, 117 aa, synthetic construct
QVQLVETGGGLVRPGNSLKLSCVTSGFSFSNYRMHWLRQPPGKRLEWIAVITVKSDNYGANYAESVKGRFTISRDDSKSSVYLQMNRLREEDTATYYCSSLGRVAYWGQGTLVTVSA

SEQ ID NO: 33 - protein, 107 aa, synthetic construct
DIVMTQSQKFMFTSVGDRVSVTCKASQNVDTNVVWYQQKPGQSPKALIYSASSRYSGVPDRFTGSGSGTDFTLTISNVQSEDLAEYFCQQHNSFPLTFGAGTKLELI

SEQ ID NO: 34 - protein, 123 aa, synthetic construct
EVQLVESGGGLVQPKGSLKLSCAASGFTFNTYAMNWVRQAPGKGLEWVARIRSKSNFYATYYADSVKDRFTISRDDSQDMLYLQMNILKTEDTAIYYCVRGRDYGSSYAMDYWGQGTSVTVSS

SEQ ID NO: 35 - protein, 112 aa, synthetic construct
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPLTFGAGTKLELK

SEQ ID NO: 36 - protein, 123 aa, synthetic construct
EVQLVESGGGLVQPKGSLKLSCAASGFTFNTYAMNWVRQAPGKGLEWVARIRSKSNYYATYYADSVKDRFTISRDDSQDMLYLQMNNLKTEDTAIYYCVRGRDYGSSYAMDYWGQGTSVTVSS

SEQ ID NO: 37 - protein, 120 aa, synthetic construct
DVQLQESGPGLVKPSQSLSLTCSVTGYSITSGYYWNWIRQFPGNKLEWMGYISYDGTNNYNPSLKNRISITRDTSKNQFFLKLTSVTTEDTATYYCARQVGLRRAWFAYWGQGTLVTVSA

SEQ ID NO: 38 - protein, 106 aa, synthetic construct
DIQMTQSSSFLSVSLGGRVTITCKASDHINYWLAWYQQKPGNAPRLLISGATSLETGVPSRFSGSGSGKDYTLTITSLQNEDVATYYCHQYWSTPTFGSGTKLEIK

SEQ ID NO: 39 - protein, 120 aa, synthetic construct
DVHLQESGPGLVKPSQSLSLTCSVTGYSITSGYYWNWIRQFPGNKLEWMGYISYDGSNNYNPSLKNRISITRDISKNQFFLKLNSVTTEDTATYYCARQIGLRRGAMDYWGQGTSVTVSS

SEQ ID NO: 40 - protein, 107 aa, synthetic construct
DIQMTQSSSYLSVSLGGRVTITCKASDHINNWLAWYQQKPGNAPRLLISGSTSLKAGVASRFSGSGSGKDYTLTITSLQTEDVATYYCQQYWGIPFTFGGGTKLEIK

SEQ ID NO: 41 - protein, 123 aa, synthetic construct
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRHGRSGYLYAMDYWGQGTSVTVSS

SEQ ID NO: 42 - protein, 112 aa, synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQSLELPLTFGAGTKLELK

SEQ ID NO: 43 - protein, 112 aa, synthetic construct
DIVMTQAAPSVPVTPGESVSISCRSSKSLLHSQGNTYLYWFLQRPGQSPQLLIYRMSNLASGVPDRFSGSGSGTAFTLRISRVEAEDVGVYYCMQHLEYPLTFGAGTKLELK

SEQ ID NO: 44 - protein, 112 aa, synthetic construct
DIVMTQAAPSVPVTPGESVSISCRSSKSLLHSIGNTYLYWFLQRPGQSPQLLIYRMSNLASGVPDRFSGSGSGTAFTLRISRVEAEDVGVYYCMQHLEYPLTFGAGTKLELK

SEQ ID NO: 45 - protein, 112 aa, synthetic construct
DIVMTQAAPSVPVTPGESVSISCRSSKSLLHSSGNTYLYWFLQRPGQSPQLLIYRMSNLASGVPDRFSGSGSGTAFTLRISRVEAEDVGVYYCMQHLEYPLTFGAGTKLELK

SEQ ID NO: 46 - protein, 112 aa, synthetic construct
DIVMTQAAPSVPVTPGESVSISCRSSKSLLHSNANTYLYWFLQRPGQSPQLLIYRMSNLASGVPDRFSGSGSGTAFTLRISRVEAEDVGVYYCMQHLEYPLTFGAGTKLELK

SEQ ID NO: 47 - protein, 112 aa, synthetic construct
DIVMTQAAPSVPVTPGESVSISCRSSKSLLHSNINTYLYWFLQRPGQSPQLLIYRMSNLASGVPDRFSGSGSGTAFTLRISRVEAEDVGVYYCMQHLEYPLTFGAGTKLELK

SEQ ID NO: 48 - protein, 112 aa, synthetic construct
DIVMTQAAPSVPVTPGESVSISCRSSKSLLHSNVNTYLYWFLQRPGQSPQLLIYRMSNLASGVPDRFSGSGSGTAFTLRISRVEAEDVGVYYCMQHLEYPLTFGAGTKLELK

SEQ ID NO: 49 - protein, 112 aa, synthetic construct
DIVMTQAAFSNPVTLGTSASISCRSSKSLLHSSGITYLYWYLQKPGQSPQLLIYQMSNLASGVPDRFSSSGSGTDFTLRISRVEAEDVGVYYCAQSLELPLTFGAGTKLELK

SEQ ID NO: 50 - protein, 112 aa, synthetic construct
DIVMTQAAFSNPVTLGTSASISCRSSKSLLHSYGITYLYWYLQKPGQSPQLLIYQMSNLASGVPDRFSSSGSGTDFTLRISRVEAEDVGVYYCAQSLELPLTFGAGTKLELK

SEQ ID NO: 51 - protein, 112 aa, synthetic construct
DIVMTQAAFSNPVTLGTSASISCRSSKSLLHSNAITYLYWYLQKPGQSPQLLIYQMSNLASGVPDRFSSSGSGTDFTLRISRVEAEDVGVYYCAQSLELPLTFGAGTKLELK

SEQ ID NO: 52 - protein, 112 aa, synthetic construct
DIVMTQAAFSNPVTLGTSASISCRSSKSLLHSNGITYLYWYLQKPGQSPQLLIYQASNLASGVPDRFSSSGSGTDFTLRISRVEAEDVGVYYCAQSLELPLTFGAGTKLELK

SEQ ID NO: 53 - protein, 112 aa, synthetic construct
DIVMTQAAFSNPVTLGTSASISCRSSKSLLHSNGITYLYWYLQKPGQSPQLLIYQTSNLASGVPDRFSSSGSGTDFTLRISRVEAEDVGVYYCAQSLELPLTFGAGTKLELK

SEQ ID NO: 54 - protein, 123 aa, synthetic construct
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRHGRSGYLYAADYWGQGTSVTVSS

SEQ ID NO: 55 - protein, 123 aa, synthetic construct
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRHGRSGYLYAFDYWGQGTSVTVSS

SEQ ID NO: 56 - protein, 123 aa, synthetic construct
EVQLVESGGGLVQPGGSLKLSCAASGFTFNTYAMNWVRQASGKGLEWVGRIRSKSNFYATYYAASVKGRFTISRDDSKNTAYLQMNSLKTEDTAVYYCVRGRDYGSSYAMDYWGQGTLVTVSS

SEQ ID NO: 57 - protein, 123 aa, synthetic construct
EVQLVESGGGLVQPGGSLKLSCAASGFTFNTYAMHWVRQASGKGLEWVGRIRSKSNFYATYYAASVKGRFTISRDDSKNTAYLQMNSLKTEDTAVYYCVRGRDYGSSYAMDYWGQGTLVTVSS

SEQ ID NO: 58 - protein, 123 aa, synthetic construct
EVQLVESGGGLVQPGGSLKLSCAASGFTFNTYAMNWVRQASGKGLEWVGRIRSKSNFYATAYAASVKGRFTISRDDSKNTAYLQMNSLKTEDTAVYYCVRGRDYGSSYAMDYWGQGTLVTVSS

SEQ ID NO: 59 - protein, 123 aa, synthetic construct
EVQLVESGGGLVQPGGSLKLSCAASGFTFNTYAMNWVRQASGKGLEWVGRIRSKSNFYATYYAASVKGRFTISRDDSKNTAYLQMNSLKTEDTAVYYCTRGRDYGSSYAMDYWGQGTLVTVSS

SEQ ID NO: 60 - protein, 112 aa, synthetic construct
DIVMTQSPLSLPVTPGEPASISCRSSKSLLHSNANTYLYWYLQKPGQSPQLLIYRMSNLASGVPDRFSGSGSGTDFTLKISRVEAEDVGVYYCMQHLEYPLTFGGGTKVELK

SEQ ID NO: 61 - protein, 117 aa, synthetic construct
EVQLVESGGGLVQPGGSLKLSCATSGFSFSNYRMHWLRQASGKGLEWVGVITVKSDNYGANYAASVKGRFTISRDDSKSSVYLQMNSLKTEDTAVYYCSSLGRVAYWGQGTLVTVSS

SEQ ID NO: 62 - protein, 107 aa, synthetic construct
DIQMTQSPSSVSASVGDRVTITCKASQNVDTNVVWYQQKPGQSPKALIYSASSRYSGVPDRFSGSGSGTDFTLTISSLQPEDFATYYCQQHNSFPLTFGQGTKLEIK

SEQ ID NO: 63 - protein, 107 aa, synthetic construct
DIQMTQSPSSVSASVGDRVTITCKASQNVDTNVVWYQQKPGKAPKALIYSASSRYSGVPDRFSGSGSGTDFTLTISSLQPEDFATYYCQQHNSFPLTFGQGTKLEIK

SEQ ID NO: 64 - protein, 107 aa, synthetic construct
DIQMTQSPSSVSASVGDRVTITCKASQNVDTNVVWYQQKPGQSPKLLIYSASSRYSGVPDRFSGSGSGTDFTLTISSLQPEDFATYYCQQHNSFPLTFGQGTKLEIK

SEQ ID NO: 65 - protein, 107 aa, synthetic construct
DIQMTQSPSSVSASVGDRVTITCKASQNVDTNVVWYQQKPGQSPKALIYSASSRYSGVPSRFSGSGSGTDFTLTISSLQPEDFATYYCQQHNSFPLTFGQGTKLEIK

SEQ ID NO: 66 - protein, 117 aa, synthetic construct
EVQLVESGGGLVQPGGSLKLSCAASGFSFSNYAMHWLRQASGKGLEWVGVITVKSDNYGANYAASVKGRFTISRDDSKSSVYLQMNSLKTEDTAVYYCSSLGRVAYWGQGTLVTVSS

SEQ ID NO: 67 - protein, 117 aa, synthetic construct
EVQLVESGGGLVQPGGSLKLSCATSGFSFSNYRMHWVRQASGKGLEWVGVITVKSDNYGANYAASVKGRFTISRDDSKSSVYLQMNSLKTEDTAVYYCSSLGRVAYWGQGTLVTVSS

SEQ ID NO: 68 - protein, 117 aa, synthetic construct
EVQLVESGGGLVQPGGSLKLSCATSGFSFSNYRMHWLRQASGKGLEWVGRITVKSDNYGTAYAASVKGRFTISRDDSKSSVYLQMNSLKTEDTAVYYCSSLGRVAYWGQGTLVTVSS

SEQ ID NO: 69 - protein, 117 aa, synthetic construct
EVQLVESGGGLVQPGGSLKLSCATSGFSFSNYRMHWLRQASGKGLEWVGVITVKSDNYGANYAASVKGRFTISRDDSKNTAYLQMNSLKTEDTAVYYCSSLGRVAYWGQGTLVTVSS

SEQ ID NO: 70 - protein, 117 aa, synthetic construct
EVQLVESGGGLVQPGGSLKLSCATSGFSFSNYRMHWLRQASGKGLEWVGVITVKSDNYGANYAASVKGRFTISRDDSKSSVYLQMNSLKTEDTAVYYCTSLGRVAYWGQGTLVTVSS

SEQ ID NO: 71 - protein, 6 aa, synthetic construct
SGYYWN

SEQ ID NO: 72 - protein, 16 aa, synthetic construct
YISYDGSNNYNPSLKN

SEQ ID NO: 73 - protein, 11 aa, synthetic construct
QIGLRRGAMDY

SEQ ID NO: 74 - protein, 11 aa, synthetic construct
KASDHINTWLA

SEQ ID NO: 75 - protein, 7 aa, synthetic construct
GSTSLKA

SEQ ID NO: 76 - protein, 9 aa, synthetic construct
QQYWGIPFT

SEQ ID NO: 77 - protein, 16 aa, synthetic construct
YTSYDGSNNYNPSFKN

SEQ ID NO: 78 - protein, 11 aa, synthetic construct
QIGLRRGAMDH

SEQ ID NO: 79 - protein, 11 aa, synthetic construct
KASDHINNWLA

SEQ ID NO: 80 - protein, 7 aa, synthetic construct
GSTSLRA

SEQ ID NO: 81 - protein, 11 aa, synthetic construct
QVGLRRGAMDY

SEQ ID NO: 82 - protein, 9 aa, synthetic construct
QQYWSIPYT

SEQ ID NO: 83 - protein, 5 aa, synthetic construct
NYRMH

SEQ ID NO: 84 - protein, 19 aa, synthetic construct
VITVKSDNYGANYAESVKG

SEQ ID NO: 85 - protein, 6 aa, synthetic construct
LGRVAY

SEQ ID NO: 86 - protein, 11 aa, synthetic construct
KASQNVDTNVV

SEQ ID NO: 87 - protein, 7 aa, synthetic construct
SASSRYS

SEQ ID NO: 88 - protein, 9 aa, synthetic construct
QQHNSFPLT

SEQ ID NO: 89 - protein, 19 aa, synthetic construct
RIRSKSNFYATYYADSVKD

SEQ ID NO: 90 - protein, 16 aa, synthetic construct
RSSKSLLHSNGNTYLY

SEQ ID NO: 91 - protein, 19 aa, synthetic construct
RIRSKSNYYATYYADSVKD

SEQ ID NO: 92 - protein, 16 aa, synthetic construct
YISYDGTNNYNPSLKN

SEQ ID NO: 93 - protein, 11 aa, synthetic construct
QVGLRRAWFAY

SEQ ID NO: 94 - protein, 11 aa, synthetic construct
KASDHINYWLA

SEQ ID NO: 95 - protein, 7 aa, synthetic construct
GATSLET

SEQ ID NO: 96 - protein, 8 aa, synthetic construct
HQYWSTPT

SEQ ID NO: 97 - protein, 19 aa, synthetic construct
RIRSKSNNYATYYADSVKD

SEQ ID NO: 98 - protein, 12 aa, synthetic construct
HGRSGYLYAMDY

SEQ ID NO: 99 - protein, 16 aa, synthetic construct
RSSKSLLHSNGITYLY

SEQ ID NO: 100 - protein, 7 aa, synthetic construct
QMSNLAS

SEQ ID NO: 101 - protein, 9 aa, synthetic construct
AQSLELPLT

SEQ ID NO: 102 - protein, 16 aa, synthetic construct
RSSKSLLHSQGNTYLY

SEQ ID NO: 103 - protein, 16 aa, synthetic construct
RSSKSLLHSIGNTYLY

SEQ ID NO: 104 - protein, 16 aa, synthetic construct
RSSKSLLHSSGNTYLY

SEQ ID NO: 105 - protein, 16 aa, synthetic construct
RSSKSLLHSNINTYLY

SEQ ID NO: 106 - protein, 16 aa, synthetic construct
RSSKSLLHSNVNTYLY

SEQ ID NO: 107 - protein, 16 aa, synthetic construct
RSSKSLLHSSGITYLY

SEQ ID NO: 108 - protein, 16 aa, synthetic construct
RSSKSLLHSYGITYLY

SEQ ID NO: 109 - protein, 16 aa, synthetic construct
RSSKSLLHSNAITYLY

SEQ ID NO: 110 - protein, 7 aa, synthetic construct
QASNLAS

SEQ ID NO: 111 - protein, 7 aa, synthetic construct
QTSNLAS

SEQ ID NO: 112 - protein, 12 aa, synthetic construct
HGRSGYLYAADY

SEQ ID NO: 113 - protein, 12 aa, synthetic construct
HGRSGYLYAFDY

SEQ ID NO: 114 - protein, 19 aa, synthetic construct
RIRSKSNFYATYYAASVKG

SEQ ID NO: 115 - protein, 5 aa, synthetic construct
TYAMH

SEQ ID NO: 116 - protein, 19 aa, synthetic construct
VITVKSDNYGANYAASVKG

SEQ ID NO: 117 - protein, 5 aa, synthetic construct
NYAMH

SEQ ID NO: 118 - protein, 19 aa, synthetic construct
RITVKSDNYGTAYAASVKG

SEQ ID NO: 119 - protein, 330 aa, synthetic construct [sequence omitted]

SEQ ID NO: 120 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK